SEQUENCE LISTING (107 sequences)

SEQ ID NO: 1 - DNA, 508 bp, Artificial Sequence
  cytomegalovirus (CMV) promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 2 - DNA, 60 bp, Artificial Sequence
  truncated form of the CMV promoter
aggtaggcgtgtacggtgggaggtctatataagcagagctggtttagtgaaccgtcagat

SEQ ID NO: 3 - DNA, 583 bp, Artificial Sequence
  fusion of the CMV early enhancer element and the first intron of the chicken beta-actin gene (CAG)
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - DNA, 2039 bp, Artificial Sequence
  vitelliform macular dystrophy protein-2 (VMD2) promoter
aattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggcacagccagccagtgtggctgcagcaaaacacacagggaaagaatggagggggcatcaatcactgcttcagtaaatttttattgagcgccttctacgagaacacaagaggagcttccattctgaggaggaaacaggcaggaaacaggcagatatcctgtataatttcaagtagtgataagtgctctctagaaatatcaagcaaggtgaggagacacagagcaccggtggcagtggggctctatttccaggttggatggttgggaacatcctttctaaagggaacctggagtgggaaggaaccatgcaggtatctcaggaagagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggagctaacgaacaagatgggctgagaacactgcctagcccagaggacctgagcttagtgtgtagacattgctgctgttactgcctttgtcattgtattatttatttatttatttatttatttttagacagagttttgctcttcttacccaggctggagtgcaatggcgtgatctcagctcactgcaacctccacctcctgggttcaagcgattctcctgcctcagcctcctgagtagctgggattacaggcacccgcaccacgcctggataatttttttgtatttttagtagagacagggtttcaccatgttggccaggctggtctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggccttggagaccacttcatccacctcctagggtccctatgggagagttgaggtccagagcagggaagggtcctgacaggctctgaccagggcctctgatccctacaaacccccaatcggtgtccctctctaccaggacccaagcccacctgctgcagcccactgcctggcc

SEQ ID NO: 5 - DNA, 623 bp, Artificial Sequence
  truncated form of the VMD2 promoter
aattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 6 - DNA, 462 bp, Artificial Sequence
  truncated form of the VMD2 promoter
tcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 7 - DNA, 500 bp, Artificial Sequence
  human phosphoglycerate kinase-1 (PGK) promoter
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 8 - DNA, 1182 bp, Artificial Sequence
  EF1alpha promoter
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - DNA, 230 bp, Artificial Sequence
  EF1alpha without the large intron promoter
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 10 - protein, 252 aa, Artificial Sequence
  an anti-VEGF single chain variable fragment (SCVF-1)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRGGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 11 - DNA, 756 bp, Artificial Sequence
  nucleic acid sequence encoding SCVF-1
gatatccagctgacccagagcccttctagcctgtccgcctctgtgggcgacagggtgaccatcacatgtagcgcctcccaggatatctccaactatctgaattggtaccagcagaagccaggcaaggcccccaaggtgctgatctatttcacctcctctctgcacagcggcgtgccatccagattctctggcagcggctccggcaccgactttaccctgacaatcagctccctgcagccagaggatttcgccacatactattgccagcagtacagcaccgtgccctggacatttggccagggcaccaaggtggagatcaagaggggaggaggaggaggatctggaggaggaggcagcggcggcggcggctccggcggcggcggctctgaggtgcagctggtggagtccggaggaggcctggtgcagccaggaggatctctgaggctgagctgtgccgcctccggctatgacttcacccactacggaatgaactgggtgcgccaggcacctggcaagggactggagtgggtgggctggatcaatacctatacaggcgagccaacctacgccgccgactttaagcggcggttcaccttcagcctggatacctctaagagcacagcctacctgcagatgaactccctgagggcagaggacaccgccgtgtactattgcgccaagtatccttactattacggcacaagccactggtacttcgacgtgtggggacagggcaccctggtgacagtgtctagc

SEQ ID NO: 12 - protein, 252 aa, Artificial Sequence
  anti-VEGF single chain variable fragment (SCVF-2)
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRGGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 13 - DNA, 756 bp, Artificial Sequence
  nucleic acid sequence encoding SCVF-2
gacatccagatgacccagagccccagcagcctgagcgccagcgtgggcgaccgcgtgaccatcacctgcagcgccagccaggacatcagcaactacctgaactggtaccagcagaagcccggcaaggcccccaaggtgctgatctacttcaccagcagcctgcacagcggcgtgcccagccgcttcagcggcagcggcagcggcaccgacttcaccctgaccatcagcagcctgcagcccgaggacttcgccacctactactgccagcagtacagcaccgtgccctggaccttcggccagggcaccaaggtggagatcaagcgcggcggcggcggcggcagcggcggcggcggcagcggcggcggcggcagcggcggcggcggcagcgaggtgcagctggtggagagcggcggcggcctggtgcagcccggcggcagcctgcgcctgagctgcgccgccagcggctacaccttcaccaactacggcatgaactgggtgcgccaggcccccggcaagggcctggagtgggtgggctggatcaacacctacaccggcgagcccacctacgccgccgacttcaagcgccgcttcaccttcagcctggacaccagcaagagcaccgcctacctgcagatgaacagcctgcgcgccgaggacaccgccgtgtactactgcgccaagtacccccactactacggcagcagccactggtacttcgacgtgtggggccagggcaccctggtgaccgtgagcagc

SEQ ID NO: 14 - protein, 255 aa, Artificial Sequence
  anti-VEGF single chain variable fragment (SCFV-3)
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 15 - DNA, 765 bp, Artificial Sequence
  nucleic acid sequence encoding SCFV-3
atggagatcgtgatgacccagagccccagcaccctgagcgccagcgtgggcgaccgcgtgatcatcacctgccaggccagcgagatcatccacagctggctggcctggtaccagcagaagcccggcaaggcccccaagctgctgatctacctggccagcaccctggccagcggcgtgcccagccgcttcagcggcagcggcagcggcgccgagttcaccctgaccatcagcagcctgcagcccgacgacttcgccacctactactgccagaacgtgtacctggccagcaccaacggcgccaacttcggccagggcaccaagctgaccgtgctgggcggcggcggcggcagcggcggcggcggcagcggcggcggcggcagcggcggcggcggcagcgaggtgcagctggtggagagcggcggcggcctggtgcagcccggcggcagcctgcgcctgagctgcgccgccagcggctacaccttcaccaactacggcatgaactgggtgcgccaggcccccggcaagggcctggagtgggtgggctggatcaacacctacaccggcgagcccacctacgccgccgacttcaagcgccgcttcaccttcagcctggacaccagcaagagcaccgcctacctgcagatgaacagcctgcgcgccgaggacaccgccgtgtactactgcgccaagtacccccactactacggcagcagccactggtacttcgacgtgtggggccagggcaccctggtgaccgtgagcagc

SEQ ID NO: 16 - protein, 249 aa, Artificial Sequence
  anti-VEGF single chain variable fragment (SCVF-4)
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 17 - DNA, 747 bp, Artificial Sequence
  nucleic acid sequence encoding SCVF-4
gacatccagatgacccagagccccagcagcctgagcgccagcgtgggcgaccgcgtgaccatcacctgcagcgccagccaggacatcagcaactacctgaactggtaccagcagaagcccggcaaggcccccaaggtgctgatctacttcaccagcagcctgcacagcggcgtgcccagccgcttcagcggcagcggcagcggcaccgacttcaccctgaccatcagcagcctgcagcccgaggacttcgccacctactactgccagcagtacagcaccgtgccctggaccttcggccagggcaccaaggtggagatcaagcgcggcggcggcggcggcagcggcggcggcggcagcggcggcggcggcagcggcggcggcggcagcgaggtgcagctggtggagagcggcggcggcctggtgcagcccggcggcagcctgcgcctgagctgcaccgccagcggcttcagcctgaccgactactactacatgacctgggtgcgccaggcccccggcaagggcctggagtgggtgggcttcatcgaccccgacgacgacccctactacgccacctgggccaagggccgcttcaccatcagccgcgacaacagcaagaacaccctgtacctgcagatgaacagcctgcgcgccgaggacaccgccgtgtactactgcgccggcggcgaccacaacagcggctggggcctggacatctggggccagggcaccctggtgaccgtgagcagc

SEQ ID NO: 18 - protein, 431 aa, Artificial Sequence
  VEGF capture protein-1
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - DNA, 1293 bp, Artificial Sequence
  nucleic acid sequence encoding VEGF capture protein-1
agcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggc

SEQ ID NO: 20 - protein, 431 aa, Artificial Sequence
  VEGF capture protein-2
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 21 - DNA, 1293 bp, Artificial Sequence
  nucleic acid sequence encoding VEGF capture protein-2
agcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggc

SEQ ID NO: 22 - protein, 242 aa, Artificial Sequence
  anti-C3b single chain variable fragment
DIQMTQSPSSLSASVGDRVTITCRASQDVSTAVAWYQQKPGKAPKLLIYSASFLYSGVPSRFSGSGSGTDFTLTISSLQPEDFATYYCQQSYATLPTFEQGTKVEIKRGGGGGSGGGGSGGGGSGGGGSEVQLVESGGGLVQPGGSLRLSCAASGFSFTSSSVSPGKGLEWVGLIYPYNGFNYYADSVKGRFTISADTSLQMNSLRAEDTAVYYCARNALYGSGGYYAMDYWGQGTLVTVSS

SEQ ID NO: 23 - DNA, 726 bp, Artificial Sequence
  nucleic acid sequence encoding anti-C3b single chain variable fragment
gacatccagatgacccagagccccagcagcctgagcgccagcgtgggcgaccgcgtgaccatcacctgccgcgccagccaggacgtaagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagagctacgccaccctgcccaccttcgagcagggcaccaaggtggagatcaagcgcggcggcggcggcggcagcggcggcggcggcagcggcggcggcggcagcggcggcggcggcagcgaggtgcagctggtggagagcggcggcggcctggtgcagcccggcggcagcctgcgcctgagctgcgccgccagcggcttcagcttcaccagcagcagcgtgagccccggcaagggcctggagtgggtgggcctgatctacccctacaacggcttcaactactacgccgacagcgtgaagggccgcttcaccatcagcgccgacaccagcctgcagatgaacagcctgcgcgccgaggacaccgccgtgtactactgcgcccgcaacgccctgtacggcagcggcggctactacgccatggactactggggccagggcaccctggtgaccgtgagcagc

SEQ ID NO: 24 - protein, 250 aa, Artificial Sequence
  anti-Bb single chain variable fragment
DVQITQSPSYLAASPGETITINCRASKSISKYLAWYQDKPGKTNKLLIYSGSTLQSGIPSRFSGSGSGTDFTLTISSLEPEDFAMYYCQQHDEYPWTFGGGTKLEIKRGGGGGSGGGGSGGGGSGGGGSQVQLQQSGAELAKPGASVRMSCKASGYTFTNYWIHWVKQRPGQGLEWIGYINPNTGYNDYNQKFKDKATLTADKSSSTVYMQLSSLTSEDSAVYYCARGGQLGLRRAMDYWGQGTSVTVSS

SEQ ID NO: 25 - DNA, 750 bp, Artificial Sequence
  nucleic acid sequence encoding anti-Bb single chain variable fragment
gacgtgcagatcacccagagccccagctacctggccgccagccccggcgagaccatcaccatcaactgccgcgccagcaagagcatcagcaagtacctggcctggtaccaggacaagcccggcaagaccaacaagctgctgatctacagcggcagcaccctgcagagcggcatccccagccgcttcagcggcagcggcagcggcaccgacttcaccctgaccatcagcagcctggagcccgaggacttcgccatgtactactgccagcagcacgacgagtacccctggaccttcggcggcggcaccaagctggagatcaagcgcggcggcggcggcggcagcggcggcggcggcagcggcggcggcggcagcggcggcggcggcagccaggtgcagctgcagcagagcggcgccgagctggccaagcccggcgccagcgtgcgcatgagctgcaaggccagcggctacaccttcaccaactactggatccactgggtgaagcagcgccccggccagggcctggagtggatcggctacatcaaccccaacaccggctacaacgactacaaccagaagttcaaggacaaggccaccctgaccgccgacaagagcagcagcaccgtgtacatgcagctgagcagcctgaccagcgaggacagcgccgtgtactactgcgcccgcggcggccagctgggcctgcgccgcgccatggactactggggccagggcaccagcgtgaccgtgagcagc

SEQ ID NO: 26 - protein, 320 aa, Artificial Sequence
  soluble (non-membrane bound) form of CD55 (sCD55)
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSG

SEQ ID NO: 27 - DNA, 960 bp, Artificial Sequence
  nucleic acid sequence encoding sCD55
gactgcggcctgccccccgacgtgcccaacgcccagcccgccctggagggccgcaccagcttccccgaggacaccgtgatcacctacaagtgcgaggagagcttcgtgaagatccccggcgagaaggacagcgtgatctgcctgaagggcagccagtggagcgacatcgaggagttctgcaaccgcagctgcgaggtgcccacccgcctgaacagcgccagcctgaagcagccctacatcacccagaactacttccccgtgggcaccgtggtggagtacgagtgccgccccggctaccgccgcgagcccagcctgagccccaagctgacctgcctgcagaacctgaagtggagcaccgccgtggagttctgcaagaagaagagctgccccaaccccggcgagatccgcaacggccagatcgacgtgcccggcggcatcctgttcggcgccaccatcagcttcagctgcaacaccggctacaagctgttcggcagcaccagcagcttctgcctgatcagcggcagcagcgtgcagtggagcgaccccctgcccgagtgccgcgagatctactgccccgcccccccccagatcgacaacggcatcatccagggcgagcgcgaccactacggctaccgccagagcgtgacctacgcctgcaacaagggcttcaccatgatcggcgagcacagcatctactgcaccgtgaacaacgacgagggcgagtggagcggccccccccccgagtgccgaggcaagagcctgaccagcaaggtgccccccaccgtgcagaagcccaccaccgtgaacgtgcccaccaccgaggtgagccccaccagccagaagaccaccaccaagaccaccacccccaacgcccaggccacccgcagcacccccgtgagccgcaccaccaagcacttccacgagaccacccccaacaagggcagcggcaccaccagcggc

SEQ ID NO: 28 - protein, 312 aa, Artificial Sequence
  human complement factor H related protein-1 (CFHR1)
EATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 29 - DNA, 936 bp, Artificial Sequence
  nucleic acid sequence encoding CFHR1
gaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgctcatatactgtcgagacagatgagtaaatatccatctggtgagagagtacgttatgaatgtaggagcccttatgaaatgtttggggatgaagaagtgatgtgtttaaatggaaactggacagaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaaga

SEQ ID NO: 30 - DNA, 936 bp, Artificial Sequence
  codon optimised nucleic acid sequence encoding CFHR1
gaggccaccttctgcgacttccccaagatcaaccacggcatcctgtacgacgaggagaagtacaagcccttcagccaggtgcccaccggcgaggtgttctactacagctgcgagtacaacttcgtgagccccagcaagagcttctggacccgcatcacctgcaccgaggagggctggagccccacccccaagtgcctgcgcctgtgcttcttccccttcgtggagaacggccacagcgagagcagcggccagacccacctggagggcgacaccgtgcagatcatctgcaacaccggctaccgcctgcagaacaacgagaacaacatcagctgcgtggagcgcggctggagcaccccccccaagtgccgcagcaccgacaccagctgcgtgaacccccccaccgtgcagaacgcccacatcctgagccgccagatgagcaagtaccccagcggcgagcgcgtgcgctacgagtgccgcagcccctacgagatgttcggcgacgaggaggtgatgtgcctgaacggcaactggaccgagcccccccagtgcaaggacagcaccggcaagtgcggcccccccccccccatcgacaacggcgacatcaccagcttccccctgagcgtgtacgcccccgccagcagcgtggagtaccagtgccagaacctgtaccagctggagggcaacaagcgcatcacctgccgcaacggccagtggagcgagccccccaagtgcctgcacccctgcgtgatcagccgcgagatcatggagaactacaacatcgccctgcgctggaccgccaagcagaagctgtacctgcgcaccggcgagagcgccgagttcgtgtgcaagcgcggctaccgcctgagcagccgcagccacaccctgcgcaccacctgctgggacggcaagctggagtaccccacctgcgccaagcgc

SEQ ID NO: 31 - protein, 310 aa, Artificial Sequence
  soluble (non-membrane-bound) form of CD46 (sCD46)
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDV

SEQ ID NO: 32 - DNA, 930 bp, Artificial Sequence
  nucleic acid sequence encoding sCD46
tgcgaggagccccccaccttcgaggccatggagctgatcggcaagcccaagccctactacgagatcggcgagcgcgtggactacaagtgcaagaagggctacttctacatcccccccctggccacccacaccatctgcgaccgcaaccacacctggctgcccgtgagcgacgacgcctgctaccgcgagacctgcccctacatccgcgaccccctgaacggccaggccgtgcccgccaacggcacctacgagttcggctaccagatgcacttcatctgcaacgagggctactacctgatcggcgaggagatcctgtactgcgagctgaagggcagcgtggccatctggagcggcaagccccccatctgcgagaaggtgctgtgcacccccccccccaagatcaagaacggcaagcacaccttcagcgaggtggaggtgttcgagtacctggacgccgtgacctacagctgcgaccccgcccccggccccgaccccttcagcctgatcggcgagagcaccatctactgcggcgacaacagcgtgtggagccgcgccgcccccgagtgcaaggtggtgaagtgccgcttccccgtggtggagaacggcaagcagatcagcggcttcggcaagaagttctactacaaggccaccgtgatgttcgagtgcgacaagggcttctacctggacggcagcgacaccatcgtgtgcgacagcaacagcacctgggacccccccgtgcccaagtgcctgaaggtgctgccccccagcagcaccaagccccccgccctgagccacagcgtgagcaccagcagcaccaccaagagccccgccagcagcgccagcggcccccgccccacctacaagccccccgtgagcaactaccccggctaccccaagcccgaggagggcatcctggacagcctggacgtg

SEQ ID NO: 33 - protein, 249 aa, Artificial Sequence
  anti-CTGF single chain variable fragment (anti-CTGF SCVF-1)
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRGGGGGSGGGGSGGGGSGGGGSEGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSS

SEQ ID NO: 34 - DNA, 747 bp, Artificial Sequence
  nucleic acid sequence encoding anti-CTGF SCVF-1
gacatccagatgacccagagccccagcagcctgagcgccagcgtgggcgaccgcgtgaccatcacctgccgcgccagccagggcatcagcagctggctggcctggtaccagcagaagcccgagaaggcccccaagagcctgatctacgccgccagcagcctccagagcggcgtgcccagccgcttcagcggcagcggcagcggcaccgacttcaccctgaccatcagcagcctccagcccgaggacttcgccacctactactgccagcagtacaacagctacccccccaccttcggccagggcaccaagctggagatcaagcgcggcggcggcggcggcagcggcggcggcggcagcggcggcggcggcagcggcggcggcggcagcgagggccagctggtgcagagcggcggcggcctggtgcaccccggcggcagcctgcgcctgagctgcgccggcagcggcttcaccttcagcagctacggcatgcactgggtgcgccaggcccccggcaagggcctggagtgggtgagcggcatcggcaccggcggcggcacctacagcaccgacagcgtgaagggccgcttcaccatcagccgcgacaacgccaagaacagcctgtacctccagatgaacagcctgcgcgccgaggacatggccgtgtactactgcgcccgcggcgactactacggcagcggcagcttcttcgactgctggggccagggcaccctggtgaccgtgagcagc

SEQ ID NO: 35 - protein, 241 aa, Artificial Sequence
  anti-CTGF single chain variable fragment (anti-CTGF SCVF-2)
AEVQLVESGGGVVRPGGSLRLSCAASGFTFDDYGMSWVRQAPGKGLEWVSYISSSGSTIYYADSVKGRFTISRDNAKNSLYLQMNSLRAEDTAVYYCARGITETWGQGTLVTVSSGGGGSGGGGSGGSALQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLGAVFGGGTKLTVLGA

SEQ ID NO: 36 - DNA, 723 bp, Artificial Sequence
  nucleic acid sequence encoding the anti-CTGF SCVF-2
gccgaggtgcagctggtggagagcggcggcggcgtggtgcggcctggcggcagcctgcgcctgagctgcgccgccagcggcttcaccttcgacgactacggcatgagctgggtgcgccaggctccaggcaagggcctggagtgggtgagctacatcagcagcagcggcagcaccatctactacgccgacagcgtgaagggccgcttcaccatcagccgcgacaacgccaagaacagcctgtacctgcagatgaacagcctgcgcgccgaggacaccgccgtgtactactgcgcacgaggcatcaccgagacctggggccagggcaccctggtgaccgtgagcagcggcggcggcggcagcggcggcggcggcagcggcggcagcgccctgcagagcgtgctgacccagccacccagcgccagcggcacccctggccagcgcgtgaccatcagctgcagcggcagcagcagcaacatcggcagcaacaccgtgaactggtaccagcagctgcccggcaccgcgcctaagctgctgatctacagcaacaaccagcgccccagcggcgtgcccgaccgcttcagcggcagcaagagcggcaccagcgccagcctggccatcagcggcctgcagagcgaggacgaggccgactactactgcgccgcctgggacgacagcctgggcgccgtgttcggcggcggcaccaagctgaccgtgctgggcgcc

SEQ ID NO: 37 - protein, 19 aa, Artificial Sequence
  P2A peptide spacer sequence
ATNFSLLKQAGDVEENPGP

SEQ ID NO: 38 - DNA, 57 bp, Artificial Sequence
  nucleic acid sequence encoding P2A peptide spacer sequence
gccaccaacttcagcctgctgaagcaggccggcgacgtggaggagaaccccggcccc

SEQ ID NO: 39 - protein, 20 aa, Artificial Sequence
  E2A peptide spacer sequence
QCTNYALLKLAGDVESNPGP

SEQ ID NO: 40 - DNA, 60 bp, Artificial Sequence
  nucleic acid sequence encoding E2A peptide spacer sequence
cagtgcaccaactacgccctgctgaagctggccggcgacgtggagagcaaccccggcccc

SEQ ID NO: 41 - protein, 18 aa, Artificial Sequence
  T2A peptide spacer sequence
EGRGSLLTCGDVEENPGP

SEQ ID NO: 42 - DNA, 54 bp, Artificial Sequence
  nucleic acid sequence encoding T2A peptide spacer sequence
gagggccgcggcagcctgctgacctgcggcgacgtggaggagaaccccggcccc

SEQ ID NO: 43 - protein, 22 aa, Artificial Sequence
  F2A peptide spacer sequence
VKQTLNFDLLKLAGDVESNPGP

SEQ ID NO: 44 - DNA, 66 bp, Artificial Sequence
  nucleic acid sequence encoding F2A peptide spacer sequence
gtgaagcagaccctgaacttcgacctgctgaagctggccggcgacgtggagagcaaccccggcccc

SEQ ID NO: 45 - protein, 6 aa, Artificial Sequence
  viral-2A removal sequence consensus sequence
  VARIANT (1)..(1): X = basic amino acid
  VARIANT (2)..(2): X = basic amino acid
  VARIANT (3)..(3): X = hydrophilic amino acid
  VARIANT (4)..(4): X = basic amino acid
  VARIANT (5)..(5): X = basic amino acid
XXXXXS

SEQ ID NO: 46 - protein, 9 aa, Artificial Sequence
  viral-2A removal sequence wherein the hydrophilic amino acid is serine (S)
RRSKRSGSG

SEQ ID NO: 47 - DNA, 27 bp, Artificial Sequence
  nucleic acid sequence encoding viral-2A removal sequence wherein the hydrophilic amino acid is serine (S)
cgccgcagcaagcgcagcggcagcggc

SEQ ID NO: 48 - protein, 9 aa, Artificial Sequence
  viral-2A removal sequence wherein the hydrophilic amino acid is threonine (T)
RRTKRSGSG

SEQ ID NO: 49 - DNA, 27 bp, Artificial Sequence
  nucleic acid sequence encoding viral-2A removal sequence wherein the hydrophilic amino acid is threonine (T)
cgccgcaccaagcgcagcggcagcggc

SEQ ID NO: 50 - protein, 7 aa, Artificial Sequence
  viral-2A removal sequence
RVRRGSG

SEQ ID NO: 51 - DNA, 21 bp, Artificial Sequence
  nucleic acid sequence encoding viral-2A removal sequence of SEQ ID NO 50
cgcgtgcgccgcggcagcggc

SEQ ID NO: 52 - protein, 8 aa, Artificial Sequence
  gelatinase MMP-2 recognition sequence
GPQGIAGQ

SEQ ID NO: 53 - protein, 8 aa, Artificial Sequence
  gelatinase MMP-2 recognition sequence
GPLGIAGA

SEQ ID NO: 54 - protein, 8 aa, Artificial Sequence
  gelatinase MMP-2 recognition sequence
GPQGLLGQ

SEQ ID NO: 55 - protein, 11 aa, Artificial Sequence
  gelatinase MMP-2 recognition sequence and the tripeptide GSG linker sequence
GPQGIAGQGSG

SEQ ID NO: 56 - DNA, 33 bp, Artificial Sequence
  nucleic acid sequence encoding gelatinase MMP-2 recognition sequence and the tripeptide GSG linker sequence
ggccccctgggcatcgccggccagggcagcggc

SEQ ID NO: 57 - protein, 8 aa, Artificial Sequence
  renin recognition sequence
HPFHLVYS

SEQ ID NO: 58 - protein, 9 aa, Artificial Sequence
  renin recognition sequence
HPFHLLVYS

SEQ ID NO: 59 - protein, 12 aa, Artificial Sequence
  renin recognition sequence and the tripeptide GSG linker sequence
HPFHLLVYSGSG

SEQ ID NO: 60 - DNA, 36 bp, Artificial Sequence
  nucleic acid sequence encoding renin recognition sequence and the tripeptide GSG linker sequence
cgccccttccacctgctggtcatccacggcagcggc

SEQ ID NO: 61 - DNA, 592 bp, Artificial Sequence
  Woodchuck Hepatitis Virus Post-transcriptional Regulatory Element (WPRE)
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 62 - DNA, 247 bp, Artificial Sequence
  truncated WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgt

SEQ ID NO: 63 - DNA, 224 bp, Artificial Sequence
  the simian virus-40 poly-A nucleotide sequence
agcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 64 - DNA, 169 bp, Artificial Sequence
  nucleotide sequence polyA component
ttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatcccccgatctg

SEQ ID NO: 65 - DNA, 128 bp, Artificial Sequence
  left ITR sequence taken from a commercially available recombinant AAV genome plasmid
cgcgctcgctcgctcactgaggccgcccgggcaaagcccgggcgtcgggcgacctttggtcgcccggcctcagtgagcgagcgagcgcgcagagagggagtggccaactccatcactaggggttcctt

SEQ ID NO: 66 - DNA, 128 bp, Artificial Sequence
  right ITR sequence taken from a commercially available recombinant AAV genome plasmid
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 67 - DNA, 121 bp, Artificial Sequence
  minute virus of mice (MVM) small intron
aggtacgatggcgcctccagctaaaagagctaaaagaggtaagggtttaagggatggttggttggtggggtattaatgtttaattacctgttttacaggcctgaaatcacttggttttagg

SEQ ID NO: 68 - DNA, 133 bp, Artificial Sequence
  sequence from the 5′-donor sites of the first intron of the human beta-globin gene and the branch and 3′-acceptor sites from the intron of an immunoglobulin gene heavy chain variable region
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 69 - DNA, 210 bp, Artificial Sequence
  fusion of 5′ and 3′ nucleotide components of the splice acceptor of the rabbit beta-globulin gene 1
gtgagcgggcgggacggcccttctcctccgggctgtaattagcgcttggtttaatgacggcttgtttcttttctgtggctgcgtgaaagccttgaggggctccgggaggctagagcctctgctaaccatgttcatgccttcttctttttcctacagctcctgggcaacgtgctggttattgtgctgtctcatcattttggcaaagaattc

SEQ ID NO: 70 - protein, 19 aa, Artificial Sequence
  signal peptide
MRRFLTVISFLLYFGCAFA

SEQ ID NO: 71 - DNA, 57 bp, Artificial Sequence
  nucleic acid sequence encoding signal peptide
atgcgccgcttcctgaccgtgatcagcttcctgctgtacttcggctgcgccttcgcc

SEQ ID NO: 72 - protein, 19 aa, Artificial Sequence
  signal peptide modified to enhance secretion from target cells
MRRLVLLLCIGALLGHSKA

SEQ ID NO: 73 - DNA, 57 bp, Artificial Sequence
  nucleic acid sequence encoding signal peptide modified to enhance secretion from target cells
atgcgccgcctggtgctgctgctgtgcatcggcgccctgctgggccacagcaaggcc

SEQ ID NO: 74 - protein, 12 aa, Artificial Sequence
  signal peptide
MRRLLTFISILA

SEQ ID NO: 75 - DNA, 45 bp, Artificial Sequence
  nucleic acid sequence encoding signal peptide of SEQ ID NO 74
atgaagaagctgctgatcctggccctggtgggcgccgccgtggcc

SEQ ID NO: 76 - protein, 18 aa, Artificial Sequence
  signal peptide
MRRLLTFISILALVGAFA

SEQ ID NO: 77 - DNA, 54 bp, Artificial Sequence
  nucleic acid sequence encoding signal peptide of SEQ ID NO 76
atgcgccgcctgctgaccttcatcagcatcctggccctggtgggcgccttcgcc

SEQ ID NO: 78 - protein, 19 aa, Artificial Sequence
  signal peptide
MRRLLTFISILALVGAAFA

SEQ ID NO: 79 - DNA, 57 bp, Artificial Sequence
  nucleic acid sequence encoding signal peptide of SEQ ID NO 78
atgcgccgcctgctgaccttcatcagcatcctggccctggtgggcgccgccttcgcc

SEQ ID NO: 80 - protein, 746 aa, Artificial Sequence
  VEGF capture protein-2-furin-P2A-anti-CTGF SCVF-1
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRFLTVISFLLYFGCAFADIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRGGGGGSGGGGSGGGGSGGGGSEGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSS

SEQ ID NO: 81 - DNA, 2241 bp, Artificial Sequence
  nucleic acid sequence encoding VEGF capture protein-2-furin-P2A-anti-CTGF SCVF-1
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgccgacatccagatgacccagagccccagcagcctgagcgccagcgtgggcgaccgcgtgaccatcacctgccgcgccagccagggcatcagcagctggctggcctggtaccagcagaagcccgagaaggcccccaagagcctgatctacgccgccagcagcctccagagcggcgtgcccagccgcttcagcggcagcggcagcggcaccgacttcaccctgaccatcagcagcctccagcccgaggacttcgccacctactactgccagcagtacaacagctacccccccaccttcggccagggcaccaagctggagatcaagcgcggcggcggcggcggcagcggcggcggcggcagcggcggcggcggcagcggcggcggcggcagcgagggccagctggtgcagagcggcggcggcctggtgcaccccggcggcagcctgcgcctgagctgcgccggcagcggcttcaccttcagcagctacggcatgcactgggtgcgccaggcccccggcaagggcctggagtgggtgagcggcatcggcaccggcggcggcacctacagcaccgacagcgtgaagggccgcttcaccatcagccgcgacaacgccaagaacagcctgtacctccagatgaacagcctgcgcgccgaggacatggccgtgtactactgcgcccgcggcgactactacggcagcggcagcttcttcgactgctggggccagggcaccctggtgaccgtgagcagctaa

SEQ ID NO: 82 - protein, 738 aa, Artificial Sequence
  VEGF capture protein-2-furin-P2A-anti-CTGF SCVF-2
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRFLTVISFLLYFGCAFAAEVQLVESGGGVVRPGGSLRLSCAASGFTFDDYGMSWVRQAPGKGLEWVSYISSSGSTIYYADSVKGRFTISRDNAKNSLYLQMNSLRAEDTAVYYCARGITETWGQGTLVTVSSGGGGSGGGGSGGSALQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLGAVFGGGTKLTVLGA

SEQ ID NO: 83 - DNA, 2217 bp, Artificial Sequence
  nucleic acid sequence encoding VEGF capture protein-2-furin-P2A-anti-CTGF SCVF-2
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgccgccgaggtgcagctggtggagagcggcggcggcgtggtgcggcctggcggcagcctgcgcctgagctgcgccgccagcggcttcaccttcgacgactacggcatgagctgggtgcgccaggctccaggcaagggcctggagtgggtgagctacatcagcagcagcggcagcaccatctactacgccgacagcgtgaagggccgcttcaccatcagccgcgacaacgccaagaacagcctgtacctgcagatgaacagcctgcgcgccgaggacaccgccgtgtactactgcgcacgaggcatcaccgagacctggggccagggcaccctggtgaccgtgagcagcggcggcggcggcagcggcggcggcggcagcggcggcagcgccctgcagagcgtgctgacccagccacccagcgccagcggcacccctggccagcgcgtgaccatcagctgcagcggcagcagcagcaacatcggcagcaacaccgtgaactggtaccagcagctgcccggcaccgcgcctaagctgctgatctacagcaacaaccagcgccccagcggcgtgcccgaccgcttcagcggcagcaagagcggcaccagcgccagcctggccatcagcggcctgcagagcgaggacgaggccgactactactgcgccgcctgggacgacagcctgggcgccgtgttcggcggcggcaccaagctgaccgtgctgggcgcctaa

SEQ ID NO: 84 - protein, 739 aa, Artificial Sequence
  VEGF capture protein 2-furin-P2A-anti-C3b SCVF
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRFLTVISFLLYFGCAFADIQMTQSPSSLSASVGDRVTITCRASQDVSTAVAWYQQKPGKAPKLLIYSASFLYSGVPSRFSGSGSGTDFTLTISSLQPEDFATYYCQQSYATLPTFEQGTKVEIKRGGGGGSGGGGSGGGGSGGGGSEVQLVESGGGLVQPGGSLRLSCAASGFSFTSSSVSPGKGLEWVGLIYPYNGFNYYADSVKGRFTISADTSLQMNSLRAEDTAVYYCARNALYGSGGYYAMDYWGQGTLVTVSS

SEQ ID NO: 85 - DNA, 2220 bp, Artificial Sequence
  nucleic acid sequence encoding VEGF capture protein 2-furin-P2A-anti-C3b SCVF
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgccgacatccagatgacccagagccccagcagcctgagcgccagcgtgggcgaccgcgtgaccatcacctgccgcgccagccaggacgtaagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagagctacgccaccctgcccaccttcgagcagggcaccaaggtggagatcaagcgcggcggcggcggcggcagcggcggcggcggcagcggcggcggcggcagcggcggcggcggcagcgaggtgcagctggtggagagcggcggcggcctggtgcagcccggcggcagcctgcgcctgagctgcgccgccagcggcttcagcttcaccagcagcagcgtgagccccggcaagggcctggagtgggtgggcctgatctacccctacaacggcttcaactactacgccgacagcgtgaagggccgcttcaccatcagcgccgacaccagcctgcagatgaacagcctgcgcgccgaggacaccgccgtgtactactgcgcccgcaacgccctgtacggcagcggcggctactacgccatggactactggggccagggcaccctggtgaccgtgagcagctga

SEQ ID NO: 86 - protein, 747 aa, Artificial Sequence
  VEGF capture protein 2-furin-P2A-anti-Bb SCVF
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRFLTVISFLLYFGCAFADVQITQSPSYLAASPGETITINCRASKSISKYLAWYQDKPGKTNKLLIYSGSTLQSGIPSRFSGSGSGTDFTLTISSLEPEDFAMYYCQQHDEYPWTFGGGTKLEIKRGGGGGSGGGGSGGGGSGGGGSQVQLQQSGAELAKPGASVRMSCKASGYTFTNYWIHWVKQRPGQGLEWIGYINPNTGYNDYNQKFKDKATLTADKSSSTVYMQLSSLTSEDSAVYYCARGGQLGLRRAMDYWGQGTSVTVSS

SEQ ID NO: 87 - DNA, 2244 bp, Artificial Sequence
  nucleic acid sequence encoding VEGF capture protein 2-furin-P2A-anti-Bb SCVF
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgccgacgtgcagatcacccagagccccagctacctggccgccagccccggcgagaccatcaccatcaactgccgcgccagcaagagcatcagcaagtacctggcctggtaccaggacaagcccggcaagaccaacaagctgctgatctacagcggcagcaccctgcagagcggcatccccagccgcttcagcggcagcggcagcggcaccgacttcaccctgaccatcagcagcctggagcccgaggacttcgccatgtactactgccagcagcacgacgagtacccctggaccttcggcggcggcaccaagctggagatcaagcgcggcggcggcggcggcagcggcggcggcggcagcggcggcggcggcagcggcggcggcggcagccaggtgcagctgcagcagagcggcgccgagctggccaagcccggcgccagcgtgcgcatgagctgcaaggccagcggctacaccttcaccaactactggatccactgggtgaagcagcgccccggccagggcctggagtggatcggctacatcaaccccaacaccggctacaacgactacaaccagaagttcaaggacaaggccaccctgaccgccgacaagagcagcagcaccgtgtacatgcagctgagcagcctgaccagcgaggacagcgccgtgtactactgcgcccgcggcggccagctgggcctgcgccgcgccatggactactggggccagggcaccagcgtgaccgtgagcagctga

SEQ ID NO: 88 - protein, 817 aa, Artificial Sequence
  VEGF capture protein 2-furin-P2A-sCD55
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRFLTVISFLLYFGCAFADCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSG

SEQ ID NO: 89 - DNA, 2454 bp, Artificial Sequence
  nucleic acid sequence encoding VEGF capture protein 2-furin-P2A-sCD55
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgccgactgcggcctgccccccgacgtgcccaacgcccagcccgccctggagggccgcaccagcttccccgaggacaccgtgatcacctacaagtgcgaggagagcttcgtgaagatccccggcgagaaggacagcgtgatctgcctgaagggcagccagtggagcgacatcgaggagttctgcaaccgcagctgcgaggtgcccacccgcctgaacagcgccagcctgaagcagccctacatcacccagaactacttccccgtgggcaccgtggtggagtacgagtgccgccccggctaccgccgcgagcccagcctgagccccaagctgacctgcctgcagaacctgaagtggagcaccgccgtggagttctgcaagaagaagagctgccccaaccccggcgagatccgcaacggccagatcgacgtgcccggcggcatcctgttcggcgccaccatcagcttcagctgcaacaccggctacaagctgttcggcagcaccagcagcttctgcctgatcagcggcagcagcgtgcagtggagcgaccccctgcccgagtgccgcgagatctactgccccgcccccccccagatcgacaacggcatcatccagggcgagcgcgaccactacggctaccgccagagcgtgacctacgcctgcaacaagggcttcaccatgatcggcgagcacagcatctactgcaccgtgaacaacgacgagggcgagtggagcggccccccccccgagtgccgaggcaagagcctgaccagcaaggtgccccccaccgtgcagaagcccaccaccgtgaacgtgcccaccaccgaggtgagccccaccagccagaagaccaccaccaagaccaccacccccaacgcccaggccacccgcagcacccccgtgagccgcaccaccaagcacttccacgagaccacccccaacaagggcagcggcaccaccagcggctaa

SEQ ID NO: 90 - protein, 817 aa, Artificial Sequence
  sCD55-furin-P2A- VEGF capture protein 2
MRRFLTVISFLLYFGCAFADCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGRRSKRSGSGATNFSLLKQAGDVEENPGPMRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 91 - DNA, 2454 bp, Artificial Sequence
  nucleic acid sequence encoding sCD55-furin-P2A- VEGF capture protein 2
atgcgccgcttcctgaccgtgatcagcttcctgctgtacttcggctgcgccttcgccgactgcggcctgccccccgacgtgcccaacgcccagcccgccctggagggccgcaccagcttccccgaggacaccgtgatcacctacaagtgcgaggagagcttcgtgaagatccccggcgagaaggacagcgtgatctgcctgaagggcagccagtggagcgacatcgaggagttctgcaaccgcagctgcgaggtgcccacccgcctgaacagcgccagcctgaagcagccctacatcacccagaactacttccccgtgggcaccgtggtggagtacgagtgccgccccggctaccgccgcgagcccagcctgagccccaagctgacctgcctgcagaacctgaagtggagcaccgccgtggagttctgcaagaagaagagctgccccaaccccggcgagatccgcaacggccagatcgacgtgcccggcggcatcctgttcggcgccaccatcagcttcagctgcaacaccggctacaagctgttcggcagcaccagcagcttctgcctgatcagcggcagcagcgtgcagtggagcgaccccctgcccgagtgccgcgagatctactgccccgcccccccccagatcgacaacggcatcatccagggcgagcgcgaccactacggctaccgccagagcgtgacctacgcctgcaacaagggcttcaccatgatcggcgagcacagcatctactgcaccgtgaacaacgacgagggcgagtggagcggccccccccccgagtgccgaggcaagagcctgaccagcaaggtgccccccaccgtgcagaagcccaccaccgtgaacgtgcccaccaccgaggtgagccccaccagccagaagaccaccaccaagaccaccacccccaacgcccaggccacccgcagcacccccgtgagccgcaccaccaagcacttccacgagaccacccccaacaagggcagcggcaccaccagcggccgccgcagcaagcgcagcggcagcggcgccaccaacttcagcctgctgaagcaggccggcgacgtggaggagaaccccggccccatgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggctaa

SEQ ID NO: 92 - protein, 809 aa, Artificial Sequence
  VEGF capture protein 2-furin-P2A-CFHR1
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRFLTVISFLLYFGCAFAEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 93 - DNA, 2430 bp, Artificial Sequence
  nucleic acid sequence encoding VEGF capture protein 2-furin-P2A-CFHR1
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgccgaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgctcatatactgtcgagacagatgagtaaatatccatctggtgagagagtacgttatgaatgtaggagcccttatgaaatgtttggggatgaagaagtgatgtgtttaaatggaaactggacagaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagataa

SEQ ID NO: 94 - protein, 809 aa, Artificial Sequence
  CFHR1-furin-P2A-VEGF capture protein 2
MRRFLTVISFLLYFGCAFAEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKRRRSKRSGSGATNFSLLKQAGDVEENPGPMRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 95 - DNA, 2430 bp, Artificial Sequence
  nucleic acid sequence encoding CFHR1-furin-P2A- VEGF capture protein 2
atgcgccgcttcctgaccgtgatcagcttcctgctgtacttcggctgcgccttcgccgaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgctcatatactgtcgagacagatgagtaaatatccatctggtgagagagtacgttatgaatgtaggagcccttatgaaatgtttggggatgaagaagtgatgtgtttaaatggaaactggacagaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagacgccgcagcaagcgcagcggcagcggcgccaccaacttcagcctgctgaagcaggccggcgacgtggaggagaaccccggccccatgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggctaa

SEQ ID NO: 96 - DNA, 4246 bp, Artificial Sequence
  recombinant vector (e.g. known as “IKC153P”)
cgcgctcgctcgctcactgaggccgcccgggcaaagcccgggcggcctcagtgagcgagcgagcgcgcagagagggagtggccaactccatcactaggggttccttgtagttaatgattaacccgccatgctacttatctacgtagccatgctctagacatggctcgacagatcgagctccaccgggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggaggctagagcctctgctaaccatgttcatgccttcttctttttcctacagctcctgggcaacgtgctggttattgtgctgtctcatcattttggcaaagaattccggactcgacccgctagcgccaccatgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgccgacatccagatgacccagagccccagcagcctgagcgccagcgtgggcgaccgcgtgaccatcacctgccgcgccagccagggcatcagcagctggctggcctggtaccagcagaagcccgagaaggcccccaagagcctgatctacgccgccagcagcctccagagcggcgtgcccagccgcttcagcggcagcggcagcggcaccgacttcaccctgaccatcagcagcctccagcccgaggacttcgccacctactactgccagcagtacaacagctacccccccaccttcggccagggcaccaagctggagatcaagcgcggcggcggcggcggcagcggcggcggcggcagcggcggcggcggcagcggcggcggcggcagcgagggccagctggtgcagagcggcggcggcctggtgcaccccggcggcagcctgcgcctgagctgcgccggcagcggcttcaccttcagcagctacggcatgcactgggtgcgccaggcccccggcaagggcctggagtgggtgagcggcatcggcaccggcggcggcacctacagcaccgacagcgtgaagggccgcttcaccatcagccgcgacaacgccaagaacagcctgtacctccagatgaacagcctgcgcgccgaggacatggccgtgtactactgcgcccgcggcgactactacggcagcggcagcttcttcgactgctggggccagggcaccctggtgaccgtgagcagctaagtatactactagtacgcggccgcaccggtgtacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtgaattcgagctaggtacagcttatcgataccgtcgacagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgctcgagggcatgcaacaacaacaattgcattcattttatgtttcaggttcagggggagatgtgggaggttttttaaagcaagtaaaacctctacaaatgtggtaaaatccgataaggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 97 - protein, 426 aa, Artificial Sequence
  CFHL1
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIR

SEQ ID NO: 98 - DNA, 1278 bp, Artificial Sequence
  nucleic acid sequence encoding CFHL1
gaggactgcaacgagctgcccccccgccgcaacaccgagatcctgaccggcagctggagcgaccagacctaccccgagggcacccaggccatctacaagtgccgccccggctaccgcagcctgggcaacgtgatcatggtgtgccgcaagggcgagtgggtggccctgaaccccctgcgcaagtgccagaagcgcccctgcggccaccccggcgacacccccttcggcaccttcaccctgaccggcggcaacgtgttcgagtacggcgtgaaggccgtgtacacctgcaacgagggctaccagctgctgggcgagatcaactaccgcgagtgcgacaccgacggctggaccaacgacatccccatctgcgaggtggtgaagtgcctgcccgtgaccgcccccgagaacggcaagatcgtgagcagcgccatggagcccgaccgcgagtaccacttcggccaggccgtgcgcttcgtgtgcaacagcggctacaagatcgagggcgacgaggagatgcactgcagcgacgacggcttctggagcaaggagaagcccaagtgcgtggagatcagctgcaagagccccgacgtgatcaacggcagccccatcagccagaagatcatctacaaggagaacgagcgcttccagtacaagtgcaacatgggctacgagtacagcgagcgcggcgacgccgtgtgcaccgagagcggctggcgccccctgcccagctgcgaggagaagagctgcgacaacccctacatccccaacggcgactacagccccctgcgcatcaagcaccgcaccggcgacgagatcacctaccagtgccgcaacggcttctaccccgccacccggggcaacaccgccaagtgcaccagcaccggctggatccccgccccccgctgcaccctgaagccctgcgactaccccgacatcaagcacggcggcctgtaccacgagaacatgcgccgcccctacttccccgtggccgtgggcaagtactacagctactactgcgacgagcacttcgagacccccagcggcagctactgggaccacatccactgcacccaggacggctggagccccgccgtgccctgcctgcgcaagtgctacttcccctacctggagaacggctacaaccagaactacggccgcaagttcgtgcagggcaagagcatcgacgtggcctgccaccccggctacgccctgcccaaggcccagaccaccgtgacctgcatggagaacggctggagccccaccccccgctgcatccgc

SEQ ID NO: 99 - DNA, 225 bp, Artificial Sequence
  bovine growth hormone poly-A nucleotide sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 100 - protein, 922 aa, Artificial Sequence
  VEGF capture protein 2-furin-P2A-CFHL1
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRLLTFISILALVGAFAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIR

SEQ ID NO: 101 - DNA, 2769 bp, Artificial Sequence
  nucleic acid sequence encoding VEGF capture protein 2-furin-P2A-CFHL1
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcctgctgaccttcatcagcatcctggccctggtgggcgccttcgccgaggactgcaacgagctgcccccccgccgcaacaccgagatcctgaccggcagctggagcgaccagacctaccccgagggcacccaggccatctacaagtgccgccccggctaccgcagcctgggcaacgtgatcatggtgtgccgcaagggcgagtgggtggccctgaaccccctgcgcaagtgccagaagcgcccctgcggccaccccggcgacacccccttcggcaccttcaccctgaccggcggcaacgtgttcgagtacggcgtgaaggccgtgtacacctgcaacgagggctaccagctgctgggcgagatcaactaccgcgagtgcgacaccgacggctggaccaacgacatccccatctgcgaggtggtgaagtgcctgcccgtgaccgcccccgagaacggcaagatcgtgagcagcgccatggagcccgaccgcgagtaccacttcggccaggccgtgcgcttcgtgtgcaacagcggctacaagatcgagggcgacgaggagatgcactgtagcgacgacggcttctggagcaaggagaagcccaagtgcgtggagatcagctgcaagagccccgacgtgatcaacggcagccccatcagccagaagatcatctacaaggagaacgagcgcttccagtacaagtgcaacatgggctacgagtacagcgagcgcggcgacgccgtgtgcaccgagagcggctggcgccccctgcccagctgcgaggagaagagctgcgacaacccctacatccccaacggcgactacagccccctgcgcatcaagcaccgcaccggcgacgagatcacctaccagtgccgcaacggcttctaccccgccacccgcggcaacaccgccaagtgcaccagcaccggctggatccccgccccccgctgcaccctgaagccctgcgactaccccgacatcaagcacggcggcctgtaccacgagaacatgcgccgcccctacttccccgtggccgtgggcaagtactacagctactactgcgacgagcacttcgagacccccagcggcagctactgggaccacatccactgcacccaggacggctggagccccgccgtgccctgcctgcgcaagtgctacttcccctacctggagaacggctacaaccagaactacggccgcaagttcgtgcagggcaagagcatcgacgtggcctgccaccccggctacgccctgcccaaggcccagaccaccgtgacctgcatggagaacggctggagccccaccccccgctgcatccgctag

SEQ ID NO: 102 - protein, 922 aa, Artificial Sequence
  CFHL1-furin-P2A-VEGF capture protein 2
MRRLLTFISILALVGAFAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRRRSKRSGSGATNFSLLKQAGDVEENPGPMRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 103 - DNA, 2766 bp, Artificial Sequence
  nucleic acid sequence encoding CFHL1-furin-P2A- VEGF capture protein 2
atgcgccgcctgctgaccttcatcagcatcctggccctggtgggcgccttcgccgaggactgcaacgagctgcccccccgccgcaacaccgagatcctgaccggcagctggagcgaccagacctaccccgagggcacccaggccatctacaagtgccgccccggctaccgcagcctgggcaacgtgatcatggtgtgccgcaagggcgagtgggtggccctgaaccccctgcgcaagtgccagaagcgcccctgcggccaccccggcgacacccccttcggcaccttcaccctgaccggcggcaacgtgttcgagtacggcgtgaaggccgtgtacacctgcaacgagggctaccagctgctgggcgagatcaactaccgcgagtgcgacaccgacggctggaccaacgacatccccatctgcgaggtggtgaagtgcctgcccgtgaccgcccccgagaacggcaagatcgtgagcagcgccatggagcccgaccgcgagtaccacttcggccaggccgtgcgcttcgtgtgcaacagcggctacaagatcgagggcgacgaggagatgcactgtagcgacgacggcttctggagcaaggagaagcccaagtgcgtggagatcagctgcaagagccccgacgtgatcaacggcagccccatcagccagaagatcatctacaaggagaacgagcgcttccagtacaagtgcaacatgggctacgagtacagcgagcgcggcgacgccgtgtgcaccgagagcggctggcgccccctgcccagctgcgaggagaagagctgcgacaacccctacatccccaacggcgactacagccccctgcgcatcaagcaccgcaccggcgacgagatcacctaccagtgccgcaacggcttctaccccgccacccgcggcaacaccgccaagtgcaccagcaccggctggatccccgccccccgctgcaccctgaagccctgcgactaccccgacatcaagcacggcggcctgtaccacgagaacatgcgccgcccctacttccccgtggccgtgggcaagtactacagctactactgcgacgagcacttcgagacccccagcggcagctactgggaccacatccactgcacccaggacggctggagccccgccgtgccctgcctgcgcaagtgctacttcccctacctggagaacggctacaaccagaactacggccgcaagttcgtgcagggcaagagcatcgacgtggcctgccaccccggctacgccctgcccaaggcccagaccaccgtgacctgcatggagaacggctggagccccaccccccgctgcatccgccgccgcagcaagcgcagcggcagcggcgccaccaacttcagcctgctgaagcaggccggcgacgtggaggagaaccccggccccatgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggc

SEQ ID NO: 104 - protein, 807 aa, Artificial Sequence
  VEGF capture protein 2-furin-P2A-sCD46
MRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGRRSKRSGSGATNFSLLKQAGDVEENPGPMRRFLTVISFLLYFGCAFACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDV

SEQ ID NO: 105 - DNA, 2424 bp, Artificial Sequence
  nucleic acid sequence encoding VEGF capture protein 2-furin-P2A-sCD46
atgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggccgccgcagcaagcgcagcggcagcggcgccaccaacttcagcctgctgaagcaggccggcgacgtggaggagaaccccggccccatgcgccgcttcctgaccgtgatcagcttcctgctgtacttcggctgcgccttcgcctgcgaggagccccccaccttcgaggccatggagctgatcggcaagcccaagccctactacgagatcggcgagcgcgtggactacaagtgcaagaagggctacttctacatcccccccctggccacccacaccatctgcgaccgcaaccacacctggctgcccgtgagcgacgacgcctgctaccgcgagacctgcccctacatccgcgaccccctgaacggccaggccgtgcccgccaacggcacctacgagttcggctaccagatgcacttcatctgcaacgagggctactacctgatcggcgaggagatcctgtactgcgagctgaagggcagcgtggccatctggagcggcaagccccccatctgcgagaaggtgctgtgcacccccccccccaagatcaagaacggcaagcacaccttcagcgaggtggaggtgttcgagtacctggacgccgtgacctacagctgcgaccccgcccccggccccgaccccttcagcctgatcggcgagagcaccatctactgcggcgacaacagcgtgtggagccgcgccgcccccgagtgcaaggtggtgaagtgccgcttccccgtggtggagaacggcaagcagatcagcggcttcggcaagaagttctactacaaggccaccgtgatgttcgagtgcgacaagggcttctacctggacggcagcgacaccatcgtgtgcgacagcaacagcacctgggacccccccgtgcccaagtgcctgaaggtgctgccccccagcagcaccaagccccccgccctgagccacagcgtgagcaccagcagcaccaccaagagccccgccagcagcgccagcggcccccgccccacctacaagccccccgtgagcaactaccccggctaccccaagcccgaggagggcatcctggacagcctggacgtgtaa

SEQ ID NO: 106 - protein, 807 aa, Artificial Sequence
  sCD46-furin-P2A-VEGF capture protein 2
MRRFLTVISFLLYFGCAFACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVRRSKRSGSGATNFSLLKQAGDVEENPGPMRRLLTFISILALVGAAFASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 107 - DNA, 2421 bp, Artificial Sequence
  nucleic acid sequence encoding sCD46-furin-P2A- VEGF capture protein 2
atgcgccgcttcctgaccgtgatcagcttcctgctgtacttcggctgcgccttcgcctgcgaggagccccccaccttcgaggccatggagctgatcggcaagcccaagccctactacgagatcggcgagcgcgtggactacaagtgcaagaagggctacttctacatcccccccctggccacccacaccatctgcgaccgcaaccacacctggctgcccgtgagcgacgacgcctgctaccgcgagacctgcccctacatccgcgaccccctgaacggccaggccgtgcccgccaacggcacctacgagttcggctaccagatgcacttcatctgcaacgagggctactacctgatcggcgaggagatcctgtactgcgagctgaagggcagcgtggccatctggagcggcaagccccccatctgcgagaaggtgctgtgcacccccccccccaagatcaagaacggcaagcacaccttcagcgaggtggaggtgttcgagtacctggacgccgtgacctacagctgcgaccccgcccccggccccgaccccttcagcctgatcggcgagagcaccatctactgcggcgacaacagcgtgtggagccgcgccgcccccgagtgcaaggtggtgaagtgccgcttccccgtggtggagaacggcaagcagatcagcggcttcggcaagaagttctactacaaggccaccgtgatgttcgagtgcgacaagggcttctacctggacggcagcgacaccatcgtgtgcgacagcaacagcacctgggacccccccgtgcccaagtgcctgaaggtgctgccccccagcagcaccaagccccccgccctgagccacagcgtgagcaccagcagcaccaccaagagccccgccagcagcgccagcggcccccgccccacctacaagccccccgtgagcaactaccccggctaccccaagcccgaggagggcatcctggacagcctggacgtgcgccgcagcaagcgcagcggcagcggcgccaccaacttcagcctgctgaagcaggccggcgacgtggaggagaaccccggccccatgcgccgcctgctgaccttcatcagcatcctggccctggtgggcgccgccttcgccagcgacaccggccgccccttcgtggagatgtacagcgagatccccgagatcatccacatgaccgagggccgcgagctggtgatcccctgccgcgtgaccagccccaacatcaccgtgaccctgaagaagttccccctggacaccctgatccccgacggcaagcgcatcatctgggacagccgcaagggcttcatcatcagcaacgccacctacaaggagatcggcctgctgacctgcgaggccaccgtgaacggccacctgtacaagaccaactacctgacccaccgccagaccaacaccatcatcgacgtggtgctgagccccagccacggcatcgagctgagcgtgggcgagaagctggtgctgaactgcaccgcccgcaccgagctgaacgtgggcatcgacttcaactgggagtaccccagcagcaagcaccagcacaagaagctggtgaaccgcgacctgaagacccagagcggcagcgagatgaagaagttcctgagcaccctgaccatcgacggcgtgacccgcagcgaccagggcctgtacacctgcgccgccagcagcggcctgatgaccaagaagaacagcaccttcgtgcgcgtgcacgagaaggacaagacccacacctgccccccctgccccgcccccgaggccgccggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctggcccaggactggctgaacggcaaggagtacaagtgcaaggtgagcaacaaggccctgggcgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaacgcctacacccagaagagcctgagcctgagccccggc

(19) United States
(12) Patent Application Publication
(10) Pub. No.: US 2024/0285800 A1
(43) Pub. Date: Aug. 29, 2024

(54) Retinal Disorders

(71) Applicant: Ikarovec Limited, Norwich (GB)

(72) Inventors: Peter Widdowson, Norwich (GB); Katie Binley, Norwich (GB)

(21) Appl. No.: 18/570,701

(22) PCT Filed: Jun. 17, 2022
(86) PCT No.: PCT/GB2022/051539

§ 371 Date: Dec. 15, 2023

(30) Foreign Application Priority Data
Jun. 18, 2021 (GB) 2108760.6
Mar. 25, 2022 (GB) 2204234.5

(51) Int. Cl. A61K 48/00; A61K 38/00; A61K 39/00; A61P 27/02; C07K 14/705; C07K 16/22; C12N 15/86

(52) U.S. Cl. CPC A61K 48/005; A61P 27/02; C07K 14/70596; C07K 16/22; C12N 15/86; A61K 38/00; A61K 2039/505; C07K 2317/622; C12N 2750/14143

(57) ABSTRACT

The invention relates to retinal disorders, and to genetic constructs and recombinant vectors comprising such constructs, and their use in gene therapy methods for treating, preventing or ameliorating a wide range of retinal disorders. The constructs and vectors are particularly useful for treating wet age-related macular degeneration (wet-AMD), i.e., neovascular age related macular degeneration. The invention extends to the use of the constructs and vectors for reducing vascular leakage and retinal cell damage. The invention also extends to pharmaceutical compositions per se, and their use in treating, preventing or ameliorating retinal disorders, and for reducing vascular leakage and retinal cell damage.

The present invention relates to retinal disorders, and to genetic constructs and recombinant vectors comprising such constructs, and their use in gene therapy methods for treating, preventing or ameliorating a wide range of retinal disorders. The constructs and vectors are particularly, although not exclusively, useful for treating wet age-related macular degeneration (wet-AMD), i.e., neovascular age related macular degeneration. The invention also extends to the use of the constructs and vectors for reducing vascular leakage and retinal cell damage. The invention also extends to pharmaceutical compositions per se, and their use in treating, preventing or ameliorating retinal disorders, and for reducing vascular leakage and retinal cell damage.

Macular degeneration, also known as age-related macular degeneration (AMD), is a common eye condition among people aged 50 and older, which affects the macula, a small part of the retina [1-3]. The macula is used for central and detailed vision, necessary for activities such as reading and driving. AMD is the leading cause of permanent, irreversible sight loss in adult populations across developed countries [4], affecting approximately 600,000 people in the U.K. [5], and estimated in 2013 to be the fourth most common cause of blindness after cataracts, preterm birth and glaucoma [6]. In 2015, AMD was estimated to affect 6.2 million people globally [7]. Approximately 0.4% of people between 50 and 60 have AMD, rising to 0.7% of people aged 60 to 70, 2.3% of those aged 70 to 80, and nearly 12% of people over 80 years old [1]. Accordingly, the incidence of AMD is also rising in line with a shift towards ageing populations [8]. AMD can be diagnosed as either ‘dry’ (non-neovascular) or ‘wet’ (exudative or neovascular) AMD [1,2]. Dry AMD, or geographic atrophy, is the more common form of AMD and accounts for 90% of cases. Dry AMD generally results in slower sight loss, whilst wet-AMD causes a relatively sudden change in vision, resulting in substantial visual loss [1,2].

A Canadian study concluded that moderate AMD caused a 40% decrease in quality of life, a decrease similar to that associated with permanent renal dialysis or severe cardiac angina. Very severe AMD causes a 63% decrement in quality of life, a decrease comparable to an individual with advanced prostatic cancer, suffering uncontrollable pain, or a severe stroke that leaves a person bedridden, incontinent and requiring constant nursing care [9]. Symptoms may develop slowly, especially if the disease presents in only one eye, but as the condition progresses, visual acuity will deteriorate resulting in gaps or dark spots (like a smudge on glasses) appearing in the individual's vision. In addition, words may disappear when reading and straight lines, such as door frames and lamp posts, may appear distorted or bent, and colours appear to fade. The subject may also find it difficult to adapt when moving from dark to light environments. Whilst AMD does not result in complete blindness, loss of central vision makes everyday activities extremely challenging. For example, it becomes hard to recognise faces, drive and read [1-3], and it has been reported that some individuals experience visual hallucinations [10].

Wet-AMD is the result of new blood vessels growing under the macula (neovascularisation), which during their formation, leak blood and fluid into the retina resulting in damage to the tissue [11]. Preventive efforts include exercising, eating well, and not smoking [12]. Antioxidant vitamins and minerals have a limited effect [13], and currently, there is no cure or treatment that can restore lost vision [1-3].

VEGF-A is a homodimeric glycoprotein produced and secreted by glial, ganglion and epithelial cells, including the retinal pigment epithelium (RPE) of the eye and by astrocytes [14,15]. There are multiple isoforms of VEGF-A resulting from alternative splicing of mRNA from a single, 8-exon VEGF-A, including the four principle forms VEGF-121, VEGF-165, VEGF-189 and VEGF-206, which display varying levels of heparin binding [14,15]. VEGF-A isoforms display key physiological roles in vascular development and are important for neuronal survival.

Current treatments targeting the vascular endothelial growth factor (VEGF) pathway include monoclonal antibodies or other VEGF neutralising fragments (ranibizumab, Lucentis®/bevacizumab, Avastin®/brolucizumab/Beovu®), DNA aptamer (pegaptanib/Macugen®), or recombinant VEGF capture protein (aflibercept; Eylea®), which have been shown to limit visual loss [16-19]. These treatments are now favoured over the more invasive laser coagulation or photodynamic earlier therapeutic options.

However, some patients show an intrinsic refractoriness to anti-VEGF therapy, with persistent fluid or recurrent exudation [20]. Additionally, advanced stage disease involves focal areas of retinal pigment epithelium (RPE) loss, subretinal or sub-RPE haemorrhage, as well as subretinal fibrosis. Current strategies targeting the VEGF pathway alone do not address these issues, and therefore, there is substantial room for improvement [20,21].

Furthermore, a recent multi-centre study examining the effects of anti-VEGF therapy in 1185 patients with wet-AMD noted that by year one, subretinal scars developed in around one third of eyes treated with anti-VEGF drugs, rising to around half the patients by the end of year two [21]. The induction of neovascularisation can result in the recruitment of inflammatory cells and fibroblasts, with the injury triggering the conversion of epithelial cells to myofibroblasts [22]. Together, these cells produce an epithelial-mesenchymal transition (EMT) where they can proliferate and cover the region of damage [22]. Chronic inflammation results in a persistent scar.

Macular fibrosis causes irreparable vision loss in neovascular age-related macular degeneration (nAMD), even with anti-vascular endothelial growth factor (VEGF) therapy. A factor implicated in the fibrosis pathophysiology is connective tissue growth factor/cellular communication network-2 (CTGF/CCN2) [23]. CTGF is a 38 kDa secreted, cysteine-rich protein first identified in human umbilical vein endothelial cells and is a member of the CCN family of growth factors [24-27]. CTGF is composed of four linked cysteine rich domains (I-IV) ranging from the insulin-like growth factor binding protein domain (IGFBP; domain I) at the N-terminus, a von Willebrand type-C repeat sequence (vWC; domain II), followed by a thrombospondin type1 repeat (TSP; domain III), and finally the C-terminal domain IV which has a cysteine knot motif [24-27].

CTGF has been shown to induce a contraction of fibroblast-populated collagen matrix and increase the components of the extracellular matrix, including collagen and fibronectin [26,27]. CTGF production and release, is induced by transforming growth factor beta (TGF-β), leading to fibrosis in conditions including biliary fibrosis and diabetic retinopathy [28-30]. No specific CTGF receptor responsible for the pro-fibrotic effects has been identified, however, CTGF has been shown to non-specifically bind to several other growth factor receptors, such as insulin growth factor-2 receptor [31], fibroblast growth factor receptor-2 [32], epidermal growth factor receptor [33], integrins [34-36], and the TrkA receptor [37]. CTGF is consistently found within fluid accumulating in the sub-retinal space after retinal detachment [38]. It appears for full activity, the full length CTGF must be cleaved by extracellular endopeptidases. Cleavage liberates the C-terminal portion of the protein, containing the domains III-IV, and the N-terminal portion, consisting of domains I-II. It is thought that the N-terminal domain can act as an inhibitor of CTGF activity [39].

There is growing evidence for the involvement of complement in the development of macula fibrosis. Increased plasma levels of C3a, C3d, Bb, and C5a have been observed in AMD patients [40-42]. In particular, in a study of 96 patients with nAMD the plasma levels of complement C3a, C4a and C5a were shown to be significantly higher than the control group and especially in the individuals with subretinal fibrosis [42]. In addition, higher plasma levels of C3a were detected in nAMD who responded partially to the anti-VEGF therapy.

Furthermore, the complement system (CS) is part of the innate immune system to defend against foreign pathogens such as microbes [43-45]. The complement system (CS) consists of three biochemical pathways: classical pathway (CP), lectin pathway (LP) and alternative pathway (AP), each of which has a distinct trigger. Whilst each pathway can be activated by separate components, the pathways converge to involve a key protein component called complement factor 3 (C3).

Activity of the AP can be modulated by Complement factor I (CFI) [46,47], which is also called the C3b/C4b inactivator because it cleaves the cell-bound or fluid phase of C3b and C4b. Another modulating factor is CD55 or decay-accelerating factor (DAF) [48-50], which is a membrane bound protein that also protects cells from complement-mediated lysis. CD55's primary function is to inactivate the C3 convertases by dissociating them into their constituent proteins [48]. A further modulatory protein called complement factor H-related protein-1 (CFHR1), which is a splice variant of complement factor H [51-53], is also involved in reducing complement activation by targeting the decay of the C3 convertase that is composed of C3b and factor B. Another factor capable of reducing complement activation is called Membrane Cofactor Protein or CD46, which is membrane bound. Once bound to C3b, factor H and CFHL-1 occupy the factor B binding site in C3b, accelerate the decay and prevent the formation of new C3 convertases. Furthermore, factor H, CFHL-1 and CD46 act as cofactors for the protease factor I that cleaves C3b to iC3b, as well as for C4b in the case of CD46. CFHR-1 does not mediate decay-accelerating of factor I cofactor activity. Like factor H, CFHR-1 binds to C3b and recognises self-surfaces by binding to glycosaminoglycan and inhibits the C5 convertase and terminal complement complex formation [53].

There is, therefore, a significant need for an improved therapy for the treatment of retinal disorders, such as AMD and, in particular, wet age-related macular degeneration (wet-AMD), which can neutralise VEGF and prevent inflammation and the development of subretinal fibrosis.

Using significant inventive endeavour, the inventors have carefully designed and constructed a novel genetic construct, which encodes an anti-VEGF protein and an anti-fibrotic protein, under the control of a single promoter, i.e. it is bicistronic. The promoter of the construct may be used to ensure that both the anti-VEGF protein and the anti-fibrotic protein are maximally expressed to reduce vascular leakage, fibrosis, scarring and inflammation.

Thus, according to a first aspect of the invention, there is provided a genetic construct comprising a promoter operably linked to a first coding sequence, which encodes an anti-VEGF protein, and a second coding sequence, which encodes an anti-fibrotic protein.

As described in the Examples, the inventors have surprisingly demonstrated that it is possible to combine the open reading frames (ORFs) which code for both an anti-VEGF protein and an anti-fibrotic protein, in a single genetic construct. This was especially challenging given the large size of each component. It could not have been predicted that it would have been possible to co-express both of these large proteins in physiologically useful concentrations from a single expression cassette, under the control of a single promoter, and for that expression cassette to be accommodated by an AAV vector (such as an rAAV-2 vector). Advantageously, with the construct of the invention, there is no need to inject a recombinant protein, as described in the prior art. Furthermore, in the prior art, it is still necessary to perform regular injections of protein into the eye, which is clearly disadvantageous, whereas the construct of the invention surprisingly only requires a single administration to achieve long-term therapeutic effect, thereby providing significant benefits to patients.

Preferably, the genetic construct of the invention comprises an expression cassette, one embodiment of which is shown in FIG. 2. As can be seen in FIG. 2, in one embodiment, the construct comprises the promoter, the first nucleotide sequence encoding the anti-VEGF protein, and the second nucleotide sequence encoding the anti-fibrotic protein. Thus, preferably the genetic construct and expression cassette may be referred to as being bicistronic.

The first and second coding sequences encoding the anti-VEGF protein and anti-fibrotic protein may be disposed in any order from the 5′ to the 3′. For example, in one embodiment, the coding sequence for the anti-VEGF protein is disposed 5′ of the coding sequence for the anti-fibrotic protein, preferably with a spacer sequencer therebetween. Alternatively, in another embodiment, the coding sequence for the anti-fibrotic protein may be disposed 5′ of the coding sequence for the anti-VEGF protein, preferably with a spacer sequence therebetween.

The promoter in the genetic construct of the first aspect may be any nucleotide sequence that is capable of inducing RNA polymerase to bind to and transcribe the first and second coding sequence.

The promoter may be constitutive or tissue-specific.

A suitable constitutive promoter may be the cytomegalovirus promoter. One embodiment of the nucleotide sequence (508 bp) encoding the cytomegalovirus (CMV) promoter is referred to herein as SEQ ID No: 1, as follows:

[SEQ ID No: 1]
CGTTACATAACTTACGGTAAATGGCCCGCCTGGCTGACCGCCCAACGACCCCCGCCCATTGACGTCAATAATGACGTA
TGTTCCCATAGTAACGCCAATAGGGACTTTCCATTGACGTCAATGGGTGGAGTATTTACGGTAAACTGCCCACTTGGC
AGTACATCAAGTGTATCATATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGCTATTACCATGGTGATGCG
GTTTTGGCAGTACATCAATGGGCGTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAACTCCGCCCCATTGACGCAAATGGG
CGGTAGGCGTGTACGGTGGGAGGTCTATATAAGCAGAGCT

In another embodiment, the promoter is preferably a truncated form of the CMV promoter. One embodiment of the nucleotide sequence (60 bp) encoding the truncated form of the CMV promoter is referred to herein as SEQ ID No: 2, as follows:

[SEQ ID No: 2]
AGGTAGGCGTGTACGGTGGGAGGTCTATATAAGCAGAGCTGGTTTAGTG
AACCGTCAGAT

In another embodiment, the promoter is a fusion of the cytomegalovirus (CMV) early enhancer element and the first intron of the chicken beta-actin gene (CAG). One embodiment of the nucleotide sequence (583 bp) encoding the cytomegalovirus early enhancer element and the first intron of chicken beta-actin gene is referred to herein as SEQ ID No: 3, as follows:

[SEQ ID No: 3]
CGTTACATAACTTACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGACTATTTACGGTAAACTGCCCACTTGGCAGTACA
TCAAGTGTATCATATGCCAAGTACGCCCCCTATTGACGTCAATGACGGTAAATGGCCCGCCTGGCATTATGCCCAGTACAT
GACCTTATGGGACTTTCCTACTTGGCAGTACATCTACGTATTAGTCATCGCTATTACCATGGTCGAGGTGAGCCCCACGTT
CTGCTTCACTCTCCCCATCTCCCCCCCCTCCCCACCCCCAATTTTGTATTTATTTATTTTTTAATTATTTTGTGCAGCGAT
GGGGGCGGGGGGGGGGGGGGGGCGCGCGCCAGGCGGGGGGGGGCGGGGCGAGGGGCGGGGCGGGGCGAGGCGGAGAGGTGC
GGCGGCAGCCAATCAGAGCGGCGCGCTCCGAAAGTTTCCTTTTATGGCGAGGCGGCGGCGGCGGCGGCCCTATAAAAAGCG
AAGCGCGCGGCGGGCG

A suitable tissue-specific promoter may be the vitelliform macular dystrophy protein-2 (VMD2) promoter (sometimes referred to as the bestrophin-1). Advantageously, this promoter restricts transgene expression to the RPE cells. One embodiment of the nucleotide sequence (2039 bp) encoding the VMD2 promoter is referred to herein as SEQ ID No: 4, as follows:

[SEQ ID No: 4]
AATTCTGTCATTTTACTAGGGTGATGAAATTCCCAAGCAACACCATCCTTTTCAGATAAGGGCACTGAGGCTGAGAGA
GGAGCTGAAACCTACCCGGCGTCACCACACACAGGTGGCAAGGCTGGGACCAGAAACCAGGACTGTTGACTGCAGCCC
GGTATTCATTCTTTCCATAGCCCACAGGGCTGTCAAAGACCCCAGGGCCTAGTCAGAGGCTCCTCCTTCCTGGAGAGT
TCCTGGCACAGAAGTTGAAGCTCAGCACAGCCCCCTAACCCCCAACTCTCTCTGCAAGGCCTCAGGGGTCAGAACACT
GGTGGAGCAGATCCTTTAGCCTCTGGATTTTAGGGCCATGGTAGAGGGGGTGTTGCCCTAAATTCCAGCCCTGGTCTC
AGCCCAACACCCTCCAAGAAGAAATTAGAGGGGCCATGGCCAGGCTGTGCTAGCCGTTGCTTCTGAGCAGATTACAAG
AAGGGACTAAGACAAGGACTCCTTTGTGGAGGTCCTGGCTTAGGGAGTCAAGTGACGGCGGCTCAGCACTCACGTGGG
CAGTGCCAGCCTCTAAGAGTGGGCAGGGGCACTGGCCACAGAGTCCCAGGGAGTCCCACCAGCCTAGTCGCCAGACCT
TCTGTGGGATCATCGGACCCACCTGGAACCCCACCTGTGAGTACAAGGTGCCCCAGGTGGACTGGGCTGGGGCTTTGA
GGCCTTCAGGGTTGGATGGCCATCTTGCGTATTTGTGTGGGATATGCACACACAGGCAGCACATGCGCAGGTGTGTGG
GCACCTGTGTGTCTGTGCAAATGCCCTGAGGTGGGAATGAGCTTGGTGTGCATCAGGCACAGCCAGCCAGTGTGGCTG
CAGCAAAACACACAGGGAAAGAATGGAGGGGGCATCAATCACTGCTTCAGTAAATTTTTATTGAGCGCCTTCTACGAG
AACACAAGAGGAGCTTCCATTCTGAGGAGGAAACAGGCAGGAAACAGGCAGATATCCTGTATAATTTCAAGTAGTGAT
AAGTGCTCTCTAGAAATATCAAGCAAGGTGAGGAGACACAGAGCACCGGTGGCAGTGGGGCTCTATTTCCAGGTTGGA
TGGTTGGGAACATCCTTTCTAAAGGGAACCTGGAGTGGGAAGGAACCATGCAGGTATCTCAGGAAGAGCTTCCTCCAG
GCAGGAAGATCAGCAGGTGGAAAGGCCCTGGAGCCACCATTCAGTAAACATCATTTGAGCATCTCTACCAGCTAGGTT
CCATTATGGGAATGGGAATATGGTGGTGGACAGGGCTGCCTGGTCCCTTCCATACTTCTCACACTAGGGTGGTTGAGA
GAGCTTGGGAGCTAACGAACAAGATGGGCTGAGAACACTGCCTAGCCCAGAGGACCTGAGCTTAGTGTGTAGACATTG
CTGCTGTTACTGCCTTTGTCATTGTATTATTTATTTATTTATTTATTTATTTTTAGACAGAGTTTTGCTCTTCTTACC
CAGGCTGGAGTGCAATGGCGTGATCTCAGCTCACTGCAACCTCCACCTCCTGGGTTCAAGCGATTCTCCTGCCTCAGC
CTCCTGAGTAGCTGGGATTACAGGCACCCGCACCACGCCTGGATAATTTTTTTGTATTTTTAGTAGAGACAGGGTTTC
ACCATGTTGGCCAGGCTGGTCTCGAACTCCTGACCTTAGGTGATCCACCTGCCTCGACTTCCCAAAGTGCTGGGATTA
TAGGCATGAGCCACTGCGCCCAGTGATTATAGAAAGTTAAAGGCACATGGCAATGCACACGCCTATCTACGTCTTCCC
TGCCAAAGCAAAGGGCAGCCTCTGGGCTCACTTTCTTGCGTTTCTACTTCCAAAAGGCAGTCAGAACTGGCAGGGCCT
TGGAGACCACTTCATCCACCTCCTAGGGTCCCTATGGGAGAGTTGAGGTCCAGAGCAGGGAAGGGTCCTGACAGGCTC
TGACCAGGGCCTCTGATCCCTACAAACCCCCAATCGGTGTCCCTCTCTACCAGGACCCAAGCCCACCTGCTGCAGCCC
ACTGCCTGGCC

In yet a further preferred embodiment, the promoter is a truncated form of the VMD2 promoter. One embodiment of the nucleotide sequence (623 bp) encoding the truncated form of the VMD2 promoter referred to herein as SEQ ID No: 5, as follows:

[SEQ ID No: 5]
AATTCTGTCATTTTACTAGGGTGATGAAATTCCCAAGCAACACCATCCTTTTCAGATAAGGGCACTGAGGCTGAGAGA
GGAGCTGAAACCTACCCGGCGTCACCACACACAGGTGGCAAGGCTGGGACCAGAAACCAGGACTGTTGACTGCAGCCC
GGTATTCATTCTTTCCATAGCCCACAGGGCTGTCAAAGACCCCAGGGCCTAGTCAGAGGCTCCTCCTTCCTGGAGAGT
TCCTGGCACAGAAGTTGAAGCTCAGCACAGCCCCCTAACCCCCAACTCTCTCTGCAAGGCCTCAGGGGTCAGAACACT
GGTGGAGCAGATCCTTTAGCCTCTGGATTTTAGGGCCATGGTAGAGGGGGTGTTGCCCTAAATTCCAGCCCTGGTCTC
AGCCCAACACCCTCCAAGAAGAAATTAGAGGGGCCATGGCCAGGCTGTGCTAGCCGTTGCTTCTGAGCAGATTACAAG
AAGGGACTAAGACAAGGACTCCTTTGTGGAGGTCCTGGCTTAGGGAGTCAAGTGACGGCGGCTCAGCACTCACGTGGG
CAGTGCCAGCCTCTAAGAGTGGGCAGGGGCACTGGCCACAGAGTCCCAGGGAGTCCCACCAGCCTAGTCGCCAGACC

In yet another preferred embodiment, the nucleotide sequence (462 bp) encoding the truncated form of the VMD2 promoter referred to herein as SEQ ID No: 6, as follows:

[SEQ ID No: 6]
TCATTCTTTCCATAGCCCACAGGGCTGTCAAAGACCCCAGGGCCTAGTCAGAGGCTCCTCCTTCCTGGAGAGTTCCTG
GCACAGAAGTTGAAGCTCAGCACAGCCCCCTAACCCCCAACTCTCTCTGCAAGGCCTCAGGGGTCAGAACACTGGTGG
AGCAGATCCTTTAGCCTCTGGATTTTAGGGCCATGGTAGAGGGGGTGTTGCCCTAAATTCCAGCCCTGGTCTCAGCCC
AACACCCTCCAAGAAGAAATTAGAGGGGCCATGGCCAGGCTGTGCTAGCCGTTGCTTCTGAGCAGATTACAAGAAGGG
ACTAAGACAAGGACTCCTTTGTGGAGGTCCTGGCTTAGGGAGTCAAGTGACGGCGGCTCAGCACTCACGTGGGCAGTG
CCAGCCTCTAAGAGTGGGCAGGGGCACTGGCCACAGAGTCCCAGGGAGTCCCACCAGCCTAGTCGCCAGACC

In another embodiment, the promoter is the human phosphoglycerate kinase-1 (PGK) promoter. One embodiment of the nucleotide sequence (500 bp) encoding the human PGK-1 promoter is referred to herein as SEQ ID No: 7, as follows:

[SEQ ID No: 7]
GGGTAGGGGAGGCGCTTTTCCCAAGGCAGTCTGGAGCATGCGCTTTAGCAGCCCCGCTGGGCACTTGGCGCTACACAAGTG
GCCTCTGGCCTCGCACACATTCCACATCCACCGGTAGGCGCCAACCGGCTCCGTTCTTTGGTGGCCCCTTCGCGCCACCTT
CTACTCCTCCCCTAGTCAGGAAGTTCCCCCCCGCCCCGCAGCTCGCGTCGTGCAGGACGTGACAAATGGAAGTAGCACGTC
TCACTAGTCTCGTGCAGATGGACAGCACCGCTGAGCAATGGAAGCGGGTAGGCCTTTGGGGCAGCGGCCAATAGCAGCTTT
GCTCCTTCGCTTTCTGGGCTCAGAGGCTGGGAAGGGGTGGGTCCGGGGGCGGGCTCAGGGGGGGGCTCAGGGGCGGGGCGG
GCGCCCGAAGGTCCTCCGGAGGCCCGGCATTCTGCACGCTTCAAAAGCGCACGTCTGCCGCGCTGTTCTCCTCTTCCTCAT
CTCCGGGCCTTTCG

In a further embodiment, the promoter is EF1α derived from the human EEF1A1 gene that expresses the alpha subunit of eukaryotic elongation factor 1. One embodiment of the nucleotide sequence (1182 bp) encoding the EF1α promoter is referred to herein as SEQ ID No: 8, as follows:

[SEQ ID No: 8]
GCTCCGGTGCCCGTCAGTGGGCAGAGCGCACATCGCCCACAGTCCCCGAGAAGTTGGGGGGAGGGGTCGGCAATTGAACCG
GTGCCTAGAGAAGGTGGCGCGGGGTAAACTGGGAAAGTGATGTCGTGTACTGGCTCCGCCTTTTTCCCGAGGGTGGGGGAG
AACCGTATATAAGTGCAGTAGTCGCCGTGAACGTTCTTTTTCGCAACGGGTTTGCCGCCAGAACACAGGTAAGTGCCGTGT
GTGGTTCCCGCGGGCCTGGCCTCTTTACGGGTTATGGCCCTTGCGTGCCTTGAATTACTTCCACGCCCCTGGCTGCAGTAC
GTGATTCTTGATCCCGAGCTTCGGGTTGGAAGTGGGTGGGAGAGTTCGAGGCCTTGCGCTTAAGGAGCCCCTTCGCCTCGT
GCTTGAGTTGAGGCCTGGCTTGGGCGCTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTGGCAAGATAGTCTTGTAAATGCGGGC
CAAGATCTGCACACTGGTATTTCGGTTTTTGGGGCCGCGGGCGGCGACGGGGCCCGTGCGTCCCAGCGCACATGTTCGGCG
AGGCGGGGCCTGCGAGCGCGGCCACCGAGAATCGGACGGGGGTAGTCTCAAGCTGGCCGGCCTGCTCTGGTGCCTGGCCTC
GCGCCGCCGTGTATCGCCCCGCCCTGGGCGGCAAGGCTGGCCCGGTCGGCACCAGTTGCGTGAGCGGAAAGATGGCCGCTT
CCCGGCCCTGCTGCAGGGAGCTCAAAATGGAGGACGCGGCGCTCGGGAGAGCGGGCGGGTGAGTCACCCACACAAAGGAAA
AGGGCCTTTCCGTCCTCAGCCGTCGCTTCATGTGACTCCACGGAGTACCGGGCGCCGTCCAGGCACCTCGATTAGTTCTCG
AGCTTTTGGAGTACGTCGTCTTTAGGTTGGGGGGAGGGGTTTTATGCGATGGAGTTTCCCCACACTGAGTGGGTGGAGACT
GAAGTTAGGCCAGCTTGGCACTTGATGTAATTCTCCTTGGAATTTGCCCTTTTTGAGTTTGGATCTTGGTTCATTCTCAAG
CCTCAGACAGTGGTTCAAAGTTTTTTTCTTCCATTTCAGGTGTCGTGA

In a further embodiment, the promoter is EF1α without the large intron. One embodiment of the nucleotide sequence (230 bp) encoding the EF1α promoter is referred to herein as SEQ ID No: 9, as follows:

[SEQ ID No: 9]
GCTCCGGTGCCCGTCAGTGGGCAGAGCGCACATCGCCCACAGTCCCCGAGAAGTTGGGGGGAGGGGTCGGCAATTGAACCG
GTGCCTAGAGAAGGTGGCGCGGGGTAAACTGGGAAAGTGATGTCGTGTACTGGCTCCGCCTTTTTCCCGAGGGTGGGGGAG
AACCGTATATAAGTGCAGTAGTCGCCGTGAACGTTCTTTTTCGCAACGGGTTTGCCGCCAGAACACAG

Therefore, preferably the promoter comprises a nucleic acid sequence substantially as set out in SEQ ID No: 1, 2, 3, 4, 5, 6, 7, 8, or 9, or a fragment or variant thereof.

The inventors have carefully considered the sequences of the anti-VEGF protein, and have produced several preferred embodiments of the protein that may be encoded by the first coding sequence in the genetic construct of the first aspect.

Preferably, the anti-VEGF protein is capable of capturing all soluble forms of VEGF, including VEGF-A, VEGF-B, VEGF-C, VEGF-D and/or placenta growth factor (PIGF). More preferably, the anti-VEGF protein specifically captures VEGF-A. Preferably, in this embodiment, the anti-VEGF protein captures all isoforms of VEGF-A, including VEGF-121, VEGF-145, VEGF-165, VEGF-183, VEGF-189 and/or VEGF-206.

Preferably, the anti-VEGF protein is an anti-VEGF antibody, or antigen-binding fragment thereof.

The antigen-binding fragment thereof may comprise or consist of any of the fragments selected from a group consisting of VH, VL, Fd, Fv, Fab, Fab′, scFv, F (ab′)₂ and Fc fragment, which bind VEGF. The antigen-binding fragment may include the complementarity Determining Regions (CDRs), which bind a VEGF epitope.

In one preferred embodiment, the anti-VEGF protein is a single chain variable fragment (SCVF). In other words, in this preferred embodiment, the anti-VEGF protein is an anti-VEGF single chain variable fragment.

Accordingly, in a first embodiment, the first coding sequence comprises a nucleotide sequence encoding an anti-VEGF single chain variable fragment (SCVF-1), capable of neutralising the most common isoforms of VEGF-A. Preferably, SCVF-1 comprises an amino acid sequence referred to herein as SEQ ID No: 10, or a fragment or variant thereof, as follows:

[SEQ ID No: 10]
DIQLTQSPSSLSASVGDRVTITCSASQDISNYLNWYQQKPGKAPKVLIYFTSSLHSGVPSRFSGSGSGTDFTLTISSL
QPEDFATYYCQQYSTVPWTFGQGTKVEIKRGGGGGSGGGGSGGGGSGGGGSEVQLVESGGGLVQPGGSLRLSCAASGY
DFTHYGMNWVRQAPGKGLEWVGWINTYTGEPTYAADFKRRFTFSLDTSKSTAYLQMNSLRAEDTAVYYCAKYPYYYGT
SHWYFDVWGQGTLVTVSS

Preferably, in this embodiment, the first coding sequence comprises a nucleotide sequence (756 bp) referred to herein as SEQ ID No: 11 or a fragment or variant thereof, as follows:

[SEQ ID No: 11]
GATATCCAGCTGACCCAGAGCCCTTCTAGCCTGTCCGCCTCTGTGGGCGACAGGGTGACCATCACATGTAGCGCCTCC
CAGGATATCTCCAACTATCTGAATTGGTACCAGCAGAAGCCAGGCAAGGCCCCCAAGGTGCTGATCTATTTCACCTCC
TCTCTGCACAGCGGCGTGCCATCCAGATTCTCTGGCAGCGGCTCCGGCACCGACTTTACCCTGACAATCAGCTCCCTG
CAGCCAGAGGATTTCGCCACATACTATTGCCAGCAGTACAGCACCGTGCCCTGGACATTTGGCCAGGGCACCAAGGTG
GAGATCAAGAGGGGAGGAGGAGGAGGATCTGGAGGAGGAGGCAGCGGCGGCGGCGGCTCCGGCGGCGGCGGCTCTGAG
GTGCAGCTGGTGGAGTCCGGAGGAGGCCTGGTGCAGCCAGGAGGATCTCTGAGGCTGAGCTGTGCCGCCTCCGGCTAT
GACTTCACCCACTACGGAATGAACTGGGTGCGCCAGGCACCTGGCAAGGGACTGGAGTGGGTGGGCTGGATCAATACC
TATACAGGCGAGCCAACCTACGCCGCCGACTTTAAGCGGCGGTTCACCTTCAGCCTGGATACCTCTAAGAGCACAGCC
TACCTGCAGATGAACTCCCTGAGGGCAGAGGACACCGCCGTGTACTATTGCGCCAAGTATCCTTACTATTACGGCACA
AGCCACTGGTACTTCGACGTGTGGGGACAGGGCACCCTGGTGACAGTGTCTAGC

In a second preferred embodiment, the first coding sequence comprises a nucleotide sequence encoding an anti-VEGF single chain variable fragment (SCVF-2), capable of neutralising the most common isoforms of VEGF-A. Preferably, SCVF-2 comprises an amino acid sequence referred to herein as SEQ ID No: 12, or a fragment or variant thereof, as follows:

[SEQ ID No: 12]
DIQMTQSPSSLSASVGDRVTITCSASQDISNYLNWYQQKPGKAPKVLIYFTSSLHSGVPSRFSGSGSGTDFTLTISSL
QPEDFATYYCQQYSTVPWTFGQGTKVEIKRGGGGGSGGGGSGGGGSGGGGSEVQLVESGGGLVQPGGSLRLSCAASGY
TFTNYGMNWVRQAPGKGLEWVGWINTYTGEPTYAADFKRRFTFSLDTSKSTAYLQMNSLRAEDTAVYYCAKYPHYYGS
SHWYFDVWGQGTLVTVSS

Preferably, in this second embodiment, the first coding sequence comprises a nucleotide sequence (756 bp) referred to herein as SEQ ID No: 13, or a fragment or variant thereof, as follows:

[SEQ ID No: 13]
GACATCCAGATGACCCAGAGCCCCAGCAGCCTGAGCGCCAGCGTGGGCGACCGCGTGACCATCACCTGCAGCGCCAGC
CAGGACATCAGCAACTACCTGAACTGGTACCAGCAGAAGCCCGGCAAGGCCCCCAAGGTGCTGATCTACTTCACCAGC
AGCCTGCACAGCGGCGTGCCCAGCCGCTTCAGCGGCAGCGGCAGCGGCACCGACTTCACCCTGACCATCAGCAGCCTG
CAGCCCGAGGACTTCGCCACCTACTACTGCCAGCAGTACAGCACCGTGCCCTGGACCTTCGGCCAGGGCACCAAGGTG
GAGATCAAGCGCGGCGGCGGCGGCGGCAGCGGCGGCGGCGGCAGCGGCGGCGGCGGCAGCGGCGGCGGCGGCAGCGAG
GTGCAGCTGGTGGAGAGCGGCGGCGGCCTGGTGCAGCCCGGCGGCAGCCTGCGCCTGAGCTGCGCCGCCAGCGGCTAC
ACCTTCACCAACTACGGCATGAACTGGGTGCGCCAGGCCCCCGGCAAGGGCCTGGAGTGGGTGGGCTGGATCAACACC
TACACCGGCGAGCCCACCTACGCCGCCGACTTCAAGCGCCGCTTCACCTTCAGCCTGGACACCAGCAAGAGCACCGCC
TACCTGCAGATGAACAGCCTGCGCGCCGAGGACACCGCCGTGTACTACTGCGCCAAGTACCCCCACTACTACGGCAGC
AGCCACTGGTACTTCGACGTGTGGGGCCAGGGCACCCTGGTGACCGTGAGCAGC

In a third preferred embodiment, the first coding sequence comprises a nucleotide sequence encoding an anti-VEGF single chain variable fragment (SCFV-3), capable of neutralising the most common isoforms of VEGF-A. Preferably, SCFV-3 comprises an amino acid sequence referred to herein as SEQ ID No: 14, or a fragment or variant thereof, as follows:

[SEQ ID No: 14]
MEIVMTQSPSTLSASVGDRVIITCQASEIIHSWLAWYQQKPGKAPKLLIYLASTLASGVPSRFSGSGSGAEFTLTISS
LQPDDFATYYCQNVYLASTNGANFGQGTKLTVLGGGGGSGGGGSGGGGSGGGGSEVQLVESGGGLVQPGGSLRLSCAA
SGYTFTNYGMNWVRQAPGKGLEWVGWINTYTGEPTYAADFKRRFTFSLDTSKSTAYLQMNSLRAEDTAVYYCAKYPHY
YGSSHWYFDVWGQGTLVTVSS

Preferably, in this third preferred embodiment, the first coding sequence comprises a nucleotide sequence (765 bp) referred to herein as SEQ ID No: 15, or a fragment or variant thereof, as follows:

[SEQ ID No: 15]
ATGGAGATCGTGATGACCCAGAGCCCCAGCACCCTGAGCGCCAGCGTGGGCGACCGCGTGATCATCACCTGCCAGGCC
AGCGAGATCATCCACAGCTGGCTGGCCTGGTACCAGCAGAAGCCCGGCAAGGCCCCCAAGCTGCTGATCTACCTGGCC
AGCACCCTGGCCAGCGGCGTGCCCAGCCGCTTCAGCGGCAGCGGCAGCGGCGCCGAGTTCACCCTGACCATCAGCAGC
CTGCAGCCCGACGACTTCGCCACCTACTACTGCCAGAACGTGTACCTGGCCAGCACCAACGGCGCCAACTTCGGCCAG
GGCACCAAGCTGACCGTGCTGGGCGGCGGCGGCGGCAGCGGCGGCGGCGGCAGCGGCGGCGGCGGCAGCGGCGGCGGC
GGCAGCGAGGTGCAGCTGGTGGAGAGCGGCGGCGGCCTGGTGCAGCCCGGCGGCAGCCTGCGCCTGAGCTGCGCCGCC
AGCGGCTACACCTTCACCAACTACGGCATGAACTGGGTGCGCCAGGCCCCCGGCAAGGGCCTGGAGTGGGTGGGCTGG
ATCAACACCTACACCGGCGAGCCCACCTACGCCGCCGACTTCAAGCGCCGCTTCACCTTCAGCCTGGACACCAGCAAG
AGCACCGCCTACCTGCAGATGAACAGCCTGCGCGCCGAGGACACCGCCGTGTACTACTGCGCCAAGTACCCCCACTAC
TACGGCAGCAGCCACTGGTACTTCGACGTGTGGGGCCAGGGCACCCTGGTGACCGTGAGCAGC

In a fourth preferred embodiment, the first coding sequence comprises a nucleotide sequence encoding an anti-VEGF single chain variable fragment (SCVF-4), capable of neutralising the most common isoforms of VEGF-A. Preferably, SCVF-4 comprises an amino acid sequence referred to herein as SEQ ID No: 16, or a fragment or variant thereof, as follows:

DIQMTQSPSSLSASVGDRVTITCSASQDISNYLNWYQQKPGKAPKVLIYFTSSLHSGVPSRFSGSGSGTDFTLTISSL
QPEDFATYYCQQYSTVPWTFGQGTKVEIKRGGGGGSGGGGSGGGGSGGGGSEVQLVESGGGLVQPGGSLRLSCTASGF
SLTDYYYMTWVRQAPGKGLEWVGFIDPDDDPYYATWAKGRFTISRDNSKNTLYLQMNSLRAEDTAVYYCAGGDHNSGW
GLDIWGQGTLVTVSS

### [SEQ ID No: 16]

Preferably, in this fourth embodiment, the first coding sequence comprises a nucleotide sequence (747 bp) referred to herein as SEQ ID No: 17, or a fragment or variant thereof, as follows:

[SEQ ID No: 17]
GACATCCAGATGACCCAGAGCCCCAGCAGCCTGAGCGCCAGCGTGGGCGACCGCGTGACCATCACCTGCAGCGCCAGC
CAGGACATCAGCAACTACCTGAACTGGTACCAGCAGAAGCCCGGCAAGGCCCCCAAGGTGCTGATCTACTTCACCAGC
AGCCTGCACAGCGGCGTGCCCAGCCGCTTCAGCGGCAGCGGCAGCGGCACCGACTTCACCCTGACCATCAGCAGCCTG
CAGCCCGAGGACTTCGCCACCTACTACTGCCAGCAGTACAGCACCGTGCCCTGGACCTTCGGCCAGGGCACCAAGGTG
GAGATCAAGCGCGGCGGCGGCGGCGGCAGCGGCGGCGGCGGCAGCGGCGGCGGCGGCAGCGGCGGCGGCGGCAGCGAG
GTGCAGCTGGTGGAGAGCGGCGGCGGCCTGGTGCAGCCCGGCGGCAGCCTGCGCCTGAGCTGCACCGCCAGCGGCTTC
AGCCTGACCGACTACTACTACATGACCTGGGTGCGCCAGGCCCCCGGCAAGGGCCTGGAGTGGGTGGGCTTCATCGAC
CCCGACGACGACCCCTACTACGCCACCTGGGCCAAGGGCCGCTTCACCATCAGCCGCGACAACAGCAAGAACACCCTG
TACCTGCAGATGAACAGCCTGCGCGCCGAGGACACCGCCGTGTACTACTGCGCCGGCGGCGACCACAACAGCGGCTGG
GGCCTGGACATCTGGGGCCAGGGCACCCTGGTGACCGTGAGCAGC

In a fifth preferred embodiment, the first coding sequence comprises a nucleotide sequence encoding an anti-VEGF protein (VEGF capture protein-1), a protein capable of neutralising all soluble forms of VEGF. Preferably, VEGF capture protein-1 comprises an amino acid sequence referred to herein as SEQ ID No: 18, or a fragment or variant thereof, as set out below:

[SEQ ID No: 18]
SDTGRPFVEMYSEIPEIIHMTEGRELVIPCRVTSPNITVTLKKFPLDTLIPDGKRIIWDSRKGFIISNATYKEIGLLT
CEATVNGHLYKTNYLTHRQTNTIIDVVLSPSHGIELSVGEKLVLNCTARTELNVGIDFNWEYPSSKHQHKKLVNRDLK
TQSGSEMKKFLSTLTIDGVTRSDQGLYTCAASSGLMTKKNSTFVRVHEKDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPG

Preferably, in this fifth embodiment, the first coding sequence comprises a nucleotide sequence (1293 bp) referred to herein as SEQ ID No: 19, or a fragment or variant thereof, as follows:

[SEQ ID No: 19]
AGCGACACCGGCCGCCCCTTCGTGGAGATGTACAGCGAGATCCCCGAGATCATCCACATGACCGAGGGCCGCGAGCTG
GTGATCCCCTGCCGCGTGACCAGCCCCAACATCACCGTGACCCTGAAGAAGTTCCCCCTGGACACCCTGATCCCCGAC
GGCAAGCGCATCATCTGGGACAGCCGCAAGGGCTTCATCATCAGCAACGCCACCTACAAGGAGATCGGCCTGCTGACC
TGCGAGGCCACCGTGAACGGCCACCTGTACAAGACCAACTACCTGACCCACCGCCAGACCAACACCATCATCGACGTG
GTGCTGAGCCCCAGCCACGGCATCGAGCTGAGCGTGGGCGAGAAGCTGGTGCTGAACTGCACCGCCCGCACCGAGCTG
AACGTGGGCATCGACTTCAACTGGGAGTACCCCAGCAGCAAGCACCAGCACAAGAAGCTGGTGAACCGCGACCTGAAG
ACCCAGAGCGGCAGCGAGATGAAGAAGTTCCTGAGCACCCTGACCATCGACGGCGTGACCCGCAGCGACCAGGGCCTG
TACACCTGCGCCGCCAGCAGCGGCCTGATGACCAAGAAGAACAGCACCTTCGTGCGCGTGCACGAGAAGGACAAGACC
CACACCTGCCCCCCCTGCCCCGCCCCCGAGCTGCTGGGCGGCCCCAGCGTGTTCCTGTTCCCCCCCAAGCCCAAGGAC
ACCCTGATGATCAGCCGCACCCCCGAGGTGACCTGCGTGGTGGTGGACGTGAGCCACGAGGACCCCGAGGTGAAGTTC
AACTGGTACGTGGACGGCGTGGAGGTGCACAACGCCAAGACCAAGCCCCGCGAGGAGCAGTACAACAGCACCTACCGC
GTGGTGAGCGTGCTGACCGTGCTGCACCAGGACTGGCTGAACGGCAAGGAGTACAAGTGCAAGGTGAGCAACAAGGCC
CTGCCCGCCCCCATCGAGAAGACCATCAGCAAGGCCAAGGGCCAGCCCCGCGAGCCCCAGGTGTACACCCTGCCCCCC
AGCCGCGACGAGCTGACCAAGAACCAGGTGAGCCTGACCTGCCTGGTGAAGGGCTTCTACCCCAGCGACATCGCCGTG
GAGTGGGAGAGCAACGGCCAGCCCGAGAACAACTACAAGACCACCCCCCCCGTGCTGGACAGCGACGGCAGCTTCTTC
CTGTACAGCAAGCTGACCGTGGACAAGAGCCGCTGGCAGCAGGGCAACGTGTTCAGCTGCAGCGTGATGCACGAGGCC
CTGCACAACCACTACACCCAGAAGAGCCTGAGCCTGAGCCCCGGC

In a sixth preferred embodiment, the first coding sequence comprises a nucleotide sequence encoding an anti-VEGF protein (VEGF capture protein-2), a protein capable of neutralising all soluble forms of VEGF. Advantageously, and preferably, VEGF capture protein-2 has a lower affinity for binding to human IgG-Fc-γreceptors I, II and III. Preferably, VEGF capture protein-2 comprises an amino acid sequence referred to herein as SEQ ID No: 20, or a fragment or variant thereof, as set out below:

[SEQ ID No: 20]
SDTGRPFVEMYSEIPEIIHMTEGRELVIPCRVTSPNITVTLKKFPLDTLIPDGKRIIWDSRKGFIISNATYKEIGLLT
CEATVNGHLYKTNYLTHRQTNTIIDVVLSPSHGIELSVGEKLVLNCTARTELNVGIDFNWEYPSSKHQHKKLVNRDLK
TQSGSEMKKFLSTLTIDGVTRSDQGLYTCAASSGLMTKKNSTFVRVHEKDKTHTCPPCPAPEAAGGPSVFLFPPKPKD
TLMISRTPEVTCVVVDVSHEDPEVKFNWYVDGVEVHNAKTKPREEQYNSTYRVVSVLTVLAQDWLNGKEYKCKVSNKA
LGAPIEKTISKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNAYTQKSLSLSPG

Preferably, in this sixth embodiment, the first coding sequence comprises a nucleotide sequence (1293 bp) referred to herein as SEQ ID No: 21, or a fragment or variant thereof, as follows:

[SEQ ID No: 21]
AGCGACACCGGCCGCCCCTTCGTGGAGATGTACAGCGAGATCCCCGAGATCATCCACATGACCGAGGGCCGCGAGCTG
GTGATCCCCTGCCGCGTGACCAGCCCCAACATCACCGTGACCCTGAAGAAGTTCCCCCTGGACACCCTGATCCCCGAC
GGCAAGCGCATCATCTGGGACAGCCGCAAGGGCTTCATCATCAGCAACGCCACCTACAAGGAGATCGGCCTGCTGACC
TGCGAGGCCACCGTGAACGGCCACCTGTACAAGACCAACTACCTGACCCACCGCCAGACCAACACCATCATCGACGTG
GTGCTGAGCCCCAGCCACGGCATCGAGCTGAGCGTGGGCGAGAAGCTGGTGCTGAACTGCACCGCCCGCACCGAGCTG
AACGTGGGCATCGACTTCAACTGGGAGTACCCCAGCAGCAAGCACCAGCACAAGAAGCTGGTGAACCGCGACCTGAAG
ACCCAGAGCGGCAGCGAGATGAAGAAGTTCCTGAGCACCCTGACCATCGACGGCGTGACCCGCAGCGACCAGGGCCTG
TACACCTGCGCCGCCAGCAGCGGCCTGATGACCAAGAAGAACAGCACCTTCGTGCGCGTGCACGAGAAGGACAAGACC
CACACCTGCCCCCCCTGCCCCGCCCCCGAGGCCGCCGGCGGCCCCAGCGTGTTCCTGTTCCCCCCCAAGCCCAAGGAC
ACCCTGATGATCAGCCGCACCCCCGAGGTGACCTGCGTGGTGGTGGACGTGAGCCACGAGGACCCCGAGGTGAAGTTC
AACTGGTACGTGGACGGCGTGGAGGTGCACAACGCCAAGACCAAGCCCCGCGAGGAGCAGTACAACAGCACCTACCGC
GTGGTGAGCGTGCTGACCGTGCTGGCCCAGGACTGGCTGAACGGCAAGGAGTACAAGTGCAAGGTGAGCAACAAGGCC
CTGGGCGCCCCCATCGAGAAGACCATCAGCAAGGCCAAGGGCCAGCCCCGCGAGCCCCAGGTGTACACCCTGCCCCCC
AGCCGCGACGAGCTGACCAAGAACCAGGTGAGCCTGACCTGCCTGGTGAAGGGCTTCTACCCCAGCGACATCGCCGTG
GAGTGGGAGAGCAACGGCCAGCCCGAGAACAACTACAAGACCACCCCCCCCGTGCTGGACAGCGACGGCAGCTTCTTC
CTGTACAGCAAGCTGACCGTGGACAAGAGCCGCTGGCAGCAGGGCAACGTGTTCAGCTGCAGCGTGATGCACGAGGCC
CTGCACAACGCCTACACCCAGAAGAGCCTGAGCCTGAGCCCCGGC

Therefore, in preferred embodiments, the first coding sequence comprises a nucleotide sequence substantially as set out in any one of SEQ ID No: 11, 13, 15, 17, 19 or 21, or a fragment or variant thereof. Preferably, the anti-VEGF protein comprises an amino acid sequence substantially as set out in SEQ ID No: 10, 12, 14, 16, 18 or 20, or a fragment or variant thereof.

It will be appreciated that the second coding sequence encodes an anti-fibrotic protein.

In one embodiment, the anti-fibrotic protein is an anti-complement protein. Preferably, the anti-complement protein is capable of neutralising or attenuating complement activation. Even more preferably, the anti-complement protein is capable of targeting the alternative pathway (AP) of the complement system. Preferably, the anti-complement protein minimally affects the classical pathway (CP) and/or the lectin pathway (LP) of the complement system. Preferably, the anti-complement protein does not target the classical pathway (CP) and/or the lectin pathway (LP) of the complement system.

Preferably, the anti-complement protein is capable of neutralising complement factors C3b and/or Bb. Accordingly, in this embodiment, the anti-complement protein is an anti-C3b or anti-Bb antibody, or antigen-binding fragment thereof.

The antigen-binding fragment thereof may comprise or consist of any of the fragments selected from a group consisting of VH, VL, Fd, Fv, Fab, Fab′, scFv, F (ab′)₂ and Fc fragment, which bind C3b and/or Bb. The antigen-binding fragment may include the complementarity Determining Regions (CDRs), which bind the C3b and/or Bb epitope.

Even more preferably, the anti-complement protein is a single chain variable fragment (SCVF). In other words, in this preferred embodiment, the anti-complement protein is an anti-C3b single chain variable fragment, or an anti-Bb single chain variable fragment.

Alternatively, in another preferred embodiment, the anti-complement protein is CD55 (decay accelerating factor; DAF). Preferably, the anti-complement protein is a non-membrane attached CD55. CD55 (DAF) destabilises the complement protein complexes, thereby reducing the activity of this biochemical pathway.

In another preferred embodiment, the anti-complement protein is complement factor H related protein-1 (CFHR1). Preferably, CFHR1 attenuates the complement system activity cascade.

In another preferred embodiment, the anti-complement protein is CD46. Preferably, in this embodiment, the anti-complement protein is the soluble (non-membrane associated) human complement regulatory protein CD46 (sCD46).

In another preferred embodiment, the anti-complement protein is a Complement Factor H-Like protein 1 (CFHL1), which is a splice variant of factor H that includes the regulatory domains and inhibits complement activation at the level of the central complement component C3 and beyond.

In one preferred embodiment, the amino acid sequence of anti-C3b single chain variable fragment is referred to herein as SEQ ID No: 22, or a fragment or variant thereof, as follows:

[SEQ ID No: 22]
DIQMTQSPSSLSASVGDRVTITCRASQDVSTAVAWYQQKPGKAPKLLIYSASFLYSGVPSRFSGSGSGTDFTLTISSL
QPEDFATYYCQQSYATLPTFEQGTKVEIKRGGGGGSGGGGSGGGGSGGGGSEVQLVESGGGLVQPGGSLRLSCAASGF
SFTSSSVSPGKGLEWVGLIYPYNGFNYYADSVKGRFTISADTSLQMNSLRAEDTAVYYCARNALYGSGGYYAMDYWGQ
GTLVTVSS

In a preferred embodiment, the nucleic acid sequence (726 bp) encoding the anti-C3b single chain variable fragment is referred to herein as SEQ ID No: 23, or a fragment or variant thereof, as follows:

[SEQ ID No: 23]
GACATCCAGATGACCCAGAGCCCCAGCAGCCTGAGCGCCAGCGTGGGCGACCGCGTGACCATCACCTGCCGCGCCAGC
CAGGACGTAAGCACCGCCGTGGCCTGGTACCAGCAGAAGCCCGGCAAGGCCCCCAAGCTGCTGATCTACAGCGCCAGC
TTCCTGTACAGCGGCGTGCCCAGCCGCTTCAGCGGCAGCGGCAGCGGCACCGACTTCACCCTGACCATCAGCAGCCTG
CAGCCCGAGGACTTCGCCACCTACTACTGCCAGCAGAGCTACGCCACCCTGCCCACCTTCGAGCAGGGCACCAAGGTG
GAGATCAAGCGCGGCGGCGGCGGCGGCAGCGGCGGCGGCGGCAGCGGCGGCGGCGGCAGCGGCGGCGGCGGCAGCGAG
GTGCAGCTGGTGGAGAGCGGCGGCGGCCTGGTGCAGCCCGGCGGCAGCCTGCGCCTGAGCTGCGCCGCCAGCGGCTTC
AGCTTCACCAGCAGCAGCGTGAGCCCCGGCAAGGGCCTGGAGTGGGTGGGCCTGATCTACCCCTACAACGGCTTCAAC
TACTACGCCGACAGCGTGAAGGGCCGCTTCACCATCAGCGCCGACACCAGCCTGCAGATGAACAGCCTGCGCGCCGAG
GACACCGCCGTGTACTACTGCGCCCGCAACGCCCTGTACGGCAGCGGCGGCTACTACGCCATGGACTACTGGGGCCAG
GGCACCCTGGTGACCGTGAGCAGC

In another embodiment, the amino acid sequence of anti-Bb single chain variable fragment is referred to herein as SEQ ID No: 24, or a fragment or variant thereof, as follows:

[SEQ ID No: 24]
DVQITQSPSYLAASPGETITINCRASKSISKYLAWYQDKPGKINKLLIYSGSTLQSGIPSRFSGSGSGTDFTLTISSL
EPEDFAMYYCQQHDEYPWTFGGGTKLEIKRGGGGGSGGGGSGGGGSGGGGSQVQLQQSGAELAKPGASVRMSCKASGY
TFTNYWIHWVKQRPGQGLEWIGYINPNTGYNDYNQKFKDKATLTADKSSSTVYMQLSSLTSEDSAVYYCARGGQLGLR
RAMDYWGQGTSVTVSS

In a preferred embodiment, the nucleic acid sequence (750 bp) encoding the anti-Bb single chain variable fragment is referred to herein as SEQ ID No: 25, or a fragment or variant thereof, as follows:

GACGTGCAGATCACCCAGAGCCCCAGCTACCTGGCCGCCAGCCCCGGCGAGACCATCACCATCAACTGCCGCGCCAGC
AAGAGCATCAGCAAGTACCTGGCCTGGTACCAGGACAAGCCCGGCAAGACCAACAAGCTGCTGATCTACAGCGGCAGC
ACCCTGCAGAGCGGCATCCCCAGCCGCTTCAGCGGCAGCGGCAGCGGCACCGACTTCACCCTGACCATCAGCAGCCTG
GAGCCCGAGGACTTCGCCATGTACTACTGCCAGCAGCACGACGAGTACCCCTGGACCTTCGGCGGCGGCACCAAGCTG
GAGATCAAGCGCGGCGGCGGCGGCGGCAGCGGCGGCGGCGGCAGCGGCGGCGGCGGCAGCGGCGGCGGCGGCAGCCAG
GTGCAGCTGCAGCAGAGCGGCGCCGAGCTGGCCAAGCCCGGCGCCAGCGTGCGCATGAGCTGCAAGGCCAGCGGCTAC
ACCTTCACCAACTACTGGATCCACTGGGTGAAGCAGCGCCCCGGCCAGGGCCTGGAGTGGATCGGCTACATCAACCCC
AACACCGGCTACAACGACTACAACCAGAAGTTCAAGGACAAGGCCACCCTGACCGCCGACAAGAGCAGCAGCACCGTG
TACATGCAGCTGAGCAGCCTGACCAGCGAGGACAGCGCCGTGTACTACTGCGCCCGCGGCGGCCAGCTGGGCCTGCGC
CGCGCCATGGACTACTGGGGCCAGGGCACCAGCGTGACCGTGAGCAGC

### [SEQ ID No: 25]

In another embodiment, the amino acid sequence of a soluble (non-membrane bound) form of CD55 (sCD55, also sometimes referred to as decay accelerating factor; DAF) is referred to herein as SEQ ID No: 26, or a fragment or variant thereof, as follows:

[SEQ ID No: 26]
DCGLPPDVPNAQPALEGRTSFPEDTVITYKCEESFVKIPGEKDSVICLKGSQWSDIEEFCNRSCEVPTRLNSASLKQP
YITQNYFPVGTVVEYECRPGYRREPSLSPKLTCLQNLKWSTAVEFCKKKSCPNPGEIRNGQIDVPGGILFGATISFSC
NTGYKLFGSTSSFCLISGSSVQWSDPLPECREIYCPAPPQIDNGIIQGERDHYGYRQSVTYACNKGFTMIGEHSIYCT
VNNDEGEWSGPPPECRGKSLTSKVPPTVQKPTTVNVPTTEVSPTSQKTTTKTTTPNAQATRSTPVSRTTKHFHETTPN
KGSGTTSG

In a preferred embodiment, the 960 nucleic acid sequence (960 bp) encoding a soluble (non-membrane-bound) form of CD55 (sCD55) (also sometimes known as decay accelerating factor; DAF) is referred to herein as SEQ ID No: 27, or a fragment or variant thereof, as follows:

[SEQ ID No: 27]
GACTGCGGCCTGCCCCCCGACGTGCCCAACGCCCAGCCCGCCCTGGAGGGCCGCACCAGCTTCCCCGAGGACACCGTG
ATCACCTACAAGTGCGAGGAGAGCTTCGTGAAGATCCCCGGCGAGAAGGACAGCGTGATCTGCCTGAAGGGCAGCCAG
TGGAGCGACATCGAGGAGTTCTGCAACCGCAGCTGCGAGGTGCCCACCCGCCTGAACAGCGCCAGCCTGAAGCAGCCC
TACATCACCCAGAACTACTTCCCCGTGGGCACCGTGGTGGAGTACGAGTGCCGCCCCGGCTACCGCCGCGAGCCCAGC
CTGAGCCCCAAGCTGACCTGCCTGCAGAACCTGAAGTGGAGCACCGCCGTGGAGTTCTGCAAGAAGAAGAGCTGCCCC
AACCCCGGCGAGATCCGCAACGGCCAGATCGACGTGCCCGGCGGCATCCTGTTCGGCGCCACCATCAGCTTCAGCTGC
AACACCGGCTACAAGCTGTTCGGCAGCACCAGCAGCTTCTGCCTGATCAGCGGCAGCAGCGTGCAGTGGAGCGACCCC
CTGCCCGAGTGCCGCGAGATCTACTGCCCCGCCCCCCCCCAGATCGACAACGGCATCATCCAGGGCGAGCGCGACCAC
TACGGCTACCGCCAGAGCGTGACCTACGCCTGCAACAAGGGCTTCACCATGATCGGCGAGCACAGCATCTACTGCACC
GTGAACAACGACGAGGGCGAGTGGAGCGGCCCCCCCCCCGAGTGCCGAGGCAAGAGCCTGACCAGCAAGGTGCCCCCC
ACCGTGCAGAAGCCCACCACCGTGAACGTGCCCACCACCGAGGTGAGCCCCACCAGCCAGAAGACCACCACCAAGACC
ACCACCCCCAACGCCCAGGCCACCCGCAGCACCCCCGTGAGCCGCACCACCAAGCACTTCCACGAGACCACCCCCAAC
AAGGGCAGCGGCACCACCAGCGGC

In a further embodiment, the amino acid sequence of human complement factor H related protein-1 (CFHR1) is referred to herein as SEQ ID No: 28, or a fragment or variant thereof, as follows:

[SEQ ID No: 28]
EATFCDFPKINHGILYDEEKYKPFSQVPTGEVFYYSCEYNFVSPSKSFWTRITCTEEGWSPTPKCLRLCFFPFVENGH
SESSGQTHLEGDTVQIICNTGYRLQNNENNISCVERGWSTPPKCRSTDTSCVNPPTVQNAHILSRQMSKYPSGERVRY
ECRSPYEMFGDEEVMCLNGNWTEPPQCKDSTGKCGPPPPIDNGDITSFPLSVYAPASSVEYQCQNLYQLEGNKRITCR
NGQWSEPPKCLHPCVISREIMENYNIALRWTAKQKLYLRTGESAEFVCKRGYRLSSRSHTLRTTCWDGKLEYPTCAKR

In a preferred embodiment, the nucleic acid sequence (936 bp) encoding human complement factor H related protein-1 (CFHR1) is referred to herein as SEQ ID No: 29, or a fragment or variant thereof, as follows:

[SEQ ID No: 29]
GAAGCAACATTTTGTGATTTTCCAAAAATAAACCATGGAATTCTATATGATGAAGAAAAATATAAGCCATTTTCCCAG
GTTCCTACAGGGGAAGTTTTCTATTACTCCTGTGAATATAATTTTGTGTCTCCTTCAAAATCATTTTGGACTCGCATA
ACATGCACAGAAGAAGGATGGTCACCAACACCAAAGTGTCTCAGACTGTGTTTCTTTCCTTTTGTGGAAAATGGTCAT
TCTGAATCTTCAGGACAAACACATCTGGAAGGTGATACTGTGCAAATTATTTGCAACACAGGATACAGACTTCAAAAC
AATGAGAACAACATTTCATGTGTAGAACGGGGCTGGTCCACCCCTCCCAAATGCAGGTCCACTGACACTTCCTGTGTG
AATCCGCCCACAGTACAAAATGCTCATATACTGTCGAGACAGATGAGTAAATATCCATCTGGTGAGAGAGTACGTTAT
GAATGTAGGAGCCCTTATGAAATGTTTGGGGATGAAGAAGTGATGTGTTTAAATGGAAACTGGACAGAACCACCTCAA
TGCAAAGATTCTACGGGAAAATGTGGGCCCCCTCCACCTATTGACAATGGGGACATTACTTCATTCCCGTTGTCAGTA
TATGCTCCAGCTTCATCAGTTGAGTACCAATGCCAGAACTTGTATCAACTTGAGGGTAACAAGCGAATAACATGTAGA
AATGGACAATGGTCAGAACCACCAAAATGCTTACATCCGTGTGTAATATCCCGAGAAATTATGGAAAATTATAACATA
GCATTAAGGTGGACAGCCAAACAGAAGCTTTATTTGAGAACAGGTGAATCAGCTGAATTTGTGTGTAAACGGGGATAT
CGTCTTTCATCACGTTCTCACACATTGCGAACAACATGTTGGGATGGGAAACTGGAGTATCCAACTTGTGCAAAAAGA

In another embodiment, the codon optimised nucleic acid sequence (936 bp) encoding human complement factor H related protein-1 (CFHR1) is referred to herein as SEQ ID No: 30, or a fragment or variant thereof, as follows:

[SEQ ID No: 30]
GAGGCCACCTTCTGCGACTTCCCCAAGATCAACCACGGCATCCTGTACGACGAGGAGAAGTACAAGCCCTTCAGCCAG
GTGCCCACCGGCGAGGTGTTCTACTACAGCTGCGAGTACAACTTCGTGAGCCCCAGCAAGAGCTTCTGGACCCGCATC
ACCTGCACCGAGGAGGGCTGGAGCCCCACCCCCAAGTGCCTGCGCCTGTGCTTCTTCCCCTTCGTGGAGAACGGCCAC
AGCGAGAGCAGCGGCCAGACCCACCTGGAGGGCGACACCGTGCAGATCATCTGCAACACCGGCTACCGCCTGCAGAAC
AACGAGAACAACATCAGCTGCGTGGAGCGCGGCTGGAGCACCCCCCCCAAGTGCCGCAGCACCGACACCAGCTGCGTG
AACCCCCCCACCGTGCAGAACGCCCACATCCTGAGCCGCCAGATGAGCAAGTACCCCAGCGGCGAGCGCGTGCGCTAC
GAGTGCCGCAGCCCCTACGAGATGTTCGGCGACGAGGAGGTGATGTGCCTGAACGGCAACTGGACCGAGCCCCCCCAG
TGCAAGGACAGCACCGGCAAGTGCGGCCCCCCCCCCCCCATCGACAACGGCGACATCACCAGCTTCCCCCTGAGCGTG
TACGCCCCCGCCAGCAGCGTGGAGTACCAGTGCCAGAACCTGTACCAGCTGGAGGGCAACAAGCGCATCACCTGCCGC
AACGGCCAGTGGAGCGAGCCCCCCAAGTGCCTGCACCCCTGCGTGATCAGCCGCGAGATCATGGAGAACTACAACATC
GCCCTGCGCTGGACCGCCAAGCAGAAGCTGTACCTGCGCACCGGCGAGAGCGCCGAGTTCGTGTGCAAGCGCGGCTAC
CGCCTGAGCAGCCGCAGCCACACCCTGCGCACCACCTGCTGGGACGGCAAGCTGGAGTACCCCACCTGCGCCAAGCGC

In a further embodiment, the amino acid sequence of a soluble (non-membrane-bound) form of CD46 (sCD46) is referred to herein as SEQ ID No: 31, or a fragment or variant thereof, as follows:

[SEQ ID No: 31]
CEEPPTFEAMELIGKPKPYYEIGERVDYKCKKGYFYIPPLATHTICDRNHTWLPVSDDACYRETCPYIRDPLNGQAVP
ANGTYEFGYQMHFICNEGYYLIGEEILYCELKGSVAIWSGKPPICEKVLCTPPPKIKNGKHTFSEVEVFEYLDAVTYS
CDPAPGPDPFSLIGESTIYCGDNSVWSRAAPECKVVKCRFPVVENGKQISGFGKKFYYKATVMFECDKGFYLDGSDTI
VCDSNSTWDPPVPKCLKVLPPSSTKPPALSHSVSTSSTTKSPASSASGPRPTYKPPVSNYPGYPKPEEGILDSLDV

In a preferred embodiment, the nucleic acid sequence (930 bp) encoding a soluble (non-membrane-bound) form of CD46 (sCD46) is referred to herein as SEQ ID No: 32, or a fragment or variant thereof, as follows:

[SEQ ID No: 32]
TGCGAGGAGCCCCCCACCTTCGAGGCCATGGAGCTGATCGGCAAGCCCAAGCCCTACTACGAGATCGGCGAGCGCGTG
GACTACAAGTGCAAGAAGGGCTACTTCTACATCCCCCCCCTGGCCACCCACACCATCTGCGACCGCAACCACACCTGG
CTGCCCGTGAGCGACGACGCCTGCTACCGCGAGACCTGCCCCTACATCCGCGACCCCCTGAACGGCCAGGCCGTGCCC
GCCAACGGCACCTACGAGTTCGGCTACCAGATGCACTTCATCTGCAACGAGGGCTACTACCTGATCGGCGAGGAGATC
CTGTACTGCGAGCTGAAGGGCAGCGTGGCCATCTGGAGCGGCAAGCCCCCCATCTGCGAGAAGGTGCTGTGCACCCCC
CCCCCCAAGATCAAGAACGGCAAGCACACCTTCAGCGAGGTGGAGGTGTTCGAGTACCTGGACGCCGTGACCTACAGC
TGCGACCCCGCCCCCGGCCCCGACCCCTTCAGCCTGATCGGCGAGAGCACCATCTACTGCGGCGACAACAGCGTGTGG
AGCCGCGCCGCCCCCGAGTGCAAGGTGGTGAAGTGCCGCTTCCCCGTGGTGGAGAACGGCAAGCAGATCAGCGGCTTC
GGCAAGAAGTTCTACTACAAGGCCACCGTGATGTTCGAGTGCGACAAGGGCTTCTACCTGGACGGCAGCGACACCATC
GTGTGCGACAGCAACAGCACCTGGGACCCCCCCGTGCCCAAGTGCCTGAAGGTGCTGCCCCCCAGCAGCACCAAGCCC
CCCGCCCTGAGCCACAGCGTGAGCACCAGCAGCACCACCAAGAGCCCCGCCAGCAGCGCCAGCGGCCCCCGCCCCACC
TACAAGCCCCCCGTGAGCAACTACCCCGGCTACCCCAAGCCCGAGGAGGGCATCCTGGACAGCCTGGACGTG

In a preferred embodiment, the amino acid sequence of the CFHL1 is referred to herein as SEQ ID No: 97, or a fragment or variant thereof, as follows:

[SEQ ID No: 97]
EDCNELPPRRNTEILTGSWSDQTYPEGTQAIYKCRPGYRSLGNVIMVCRKGEWVALNPLRKCQKRPCGHPGDTPFGTF
TLTGGNVFEYGVKAVYTCNEGYQLLGEINYRECDTDGWTNDIPICEVVKCLPVTAPENGKIVSSAMEPDREYHFGQAV
RFVCNSGYKIEGDEEMHCSDDGFWSKEKPKCVEISCKSPDVINGSPISQKIIYKENERFQYKCNMGYEYSERGDAVCT
ESGWRPLPSCEEKSCDNPYIPNGDYSPLRIKHRTGDEITYQCRNGFYPATRGNTAKCTSTGWIPAPRCTLKPCDYPDI
KHGGLYHENMRRPYFPVAVGKYYSYYCDEHFETPSGSYWDHIHCTQDGWSPAVPCLRKCYFPYLENGYNQNYGRKFVQ
GKSIDVACHPGYALPKAQTTVTCMENGWSPTPRCIR

In a preferred embodiment, the nucleic acid sequence (1278 bp) encoding CFHL1 is referred to herein as SEQ ID No: 98, or a fragment or variant thereof as follows:

[SEQ ID No: 98]
GAGGACTGCAACGAGCTGCCCCCCCGCCGCAACACCGAGATCCTGACCGGCAGCTGGAGCGACCAGACCTACCCCGAG
GGCACCCAGGCCATCTACAAGTGCCGCCCCGGCTACCGCAGCCTGGGCAACGTGATCATGGTGTGCCGCAAGGGCGAG
TGGGTGGCCCTGAACCCCCTGCGCAAGTGCCAGAAGCGCCCCTGCGGCCACCCCGGCGACACCCCCTTCGGCACCTTC
ACCCTGACCGGCGGCAACGTGTTCGAGTACGGCGTGAAGGCCGTGTACACCTGCAACGAGGGCTACCAGCTGCTGGGC
GAGATCAACTACCGCGAGTGCGACACCGACGGCTGGACCAACGACATCCCCATCTGCGAGGTGGTGAAGTGCCTGCCC
GTGACCGCCCCCGAGAACGGCAAGATCGTGAGCAGCGCCATGGAGCCCGACCGCGAGTACCACTTCGGCCAGGCCGTG
CGCTTCGTGTGCAACAGCGGCTACAAGATCGAGGGCGACGAGGAGATGCACTGCAGCGACGACGGCTTCTGGAGCAAG
GAGAAGCCCAAGTGCGTGGAGATCAGCTGCAAGAGCCCCGACGTGATCAACGGCAGCCCCATCAGCCAGAAGATCATC
TACAAGGAGAACGAGCGCTTCCAGTACAAGTGCAACATGGGCTACGAGTACAGCGAGCGCGGCGACGCCGTGTGCACC
GAGAGCGGCTGGCGCCCCCTGCCCAGCTGCGAGGAGAAGAGCTGCGACAACCCCTACATCCCCAACGGCGACTACAGC
CCCCTGCGCATCAAGCACCGCACCGGCGACGAGATCACCTACCAGTGCCGCAACGGCTTCTACCCCGCCACCCGGGGC
AACACCGCCAAGTGCACCAGCACCGGCTGGATCCCCGCCCCCCGCTGCACCCTGAAGCCCTGCGACTACCCCGACATC
AAGCACGGCGGCCTGTACCACGAGAACATGCGCCGCCCCTACTTCCCCGTGGCCGTGGGCAAGTACTACAGCTACTAC
TGCGACGAGCACTTCGAGACCCCCAGCGGCAGCTACTGGGACCACATCCACTGCACCCAGGACGGCTGGAGCCCCGCC
GTGCCCTGCCTGCGCAAGTGCTACTTCCCCTACCTGGAGAACGGCTACAACCAGAACTACGGCCGCAAGTTCGTGCAG
GGCAAGAGCATCGACGTGGCCTGCCACCCCGGCTACGCCCTGCCCAAGGCCCAGACCACCGTGACCTGCATGGAGAAC
GGCTGGAGCCCCACCCCCCGCTGCATCCGC

Alternatively, in another embodiment, the anti-fibrotic protein is capable of neutralising connective tissue growth factor (CTGF). Preferably, the anti-fibrotic protein is an anti-connective tissue growth factor (anti-CTGF) antibody, or antigen binding fragment thereof. Preferably, the anti-CTGF antibody, or antigen binding fragment thereof, is capable of neutralising connective tissue growth factor (CTGF).

The antigen-binding fragment thereof may comprise or consist of any of the fragments selected from a group consisting of VH, VL, Fd, Fv, Fab, Fab′, scFv, F (ab′)₂ and Fc fragment, which bind CTGF. The antigen-binding fragment may include the complementarity Determining Regions (CDRs), which bind the CTGF epitope.

Most preferably, the anti-CTGF antibody is an anti-CTGF single chain variable fragment (anti-CTGF SCVF).

As no specific receptor or binding site for CTGF has yet been identified, the inventors have utilised a single-chain variable fragment (SCVF) capable of neutralising the entire CTGF sequence (anti-CTGF SCVF-1), or an SCVF which can neutralise the C-terminal CTGF fragment (anti-CTGF SCVF-2).

The inventors have carefully considered the sequences of the SCVF capable of neutralising CTGF and have produced preferred embodiments of the protein that may be encoded by the second coding sequence in the genetic construct of the first aspect.

In one preferred embodiment, the amino acid sequence of anti-CTGF single chain variable fragment (anti-CTGF SCVF-1) is referred to herein as SEQ ID No: 33, or a fragment or variant thereof, as follows:

[SEQ ID No: 33]
DIQMTQSPSSLSASVGDRVTITCRASQGISSWLAWYQQKPEKAPKSLIYAASSLQSGVPSRFSGSGSGTDFTLTISSL
QPEDFATYYCQQYNSYPPTFGQGTKLEIKRGGGGGSGGGGSGGGGSGGGGSEGQLVQSGGGLVHPGGSLRLSCAGSGF
TFSSYGMHWVRQAPGKGLEWVSGIGTGGGTYSTDSVKGRFTISRDNAKNSLYLQMNSLRAEDMAVYYCARGDYYGSGS
FFDCWGQGTLVTVSS

In a preferred embodiment, the nucleic acid sequence (747 bp) encoding the anti-CTGF SCVF-1 is referred to herein as SEQ ID No: 34, or a fragment or variant thereof, as follows:

[SEQ ID No: 34]
GACATCCAGATGACCCAGAGCCCCAGCAGCCTGAGCGCCAGCGTGGGCGACCGCGTGACCATCACCTGCCGCGCCAGC
CAGGGCATCAGCAGCTGGCTGGCCTGGTACCAGCAGAAGCCCGAGAAGGCCCCCAAGAGCCTGATCTACGCCGCCAGC
AGCCTCCAGAGCGGCGTGCCCAGCCGCTTCAGCGGCAGCGGCAGCGGCACCGACTTCACCCTGACCATCAGCAGCCTC
CAGCCCGAGGACTTCGCCACCTACTACTGCCAGCAGTACAACAGCTACCCCCCCACCTTCGGCCAGGGCACCAAGCTG
GAGATCAAGCGCGGCGGCGGCGGCGGCAGCGGCGGCGGCGGCAGCGGCGGCGGCGGCAGCGGCGGCGGCGGCAGCGAG
GGCCAGCTGGTGCAGAGCGGCGGCGGCCTGGTGCACCCCGGCGGCAGCCTGCGCCTGAGCTGCGCCGGCAGCGGCTTC
ACCTTCAGCAGCTACGGCATGCACTGGGTGCGCCAGGCCCCCGGCAAGGGCCTGGAGTGGGTGAGCGGCATCGGCACC
GGCGGCGGCACCTACAGCACCGACAGCGTGAAGGGCCGCTTCACCATCAGCCGCGACAACGCCAAGAACAGCCTGTAC
CTCCAGATGAACAGCCTGCGCGCCGAGGACATGGCCGTGTACTACTGCGCCCGCGGCGACTACTACGGCAGCGGCAGC
TTCTTCGACTGCTGGGGCCAGGGCACCCTGGTGACCGTGAGCAGC

In another preferred embodiment, the amino acid sequence of anti-CTGF single chain variable fragment (anti-CTGF SCVF-2) is referred to herein as SEQ ID No: 35, or a fragment or variant thereof, as follows:

[SEQ ID No: 35]
AEVQLVESGGGVVRPGGSLRLSCAASGFTFDDYGMSWVRQAPGKGLEWVSYISSSGSTIYYADSVKGRFTISRDNAKN
SLYLQMNSLRAEDTAVYYCARGITETWGQGTLVTVSSGGGGSGGGGSGGSALQSVLTQPPSASGTPGQRVTISCSGSS
SNIGSNTVNWYQQLPGTAPKLLIYSNNQRPSGVPDRFSGSKSGTSASLAISGLQSEDEADYYCAAWDDSLGAVFGGGT
KLTVLGA

In a preferred embodiment, the nucleic acid sequence (723 bp) encoding the anti-CTGF SCVF-2 is referred to herein as SEQ ID No: 36, or a fragment or variant thereof, as follows:

[SEQ ID No: 36]
GCCGAGGTGCAGCTGGTGGAGAGCGGCGGCGGCGTGGTGCGGCCTGGCGGCAGCCTGCGCCTGAGCTGCGCCGCCAGC
GGCTTCACCTTCGACGACTACGGCATGAGCTGGGTGCGCCAGGCTCCAGGCAAGGGCCTGGAGTGGGTGAGCTACATC
AGCAGCAGCGGCAGCACCATCTACTACGCCGACAGCGTGAAGGGCCGCTTCACCATCAGCCGCGACAACGCCAAGAAC
AGCCTGTACCTGCAGATGAACAGCCTGCGCGCCGAGGACACCGCCGTGTACTACTGCGCACGAGGCATCACCGAGACC
TGGGGCCAGGGCACCCTGGTGACCGTGAGCAGCGGCGGCGGCGGCAGCGGCGGCGGCGGCAGCGGCGGCAGCGCCCTG
CAGAGCGTGCTGACCCAGCCACCCAGCGCCAGCGGCACCCCTGGCCAGCGCGTGACCATCAGCTGCAGCGGCAGCAGC
AGCAACATCGGCAGCAACACCGTGAACTGGTACCAGCAGCTGCCCGGCACCGCGCCTAAGCTGCTGATCTACAGCAAC
AACCAGCGCCCCAGCGGCGTGCCCGACCGCTTCAGCGGCAGCAAGAGCGGCACCAGCGCCAGCCTGGCCATCAGCGGC
CTGCAGAGCGAGGACGAGGCCGACTACTACTGCGCCGCCTGGGACGACAGCCTGGGCGCCGTGTTCGGCGGCGGCACC
AAGCTGACCGTGCTGGGCGCC

Therefore, in preferred embodiments, the second coding sequence comprises a nucleotide sequence substantially as set out in any one of SEQ ID No: 23, 25, 27, 29, 30, 32, 34, 36 or 98, or a fragment or variant thereof. Preferably, the anti-complement protein comprises an amino acid sequence substantially as set out in SEQ ID No: 22, 24, 26, 28, 31, 33, 35 or 97, or a fragment or variant thereof.

Many gene therapy constructs expressing two or more genes that are presented in the scientific literature have either (i) dual promoters to separately drive expression of two or more genes, or (ii) an internal ribosome entry site (IRES) to link the genes, such as that from the encephalomyocarditis virus (EMCV), to enable translation of the genes from a single transcript driven by a single promoter within recombinant viral vectors. However, the efficiency of IRES-dependent translation varies dramatically in different cells and tissues, and IRES-dependent translation can be significantly lower than cap-dependent translation, meaning that there is often lower expression of genes downstream of an IRES when compared to the gene in position one of the expression cassette. Moreover, the limited coding capacity of rAAV vectors (generally <5 kb) prevents the incorporation of large genes/ORFs, such as a coding sequence for one of the anti-VEGF protein and anti-fibrotic protein, using dual promoters and/or IRES linkers (for which the EMCV IRES is 553 nucleotides in length).

Accordingly, in a preferred embodiment, the genetic construct comprises a spacer sequence disposed between the first and second coding sequences. For example, see FIG. 3, in which the spacer sequence (v2A) is disposed between the first and second coding sequences. This spacer sequence encodes a peptide spacer that is configured to produce the anti-VEGF protein and anti-fibrotic protein as separate molecules. This is possible as the spacer is configured to skip the linear ribosomal sequence transcription to produce the separate molecules or peptides. It will be appreciated that the separate molecules are active.

Preferably, the spacer sequence comprises and encodes a viral peptide spacer sequence, most preferably a viral-2A peptide spacer sequence. In one embodiment, this viral-2A peptide spacer sequence comprises a F2A, E2A, T2A or P2A sequence.

Preferably, the viral-2A peptide sequence connects the first coding sequence to the second coding sequence. This enables the construct to overcome the size restrictions that occur with expression in various vectors and enables expression of all of the peptides encoded by the construct of the first aspect to occur under control of a single promoter, as a single mRNA transcript.

Thus, in one embodiment, following the transcription of the single mRNA transcript encoding the sequences of the anti-VEGF protein, the viral-2A peptide, and the anti-fibrotic protein, translational skipping may occur at the viral-2A peptide sequence between the terminal glycine-proline of the viral-2A peptide. This translational skipping will thereby generate two proteins, i.e. the anti-VEGF protein and the anti-fibrotic protein (see FIG. 3).

The inventors have generated four embodiments of the spacer sequence. One important section of the peptide spacer sequence, which is common to all embodiments described herein, is the C-terminus.

In one embodiment, the peptide spacer sequence is P2A. Preferably, the P2A peptide spacer sequence encodes an amino acid sequence referred to herein as SEQ ID No: 37, or a fragment or variant thereof, as follows:

[SEQ ID No: 37]
ATNFSLLKQAGDVEENPGP

Preferably, the digestion or cut site of the peptide spacer sequence is disposed between the terminal glycine and end proline in SEQ ID No: 37.

In this first embodiment, the P2A peptide spacer sequence comprises a nucleotide sequence (57 bp) referred to herein as SEQ ID No: 38, or a fragment or variant thereof, as follows:

[SEQ ID No: 38]
GCCACCAACTTCAGCCTGCTGAAGCAGGCCGGCGACGTGGAGGAGAACC
CCGGCCCC

In a second embodiment, the peptide spacer sequence is E2A. Preferably, the E2A peptide spacer sequence encodes an amino acid sequence referred to herein as SEQ ID No: 39, or a fragment or variant thereof, as follows:

[SEQ ID No: 39]
QCTNYALLKLAGDVESNPGP

Preferably, the digestion or cut site of the peptide spacer sequence is disposed between the terminal glycine and end proline in SEQ ID No: 39.

In this second embodiment, the E2A peptide spacer sequence comprises a nucleotide sequence (60 bp) referred to herein as SEQ ID No: 40, or a fragment or variant thereof, as follows:

[SEQ ID No: 40]
CAGTGCACCAACTACGCCCTGCTGAAGCTGGCCGGCGACGTGGAGAGCA
ACCCCGGCCCC

In a third embodiment, the peptide spacer sequence is T2A. Preferably, the T2A peptide spacer sequence encodes an amino acid sequence referred to herein as SEQ ID No: 41, or a fragment or variant thereof, as follows:

[SEQ ID No: 41]
EGRGSLLTCGDVEENPGP

Preferably, the digestion or cut site of the peptide spacer sequence is disposed between the terminal glycine and end proline in SEQ ID No: 41.

In this third embodiment, the T2A peptide spacer sequence comprises a nucleotide sequence (54 bp) referred to herein as SEQ ID No: 42, or a fragment or variant thereof, as follows:

[SEQ ID No: 42]
GAGGGCCGCGGCAGCCTGCTGACCTGCGGCGACGTGGAGGAGAACCCCG
GCCCC

In a fourth preferred embodiment, the peptide spacer sequence is F2A. Preferably, the F2A peptide spacer sequence encodes an amino acid sequence referred to herein as SEQ ID No: 43 or a fragment or variant thereof, as follows:

[SEQ ID No: 43]
VKQTLNFDLLKLAGDVESNPGP

Preferably, the digestion or cut site of the peptide spacer sequence is disposed between the terminal glycine and end proline in SEQ ID No: 43.

In this fourth embodiment, the F2A peptide spacer sequence comprises a nucleotide sequence (66 bp) referred to herein as SEQ ID No: 44, or a fragment or variant thereof, as follows:

[SEQ ID No: 44]
GTGAAGCAGACCCTGAACTTCGACCTGCTGAAGCTGGCCGGCGACGTGG
AGAGCAACCCCGGCCCC

Therefore, in one preferred embodiment, the peptide spacer sequence comprises a nucleotide sequence substantially as set out in any one of SEQ ID No: 38, 40, 42 or 44, or a fragment or variant thereof. Preferably, the peptide spacer sequence encodes an amino acid sequence substantially as set out in SEQ ID No: 37, 39, 41 or 43, or a fragment or variant thereof.

After translational skipping, the viral-2A peptide sequence remains fused to the C-terminus of the upstream protein (such as the anti-VEGF protein), whilst the proline remains fused to the N-terminus of the downstream protein (such as the anti-fibrotic protein). This poses an immunogenicity risk and may potentially interfere with the intracellular signalling capability. Therefore, the inventors have introduced an enzyme cleavage coding sequence directly upstream of the viral-2A peptide sequence, such that the remaining viral-2A peptide sequence from both the encoded proteins (i.e. the anti-VEGF protein and the anti-fibrotic protein) is removed. The introduction of the enzyme cleavage site has the effect of removing the viral-2A peptide either intracellularly prior to release of the secreted proteins, in the case of the enzyme furin, or shortly after the proteins have been secreted from the target (retinal) cells, in the case of the enzyme recognition sites for matrix metalloprotein-2 (MMP-2) or renin. MMP-2 and furin are enzymes that are known to be secreted from Müller glial cells, and are therefore available to cut and remove the viral-2A peptide sequence from the anti-VEGF protein and the anti-fibrotic protein within the neural retina following secretion.

Accordingly, in one embodiment, the construct further comprises a viral-2A removal sequence. Preferably, the viral-2A removal sequence is disposed 5′ of the viral-2A sequence. Preferably, the viral-2A removal sequence is separated from the viral-2A sequence by a linker sequence comprising a tripeptide glycine-serine-glycine sequence (G-S-G).

The inventors have introduced a furin recognition sequence to enzymatically remove the viral-2A peptide sequence from the C-terminal of the proteins. Accordingly, in one embodiment, the viral-2A removal sequence is a furin recognition sequence.

Currently, the furin recognition sequence is generally recognised as comprising three or four basic amino acids (arginine or lysine) with an optional non-basic amino acid at position 2, and is cleaved by the enzyme furin after the last basic amino acid. However, using various plasmid constructs, the inventors determined that this basic furin recognition sequence does not always result in enzymatic activity and separation of the viral-2A sequence. As such, the inventors have generated a preferred furin recognition sequence for use in the genetic construct of the invention.

Accordingly, in a preferred embodiment, the genetic construct comprises a viral-2A removal sequence encoding an amino acid sequence referred to herein as SEQ ID No: 45, or a fragment or variant thereof, in which: B=basic amino acid, X=hydrophilic amino acid, and S=serine, as follows:

BB (X) BBS

### [SEQ ID No: 45]

Preferably, the hydrophilic amino acid (X) is either serine (S) or threonine (T). Accordingly, in one embodiment, the viral-2A removal sequence encodes an amino acid sequence substantially as set out in SEQ ID No: 45, or a fragment or variant thereof.

In one embodiment, the viral-2A removal sequence encodes an amino acid sequence referred to herein as SEQ ID No: 46, or a fragment or variant thereof, as follows:

[SEQ ID No: 46]
RRSKRSGSG

In this first embodiment, the viral-2A removal sequence comprises a nucleotide sequence referred to herein as SEQ ID No: 47, or a fragment or variant thereof, as follows:

[SEQ ID No: 47]
CGCCGCAGCAAGCGCAGCGGCAGCGGC

In a second embodiment, the viral-2A removal sequence encodes an amino acid sequence referred to herein as SEQ ID No: 48, or a fragment or variant thereof, as follows:

[SEQ ID No: 48]
RRTKRSGSG

In this second embodiment, the viral-2A removal sequence comprises a nucleotide sequence referred to herein as SEQ ID No: 49, or a fragment or variant thereof, as follows:

[SEQ ID No: 49]
CGCCGCACCAAGCGCAGCGGCAGCGGC

In a third embodiment, the viral-2A removal sequence encodes an amino acid sequence referred to herein as SEQ ID No: 50, or a fragment or variant thereof, as follows:

[SEQ ID No: 50]
RVRRGSG

In this third embodiment, the viral-2A removal sequence comprises a nucleotide sequence referred to herein as SEQ ID No: 51, or a fragment or variant thereof, as follows:

[SEQ ID No: 51]
CGCGTGCGCCGCGGCAGCGGC

Therefore, in one embodiment, the viral-2A removal sequence comprises a nucleotide sequence substantially as set out in either SEQ ID No: 47, 49 or 51, or a fragment or variant thereof. Preferably, the viral-2A removal sequence encodes an amino acid sequence substantially as set out in SEQ ID No: 46, 48 or 50, or a fragment or variant thereof.

Alternatively, in another embodiment, the viral-2A removal sequence is a gelatinase MMP-2 recognition sequence. Preferably, in this embodiment, the viral-2A removal sequence encodes the amino acid sequence GPQGIAGQ [SEQ ID No: 52], GPLGIAGA [SEQ ID No: 53] or GPQGLLGQ [SEQ ID No: 54], or a fragment or variant thereof. Cleavage preferably occurs after the second glycine residue.

The inventors have generated a preferred amino acid sequence referred to herein as SEQ ID No: 55, which comprises a gelatinase MMP-2 recognition sequence and the tripeptide GSG linker sequence.

Accordingly, in one embodiment, the viral-2A removal sequence encodes an amino acid sequence referred to herein as SEQ ID No: 55, or a fragment or variant thereof, as follows:

[SEQ ID No: 55]
GPQGIAGQGSG

In this embodiment, the viral-2A removal sequence comprises a nucleotide sequence referred to herein as SEQ ID No: 56, or a fragment or variant thereof, as follows:

[SEQ ID No: 56]
GGCCCCCTGGGCATCGCCGGCCAGGGCAGCGGC

Therefore, in one embodiment, the viral-2A removal sequence comprises a nucleotide sequence substantially as set out in SEQ ID No: 56, or a fragment or variant thereof. Preferably, the viral-2A removal sequence encodes an amino acid sequence substantially as set out in SEQ ID No: 55, or a fragment or variant thereof.

Alternatively, in another embodiment, the viral-2A removal sequence is a renin recognition sequence. Preferably, in this embodiment, the viral-2A removal sequence encodes the amino acid sequence HPFHLVYS [SEQ ID No: 57] or HPFHLLVYS [SEQ ID No: 58], or a fragment or variant thereof. Cleavage preferably occurs after the leucine residue(s).

The inventors have generated a preferred amino acid sequence referred to herein as SEQ ID No: 59, which comprises a renin recognition sequence and the tripeptide GSG linker sequence.

Accordingly, in one embodiment, the viral-2A removal sequence encodes an amino acid sequence referred to herein as SEQ ID No: 59, or a fragment or variant thereof, as follows:

[SEQ ID No: 59]
HPFHLLVYSGSG

In this embodiment, the viral-2A removal sequence comprises a nucleotide sequence referred to herein as SEQ ID No: 60, or a fragment or variant thereof, as follows:

[SEQ ID No: 60]
CGCCCCTTCCACCTGCTGGTCATCCACGGCAGCGGC

Therefore, in one embodiment, the viral-2A removal sequence comprises a nucleotide sequence substantially as set out in SEQ ID No: 60, or a fragment or variant thereof. Preferably, the viral-2A removal sequence encodes an amino acid sequence substantially as set out in SEQ ID No: 59, or a fragment or variant thereof.

As illustrated in FIG. 2, the expression cassette further comprises a sequence encoding Hepatitis Virus Post-transcriptional Regulatory Element (WPRE), a sequence encoding a poly-A tail, and left and right-hand Inverted Terminal Repeat sequences (ITRs). Preferably, the genetic construct comprises a nucleotide sequence encoding Woodchuck Hepatitis Virus (WHP) Post-transcriptional Regulatory Element (WPRE), which enhances the expression of the transgenes, i.e. the anti-VEGF protein and the anti-fibrotic protein. Preferably, the WPRE coding sequence is disposed 3′ of the transgene coding sequence.

One embodiment of the Woodchuck Hepatitis Virus Post-transcriptional Regulatory Element (WPRE) is 592 nucleotides long, including gamma-alpha-beta elements, and is referred to herein as SEQ ID No: 61, as follows:

[SEQ ID No: 61]
AATCAACCTCTGGATTACAAAATTTGTGAAAGATTGACT
GGTATTCTTAACTATGTTGCTCCTTTTACGCTATGTGGA
TACGCTGCTTTAATGCCTTTGTATCATGCTATTGCTTCC
CGTATGGCTTTCATTTTCTCCTCCTTGTATAAATCCTGG
TTGCTGTCTCTTTATGAGGAGTTGTGGCCCGTTGTCAGG
CAACGTGGCGTGGTGTGCACTGTGTTTGCTGACGCAACC
CCCACTGGTTGGGGCATTGCCACCACCTGTCAGCTCCTT
TCCGGGACTTTCGCTTTCCCCCTCCCTATTGCCACGGCG
GAACTCATCGCCGCCTGCCTTGCCCGCTGCTGGACAGGG
GCTCGGCTGTTGGGCACTGACAATTCCGTGGTGTTGTCG
GGGAAGCTGACGTCCTTTCCATGGCTGCTCGCCTGTGTT
GCCACCTGGATTCTGCGCGGGACGTCCTTCTGCTACGTC
CCTTCGGCCCTCAATCCAGCGGACCTTCCTTCCCGCGGC
CTGCTGCCGGCTCTGCGGCCTCTTCCGCGTCTTCGCCTT
CGCCCTCAGACGAGTCGGATCTCCCTTTGGGCCGCCTCC
CCGCCTG

Preferably, the WPRE comprises a nucleic acid sequence substantially as set out in SEQ ID No: 61, or a fragment or variant thereof.

However, in a preferred embodiment, a truncated WPRE is used, which is 247 nucleotides long due to deletion of the β-element, and which is referred to herein as SEQ ID No: 62, as follows:

[SEQ ID No: 62]
AATCAACCTCTGGATTACAAAATTTGTGAAAGATTGACT
GGTATTCTTAACTATGTTGCTCCTTTTACGCTATGTGGA
TACGCTGCTTTAATGCCTTTGTATCATGCTATTGCTTCC
CGTATGGCTTTCATTTTCTCCTCCTTGTATAAATCCTGG
TTAGTTCTTGCCACGGCGGAACTCATCGCCGCCTGCCTT
GCCCGCTGCTGGACAGGGGCTCGGCTGTTGGGCACTGAC
AATTCCGTGGTGT

Preferably, therefore, the truncated WPRE comprises a nucleic acid sequence substantially as set out in SEQ ID No: 62, or a fragment or variant thereof.

Advantageously, the truncated WPRE sequence used in the construct saves about 300 nucleotides in total without negatively impacting on transgene expression. Preferably, therefore, the WPRE comprises a nucleic acid sequence substantially as set out in SEQ ID No: 62, or a fragment or variant thereof.

Preferably, the genetic construct comprises a nucleotide sequence encoding a poly-A tail. Preferably, the poly-A tail coding sequence is disposed 3′ of the transgene coding sequence, and preferably 3′ of the WPRE coding sequence. The polyA tail is important for the nuclear export, translation, and stability of mRNA. The tail is shortened over time, and, when it is short enough, the mRNA is enzymatically degraded.

Preferably, the poly-A tail comprises the simian virus-40 poly-A 224 nucleotide sequence. One embodiment of the poly-A tail is referred to herein as SEQ ID No: 63, as follows:

[SEQ ID No: 63]
AGCAGACATGATAAGATACATTGATGAGTTTGGACAAAC
CACAACTAGAATGCAGTGAAAAAAATGCTTTATTTGTGA
AATTTGTGATGCTATTGCTTTATTTGTAACCATTATAAG
CTGCAATAAACAAGTTAACAACAACAATTGCATTCATTT
TATGTTTCAGGTTCAGGGGGAGGTGTGGGAGGTTTTTTA
AAGCAAGTAAAACCTCTACAAATGTGGTA

In another embodiment, the poly-A tail comprises a 169 nucleotide sequence polyA component, which is referred to herein as SEQ ID No: 64, as follows:

[SEQ ID No: 64]
TTCGAGCAACTTGTTTATTGCAGCTTATAATGGTTACAA
ATAAAGCAATAGCATCACAAATTTCACAAATAAAGCATT
TTTTTCACTGCATTCTAGTTGTGGTTTGTCCAAACTCAT
CAATGTATCTTATCATGTCTGGATCGTCTAGCATCGAAG
ATCCCCCGATCTG

In a further embodiment, the poly-A tail comprises the bovine growth hormone poly-A 225 nucleotide sequence, which is referred to herein as SEQ ID No: 99, as follows:

[SEQ ID No: 99]
CTGTGCCTTCTAGTTGCCAGCCATCTGTTGTTTGCCCCT
CCCCCGTGCCTTCCTTGACCCTGGAAGGTGCCACTCCCA
CTGTCCTTTCCTAATAAAATGAGGAAATTGCATCGCATT
GTCTGAGTAGGTGTCATTCTATTCTGGGGGGTGGGGTGG
GGCAGGACAGCAAGGGGGAGGATTGGGAAGACAATAGCA
GGCATGCTGGGGATGCGGTGGGCTCTATGG

Preferably, therefore, the poly-A tail comprises a nucleic acid sequence substantially as set out in SEQ ID No: 63, 64 or 99, or a fragment or variant thereof.

Preferably, the genetic construct comprises left and/or right Inverted Terminal Repeat sequences (ITRs). Preferably, each ITR is disposed at the 5′ and/or 3′ end of the construct. An ITR can be specific to a virus (e.g. AAV or lentivirus) serotype, and can be any sequence, so long as it forms a hairpin loop in its secondary structure.

The DNA sequence of one embodiment (left ITR sequence taken from a commercially available recombinant AAV genome plasmid) of the ITR is represented herein as SEQ ID No: 65, as follows:

[SEQ ID No: 65]
CGCGCTCGCTCGCTCACTGAGGCCGCCCGGGCAAAGCCC
GGGCGTCGGGCGACCTTTGGTCGCCCGGCCTCAGTGAGC
GAGCGAGCGCGCAGAGAGGGAGTGGCCAACTCCATCACT
AGGGGTTCCTT

The DNA sequence of another embodiment (right ITR sequence taken from a commercially available recombinant AAV genome plasmid) of the ITR is represented herein as SEQ ID No: 66, as follows:

[SEQ ID No: 66]
AGGAACCCCTAGTGATGGAGTTGGCCACTCCCTCTCTGC
GCGCTCGCTCGCTCACTGAGGCCGGGCGACCAAAGGTCG
CCCGACGCCCGGGCTTTGCCCGGGCGGCCTCAGTGAGCG
AGCGAGCGCGC

Preferably, the left and/or right Inverted Terminal Repeats comprise a nucleic acid sequence substantially as set out in SEQ ID No: 65 or 66, or a fragment or variant thereof.

Recently, it has been discovered that non-coding introns located between the promoter and the gene (next to the 3′ end of a promoter and the 5′ end of the gene) can facilitate gene expression in certain genomic sequences through mRNA accumulation [51,52]. Therefore, inclusion of an intron to the genetic construct of a viral vector may facilitate greater transgene expression and subsequent generation of mature proteins when used in combination with a constitutive or regulated promoter.

Hence, in one embodiment, the genetic construct comprises a non-coding intron. Preferably, the non-coding intron is located between the promoter and the first coding sequence. In other words, the non-coding intron is disposed 3′ of the promoter and 5′ of the first coding sequence.

In one embodiment, the non-coding intron is a minute virus of mice (MVM) small (121 bp) intron [53], referred to herein as SEQ ID No: 67, as follows:

[SEQ ID No: 67]
AGGTACGATGGCGCCTCCAGCTAAAAGAGCTAAAAGAGG
TAAGGGTTTAAGGGATGGTTGGTTGGTGGGGTATTAATG
TTTAATTACCTGTTTTACAGGCCTGAAATCACTTGGTTT
TAGG

In another embodiment, the non-coding intron is a sequence (133 bp) from the 5′-donor sites of the first intron of the human β-globin gene and the branch and 3′-acceptor sites from the intron of an immunoglobulin gene heavy chain variable region, referred to herein as SEQ ID No: 68, as follows:

[SEQ ID No: 68]
GTAAGTATCAAGGTTACAAGACAGGTTTAAGGAGACCAA
TAGAAACTGGGCTTGTCGAGACAGAGAAGACTCTTGCGT
TTCTGATAGGCACCTATTGGTCTTACTGACATCCACTTT
GCCTTTCTCTCCACAG

In another embodiment, the non-coding intron is a fusion (210 bp) of 5′ and 3′ nucleotide components of the splice acceptor of the rabbit β-globulin gene 1, referred to herein as SEQ ID No: 69, as follows:

[SEQ ID No: 69]
GTGAGCGGGCGGGACGGCCCTTCTCCTCCGGGCTGTAAT
TAGCGCTTGGTTTAATGACGGCTTGTTTCTTTTCTGTGG
CTGCGTGAAAGCCTTGAGGGGCTCCGGGAGGCTAGAGCC
TCTGCTAACCATGTTCATGCCTTCTTCTTTTTCCTACAG
CTCCTGGGCAACGTGCTGGTTATTGTGCTGTCTCATCAT
TTTGGCAAAGAATTC

Therefore, preferably the non-coding intron comprises a nucleic acid sequence substantially as set out in SEQ ID No: 67, 68 or 69, or a fragment or variant thereof.

In order to allow the correct folding of the polypeptides encoded by the genetic construct, intracellular trafficking and secretion of the anti-VEGF protein and the anti-fibrotic protein from the target cells, the coding sequence for these proteins is preceded by a novel N-terminal minimal signal peptide coding sequence derived from the sequences of known secreted human proteins. The secretory signal peptides are comprised of a methionine initiator amino acid, a series of 2 or more basic amino acids (arginine or lysine), followed by a series of hydrophobic amino acids (leucine, isoleucine, valine or phenylalanine) and finally a cutting sequence to allow cleavage of the signal peptide from the final mature secreted protein.

Accordingly, in one embodiment, the genetic construct comprises a signal peptide coding sequence. Advantageously, this novel signal peptide coding sequence generated by the inventors optimises intracellular cleavage and trafficking of the secreted proteins within the cell. Preferably, the genetic construct comprises a first signal peptide coding sequence disposed before the first coding sequence, and a second signal peptide coding sequence disposed before the second coding sequence. Preferably, the first and second signal peptide coding sequences are disposed 5′ of the first and second coding sequences, respectively.

In one embodiment, the signal peptide coding sequence encodes an amino acid sequence referred to herein as SEQ ID No: 70, or a fragment or variant thereof, as set out below:

[SEQ ID No: 70]
MRRFLTVISFLLYFGCAFA

Preferably, in this embodiment, the signal peptide coding sequence is derived from human trypsin, and preferably comprises a nucleotide sequence (57 bp) referred herein as SEQ ID No: 71, or a fragment or variant thereof, as set out below:

[SEQ ID No: 71]
ATGCGCCGCTTCCTGACCGTGATCAGCTTCCTGCTGTACTTCGGCTGCG
CCTTCGCC

In an alternative embodiment, the signal peptide coding sequence is modified to enhance secretion from target cells. In this embodiment, the signal peptide coding sequence encodes an amino acid sequence referred to herein as SEQ ID No: 72, or a fragment or variant thereof, as set out below:

[SEQ ID No: 72]
MRRLVLLLCIGALLGHSKA

Preferably, in this embodiment, the signal peptide coding sequence comprises a nucleotide sequence referred herein as SEQ ID No: 73, or a fragment or variant thereof, as set out below:

[SEQ ID No: 73]
ATGCGCCGCCTGGTGCTGCTGCTGTGCATCGGCGCCCTGCTGGGCCACA
GCAAGGCC

In another embodiment, the signal peptide coding sequence encodes an amino acid sequence referred to herein as SEQ ID No: 74, or a fragment or variant thereof, as set out below:

[SEQ ID No: 74]
MRRLLTFISILA

Preferably, in this embodiment, the signal peptide coding sequence comprises a nucleotide sequence referred herein as SEQ ID No: 75, or a fragment or variant thereof, as set out below:

[SEQ ID No: 75]
ATGAAGAAGCTGCTGATCCTGGCCCTGGTGGGCGCCGCCGTGGCC

In another embodiment, the signal peptide coding sequence encodes an amino acid sequence referred to herein as SEQ ID No: 76, or a fragment or variant thereof, as set out below:

[SEQ ID No: 76]
MRRLLTFISILALVGAFA

Preferably, in this embodiment, the signal peptide coding sequence comprises a nucleotide sequence referred herein as SEQ ID No: 77, or a fragment or variant thereof, as set out below:

[SEQ ID No: 77]
ATGCGCCGCCTGCTGACCTTCATCAGCATCCTGGCCCTGGTGGGCGCCT
TCGCC

In another embodiment, the signal peptide coding sequence encodes an amino acid sequence referred to herein as SEQ ID No: 78, or a fragment or variant thereof, as set out below:

[SEQ ID No: 78]
MRRLLTFISILALVGAAFA

Preferably, in this embodiment, the signal peptide coding sequence comprises a nucleotide sequence referred herein as SEQ ID No: 79, or a fragment or variant thereof, as set out below:

[SEQ ID No: 79]
ATGCGCCGCCTGCTGACCTTCATCAGCATCCTGGCCCTGGTGGGCGCCG
CCTTCGCC

Therefore, preferably, the signal peptide coding sequence comprises a nucleotide sequence substantially as set out in any one of SEQ ID No: 71, 73, 75, 77 or 79 or a fragment or variant thereof. Preferably, the signal peptide coding sequence encodes an amino acid sequence substantially as set out in SEQ ID No: 70, 72, 74, 76 or 78, or a fragment or variant thereof.

In a preferred embodiment, the genetic construct may comprise, in this specified order, a 5′ promoter; a first coding sequence encoding an anti-VEGF protein; and a 3′ second coding sequence encoding an anti-fibrotic protein. The use of 5′ and 3′ indicates that the features are either upstream or downstream, and is not intended to indicate that the features are necessarily terminal features. Furthermore, the skilled person would understand that the first and second coding sequences encoding an anti-VEGF protein and an anti-fibrotic protein may be disposed in any 5′ to 3′ order.

In a particular embodiment, the genetic construct may comprise in this specified order, a 5′ promoter; a first coding sequence encoding an anti-VEGF protein; a spacer sequence; and a 3′ second coding sequence encoding an anti-fibrotic protein.

In a particular embodiment, the genetic construct may comprise in this specified order, a 5′ promoter; a first coding sequence encoding an anti-VEGF protein; a viral-2A removal sequence; a spacer sequence; and a 3′ second coding sequence encoding an anti-fibrotic protein.

In a particular embodiment, the genetic construct may comprise in this specified order, a 5′ ITR; a promoter; a first coding sequence encoding an anti-VEGF protein; a viral-2A removal sequence; a spacer sequence; a second coding sequence encoding an anti-fibrotic protein; a sequence encoding WPRE; a sequence encoding a poly A tail; and a 3′ ITR.

In a particular embodiment, the genetic construct may comprise in this specified order, a 5′ ITR; a promoter; a non-coding intron; a first coding sequence encoding an anti-VEGF protein; a viral-2A removal sequence; a spacer sequence; a second coding sequence encoding an anti-fibrotic protein; a sequence encoding WPRE; a sequence encoding a poly A tail; and a 3′ ITR.

In a particular embodiment, the genetic construct may comprise in this specified order, a 5′ ITR; a promoter; a non-coding intron; a first signal peptide coding sequence; a first coding sequence encoding an anti-VEGF protein; a viral-2A removal sequence; a spacer sequence; a second signal peptide coding sequence; a second coding sequence encoding an anti-fibrotic protein; a sequence encoding WPRE; a sequence encoding a poly A tail; and a 3′ ITR.

From the foregoing, the skilled person will appreciate the nucleotide sequence of an embodiment of the construct of the first aspect, as well as the amino acid sequence of the encoded transgene. However, for the avoidance of doubt, in one embodiment, the amino acid sequence of [VEGF capture protein-2-furin-P2A-anti-CTGF SCVF-1], is referred to herein as SEQ ID No: 80, as follows:

[SEQ ID No: 80]
MRRLLTFISILALVGAAFASDTGRPFVEMYSEIPEIIHM
TEGRELVIPCRVTSPNITVILKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKFLSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLTV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRFLTVISFLLYFGCAFADIQMTQSPSS
LSASVGDRVTITCRASQGISSWLAWYQQKPEKAPKSLIY
AASSLQSGVPSRFSGSGSGTDFTLTISSLQPEDFATYYC
QQYNSYPPTFGQGTKLEIKRGGGGGSGGGGSGGGGSGGG
GSEGQLVQSGGGLVHPGGSLRLSCAGSGFTFSSYGMHWV
RQAPGKGLEWVSGIGTGGGTYSTDSVKGRFTISRDNAKN
SLYLQMNSLRAEDMAVYYCARGDYYGSGSFFDCWGQGTL
VTVSS

Preferably, in this embodiment, the construct comprises a 2241 nucleotide sequence (contained within the plasmid IKC153P), which is referred to herein as SEQ ID No: 81, or a fragment or variant thereof, as follows:

[SEQ ID No: 81]
ATGCGCCGCCTGCTGACCTTCATCAGCATCCTGGCCCTG
GTGGGCGCCGCCTTCGCCAGCGACACCGGCCGCCCCTTC
GTGGAGATGTACAGCGAGATCCCCGAGATCATCCACATG
ACCGAGGGCCGCGAGCTGGTGATCCCCTGCCGCGTGACC
AGCCCCAACATCACCGTGACCCTGAAGAAGTTCCCCCTG
GACACCCTGATCCCCGACGGCAAGCGCATCATCTGGGAC
AGCCGCAAGGGCTTCATCATCAGCAACGCCACCTACAAG
GAGATCGGCCTGCTGACCTGCGAGGCCACCGTGAACGGC
CACCTGTACAAGACCAACTACCTGACCCACCGCCAGACC
AACACCATCATCGACGTGGTGCTGAGCCCCAGCCACGGC
ATCGAGCTGAGCGTGGGCGAGAAGCTGGTGCTGAACTGC
ACCGCCCGCACCGAGCTGAACGTGGGCATCGACTTCAAC
TGGGAGTACCCCAGCAGCAAGCACCAGCACAAGAAGCTG
GTGAACCGCGACCTGAAGACCCAGAGCGGCAGCGAGATG
AAGAAGTTCCTGAGCACCCTGACCATCGACGGCGTGACC
CGCAGCGACCAGGGCCTGTACACCTGCGCCGCCAGCAGC
GGCCTGATGACCAAGAAGAACAGCACCTTCGTGCGCGTG
CACGAGAAGGACAAGACCCACACCTGCCCCCCCTGCCCC
GCCCCCGAGGCCGCCGGCGGCCCCAGCGTGTTCCTGTTC
CCCCCCAAGCCCAAGGACACCCTGATGATCAGCCGCACC
CCCGAGGTGACCTGCGTGGTGGTGGACGTGAGCCACGAG
GACCCCGAGGTGAAGTTCAACTGGTACGTGGACGGCGTG
GAGGTGCACAACGCCAAGACCAAGCCCCGCGAGGAGCAG
TACAACAGCACCTACCGCGTGGTGAGCGTGCTGACCGTG
CTGGCCCAGGACTGGCTGAACGGCAAGGAGTACAAGTGC
AAGGTGAGCAACAAGGCCCTGGGCGCCCCCATCGAGAAG
ACCATCAGCAAGGCCAAGGGCCAGCCCCGCGAGCCCCAG
GTGTACACCCTGCCCCCCAGCCGCGACGAGCTGACCAAG
AACCAGGTGAGCCTGACCTGCCTGGTGAAGGGCTTCTAC
CCCAGCGACATCGCCGTGGAGTGGGAGAGCAACGGCCAG
CCCGAGAACAACTACAAGACCACCCCCCCCGTGCTGGAC
AGCGACGGCAGCTTCTTCCTGTACAGCAAGCTGACCGTG
GACAAGAGCCGCTGGCAGCAGGGCAACGTGTTCAGCTGC
AGCGTGATGCACGAGGCCCTGCACAACGCCTACACCCAG
AAGAGCCTGAGCCTGAGCCCCGGCCGCCGCAGCAAGCGC
AGCGGCAGCGGCGCCACCAACTTCAGCCTGCTGAAGCAG
GCCGGCGACGTGGAGGAGAACCCCGGCCCCATGCGCCGC
TTCCTGACCGTGATCAGCTTCCTGCTGTACTTCGGCTGC
GCCTTCGCCGACATCCAGATGACCCAGAGCCCCAGCAGC
CTGAGCGCCAGCGTGGGCGACCGCGTGACCATCACCTGC
CGCGCCAGCCAGGGCATCAGCAGCTGGCTGGCCTGGTAC
CAGCAGAAGCCCGAGAAGGCCCCCAAGAGCCTGATCTAC
GCCGCCAGCAGCCTCCAGAGCGGCGTGCCCAGCCGCTTC
AGCGGCAGCGGCAGCGGCACCGACTTCACCCTGACCATC
AGCAGCCTCCAGCCCGAGGACTTCGCCACCTACTACTGC
CAGCAGTACAACAGCTACCCCCCCACCTTCGGCCAGGGC
ACCAAGCTGGAGATCAAGCGCGGCGGCGGCGGCGGCAGC
GGCGGCGGCGGCAGCGGCGGCGGCGGCAGCGGCGGCGGC
GGCAGCGAGGGCCAGCTGGTGCAGAGCGGCGGCGGCCTG
GTGCACCCCGGCGGCAGCCTGCGCCTGAGCTGCGCCGGC
AGCGGCTTCACCTTCAGCAGCTACGGCATGCACTGGGTG
CGCCAGGCCCCCGGCAAGGGCCTGGAGTGGGTGAGCGGC
ATCGGCACCGGCGGCGGCACCTACAGCACCGACAGCGTG
AAGGGCCGCTTCACCATCAGCCGCGACAACGCCAAGAAC
AGCCTGTACCTCCAGATGAACAGCCTGCGCGCCGAGGAC
ATGGCCGTGTACTACTGCGCCCGCGGCGACTACTACGGC
AGCGGCAGCTTCTTCGACTGCTGGGGCCAGGGCACCCTG
GTGACCGTGAGCAGCTAA

In another embodiment, the amino acid sequence of [VEGF capture protein-2-furin-P2A-anti-CTGF SCVF-2], is referred to herein as SEQ ID No: 82, or a fragment or variant thereof, as follows:

[SEQ ID No: 82]
MRRLLTFISILALVGAAFASDTGRPFVEMYSEIPEIIHM
TEGRELVIPCRVTSPNITVTLKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKELSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLTV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRFLTVISFLLYFGCAFAAEVQLVESGG
GVVRPGGSLRLSCAASGFTFDDYGMSWVRQAPGKGLEWV
SYISSSGSTIYYADSVKGRFTISRDNAKNSLYLQMNSLR
AEDTAVYYCARGITETWGQGTLVTVSSGGGGSGGGGSGG
SALQSVLTQPPSASGTPGQRVTISCSGSSSNIGSNTVNW
YQQLPGTAPKLLIYSNNQRPSGVPDRFSGSKSGTSASLA
ISGLQSEDEADYYCAAWDDSLGAVFGGGTKLTVLGA

Preferably, in this embodiment, the construct comprises a 2217 nucleotide sequence (as contained within the plasmid IKC154P), which is referred to herein as SEQ ID No: 83, or a fragment or variant thereof, as follows:

[SEQ ID No: 83]
ATGCGCCGCCTGCTGACCTTCATCAGCATCCTGGCCCTG
GTGGGCGCCGCCTTCGCCAGCGACACCGGCCGCCCCTTC
GTGGAGATGTACAGCGAGATCCCCGAGATCATCCACATG
ACCGAGGGCCGCGAGCTGGTGATCCCCTGCCGCGTGACC
AGCCCCAACATCACCGTGACCCTGAAGAAGTTCCCCCTG
GACACCCTGATCCCCGACGGCAAGCGCATCATCTGGGAC
AGCCGCAAGGGCTTCATCATCAGCAACGCCACCTACAAG
GAGATCGGCCTGCTGACCTGCGAGGCCACCGTGAACGGC
CACCTGTACAAGACCAACTACCTGACCCACCGCCAGACC
AACACCATCATCGACGTGGTGCTGAGCCCCAGCCACGGC
ATCGAGCTGAGCGTGGGCGAGAAGCTGGTGCTGAACTGC
ACCGCCCGCACCGAGCTGAACGTGGGCATCGACTTCAAC
TGGGAGTACCCCAGCAGCAAGCACCAGCACAAGAAGCTG
GTGAACCGCGACCTGAAGACCCAGAGCGGCAGCGAGATG
AAGAAGTTCCTGAGCACCCTGACCATCGACGGCGTGACC
CGCAGCGACCAGGGCCTGTACACCTGCGCCGCCAGCAGC
GGCCTGATGACCAAGAAGAACAGCACCTTCGTGCGCGTG
CACGAGAAGGACAAGACCCACACCTGCCCCCCCTGCCCC
GCCCCCGAGGCCGCCGGCGGCCCCAGCGTGTTCCTGTTC
CCCCCCAAGCCCAAGGACACCCTGATGATCAGCCGCACC
CCCGAGGTGACCTGCGTGGTGGTGGACGTGAGCCACGAG
GACCCCGAGGTGAAGTTCAACTGGTACGTGGACGGCGTG
GAGGTGCACAACGCCAAGACCAAGCCCCGCGAGGAGCAG
TACAACAGCACCTACCGCGTGGTGAGCGTGCTGACCGTG
CTGGCCCAGGACTGGCTGAACGGCAAGGAGTACAAGTGC
AAGGTGAGCAACAAGGCCCTGGGCGCCCCCATCGAGAAG
ACCATCAGCAAGGCCAAGGGCCAGCCCCGCGAGCCCCAG
GTGTACACCCTGCCCCCCAGCCGCGACGAGCTGACCAAG
AACCAGGTGAGCCTGACCTGCCTGGTGAAGGGCTTCTAC
CCCAGCGACATCGCCGTGGAGTGGGAGAGCAACGGCCAG
CCCGAGAACAACTACAAGACCACCCCCCCCGTGCTGGAC
AGCGACGGCAGCTTCTTCCTGTACAGCAAGCTGACCGTG
GACAAGAGCCGCTGGCAGCAGGGCAACGTGTTCAGCTGC
AGCGTGATGCACGAGGCCCTGCACAACGCCTACACCCAG
AAGAGCCTGAGCCTGAGCCCCGGCCGCCGCAGCAAGCGC
AGCGGCAGCGGCGCCACCAACTTCAGCCTGCTGAAGCAG
GCCGGCGACGTGGAGGAGAACCCCGGCCCCATGCGCCGC
TTCCTGACCGTGATCAGCTTCCTGCTGTACTTCGGCTGC
GCCTTCGCCGCCGAGGTGCAGCTGGTGGAGAGCGGCGGC
GGCGTGGTGCGGCCTGGCGGCAGCCTGCGCCTGAGCTGC
GCCGCCAGCGGCTTCACCTTCGACGACTACGGCATGAGC
TGGGTGCGCCAGGCTCCAGGCAAGGGCCTGGAGTGGGTG
AGCTACATCAGCAGCAGCGGCAGCACCATCTACTACGCC
GACAGCGTGAAGGGCCGCTTCACCATCAGCCGCGACAAC
GCCAAGAACAGCCTGTACCTGCAGATGAACAGCCTGCGC
GCCGAGGACACCGCCGTGTACTACTGCGCACGAGGCATC
ACCGAGACCTGGGGCCAGGGCACCCTGGTGACCGTGAGC
AGCGGCGGCGGCGGCAGCGGCGGCGGCGGCAGCGGCGGC
AGCGCCCTGCAGAGCGTGCTGACCCAGCCACCCAGCGCC
AGCGGCACCCCTGGCCAGCGCGTGACCATCAGCTGCAGC
GGCAGCAGCAGCAACATCGGCAGCAACACCGTGAACTGG
TACCAGCAGCTGCCCGGCACCGCGCCTAAGCTGCTGATC
TACAGCAACAACCAGCGCCCCAGCGGCGTGCCCGACCGC
TTCAGCGGCAGCAAGAGCGGCACCAGCGCCAGCCTGGCC
ATCAGCGGCCTGCAGAGCGAGGACGAGGCCGACTACTAC
TGCGCCGCCTGGGACGACAGCCTGGGCGCCGTGTTCGGC
GGCGGCACCAAGCTGACCGTGCTGGGCGCCTAA

In another embodiment, the amino acid sequence of [VEGF capture protein 2-furin-P2A-anti-C3b SCVF] is referred to herein as SEQ ID No: 84, or a fragment or variant thereof, as follows:

[SEQ ID No: 84]
MRRLLTFISILALVGAAFASDTGRPFVEMYSEIPEIIHM
TEGRELVIPCRVTSPNITVTLKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKFLSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLTV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRFLTVISFLLYFGCAFADIQMTQSPSS
LSASVGDRVTITCRASQDVSTAVAWYQQKPGKAPKLLIY
SASFLYSGVPSRFSGSGSGTDFTLTISSLQPEDFATYYC
QQSYATLPTFEQGTKVEIKRGGGGGSGGGGSGGGGSGGG
GSEVQLVESGGGLVQPGGSLRLSCAASGFSFTSSSVSPG
KGLEWVGLIYPYNGFNYYADSVKGRFTISADTSLQMNSL
RAEDTAVYYCARNALYGSGGYYAMDYWGQGTLVTVSS

Preferably, in this embodiment, the construct comprises a 2220 nucleotide sequence (as contained within the plasmid IKC129P), which is referred to herein as SEQ ID No: 85, or a fragment or variant thereof, as follows:

[SEQ ID No: 85]
ATGCGCCGCCTGCTGACCTTCATCAGCATCCTGGCCCTG
GTGGGCGCCGCCTTCGCCAGCGACACCGGCCGCCCCTTC
GTGGAGATGTACAGCGAGATCCCCGAGATCATCCACATG
ACCGAGGGCCGCGAGCTGGTGATCCCCTGCCGCGTGACC
AGCCCCAACATCACCGTGACCCTGAAGAAGTTCCCCCTG
GACACCCTGATCCCCGACGGCAAGCGCATCATCTGGGAC
AGCCGCAAGGGCTTCATCATCAGCAACGCCACCTACAAG
GAGATCGGCCTGCTGACCTGCGAGGCCACCGTGAACGGC
CACCTGTACAAGACCAACTACCTGACCCACCGCCAGACC
AACACCATCATCGACGTGGTGCTGAGCCCCAGCCACGGC
ATCGAGCTGAGCGTGGGCGAGAAGCTGGTGCTGAACTGC
ACCGCCCGCACCGAGCTGAACGTGGGCATCGACTTCAAC
TGGGAGTACCCCAGCAGCAAGCACCAGCACAAGAAGCTG
GTGAACCGCGACCTGAAGACCCAGAGCGGCAGCGAGATG
AAGAAGTTCCTGAGCACCCTGACCATCGACGGCGTGACC
CGCAGCGACCAGGGCCTGTACACCTGCGCCGCCAGCAGC
GGCCTGATGACCAAGAAGAACAGCACCTTCGTGCGCGTG
CACGAGAAGGACAAGACCCACACCTGCCCCCCCTGCCCC
GCCCCCGAGGCCGCCGGCGGCCCCAGCGTGTTCCTGTTC
CCCCCCAAGCCCAAGGACACCCTGATGATCAGCCGCACC
CCCGAGGTGACCTGCGTGGTGGTGGACGTGAGCCACGAG
GACCCCGAGGTGAAGTTCAACTGGTACGTGGACGGCGTG
GAGGTGCACAACGCCAAGACCAAGCCCCGCGAGGAGCAG
TACAACAGCACCTACCGCGTGGTGAGCGTGCTGACCGTG
CTGGCCCAGGACTGGCTGAACGGCAAGGAGTACAAGTGC
AAGGTGAGCAACAAGGCCCTGGGCGCCCCCATCGAGAAG
ACCATCAGCAAGGCCAAGGGCCAGCCCCGCGAGCCCCAG
GTGTACACCCTGCCCCCCAGCCGCGACGAGCTGACCAAG
AACCAGGTGAGCCTGACCTGCCTGGTGAAGGGCTTCTAC
CCCAGCGACATCGCCGTGGAGTGGGAGAGCAACGGCCAG
CCCGAGAACAACTACAAGACCACCCCCCCCGTGCTGGAC
AGCGACGGCAGCTTCTTCCTGTACAGCAAGCTGACCGTG
GACAAGAGCCGCTGGCAGCAGGGCAACGTGTTCAGCTGC
AGCGTGATGCACGAGGCCCTGCACAACGCCTACACCCAG
AAGAGCCTGAGCCTGAGCCCCGGCCGCCGCAGCAAGCGC
AGCGGCAGCGGCGCCACCAACTTCAGCCTGCTGAAGCAG
GCCGGCGACGTGGAGGAGAACCCCGGCCCCATGCGCCGC
TTCCTGACCGTGATCAGCTTCCTGCTGTACTTCGGCTGC
GCCTTCGCCGACATCCAGATGACCCAGAGCCCCAGCAGC
CTGAGCGCCAGCGTGGGCGACCGCGTGACCATCACCTGC
CGCGCCAGCCAGGACGTAAGCACCGCCGTGGCCTGGTAC
CAGCAGAAGCCCGGCAAGGCCCCCAAGCTGCTGATCTAC
AGCGCCAGCTTCCTGTACAGCGGCGTGCCCAGCCGCTTC
AGCGGCAGCGGCAGCGGCACCGACTTCACCCTGACCATC
AGCAGCCTGCAGCCCGAGGACTTCGCCACCTACTACTGC
CAGCAGAGCTACGCCACCCTGCCCACCTTCGAGCAGGGC
ACCAAGGTGGAGATCAAGCGCGGCGGCGGCGGCGGCAGC
GGCGGCGGCGGCAGCGGCGGCGGCGGCAGCGGCGGCGGC
GGCAGCGAGGTGCAGCTGGTGGAGAGCGGCGGCGGCCTG
GTGCAGCCCGGCGGCAGCCTGCGCCTGAGCTGCGCCGCC
AGCGGCTTCAGCTTCACCAGCAGCAGCGTGAGCCCCGGC
AAGGGCCTGGAGTGGGTGGGCCTGATCTACCCCTACAAC
GGCTTCAACTACTACGCCGACAGCGTGAAGGGCCGCTTC
ACCATCAGCGCCGACACCAGCCTGCAGATGAACAGCCTG
CGCGCCGAGGACACCGCCGTGTACTACTGCGCCCGCAAC
GCCCTGTACGGCAGCGGCGGCTACTACGCCATGGACTAC
TGGGGCCAGGGCACCCTGGTGACCGTGAGCAGCTGA

In another embodiment, the amino acid sequence of [VEGF capture protein 2-furin-P2A-anti-Bb SCVF] is referred to herein as SEQ ID No: 86, or a fragment or variant thereof, as follows:

[SEQ ID No: 86]
MRRLLTFISILALVGAAFASDTGRPFVEMYSEIPEIIHM
TEGRELVIPCRVTSPNITVILKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKFLSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLTV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRFLTVISFLLYFGCAFADVQITQSPSY
LAASPGETITINCRASKSISKYLAWYQDKPGKINKLLIY
SGSTLQSGIPSRFSGSGSGTDFTLTISSLEPEDFAMYYC
QQHDEYPWTFGGGTKLEIKRGGGGGSGGGGSGGGGSGGG
GSQVQLQQSGAELAKPGASVRMSCKASGYTFTNYWIHWV
KQRPGQGLEWIGYINPNTGYNDYNQKFKDKATLTADKSS
STVYMQLSSLTSEDSAVYYCARGGQLGLRRAMDYWGQGT
SVTVSS

Preferably, in this embodiment, the construct comprises a 2244 nucleotide sequence (as contained within the plasmid IKC130P), which is referred to herein as SEQ ID No: 87, or a fragment or variant thereof, as follows:

[SEQ ID No: 87]
ATGCGCCGCCTGCTGACCTTCATCAGCATCCTGGCCCTG
GTGGGCGCCGCCTTCGCCAGCGACACCGGCCGCCCCTTC
GTGGAGATGTACAGCGAGATCCCCGAGATCATCCACATG
ACCGAGGGCCGCGAGCTGGTGATCCCCTGCCGCGTGACC
AGCCCCAACATCACCGTGACCCTGAAGAAGTTCCCCCTG
GACACCCTGATCCCCGACGGCAAGCGCATCATCTGGGAC
AGCCGCAAGGGCTTCATCATCAGCAACGCCACCTACAAG
GAGATCGGCCTGCTGACCTGCGAGGCCACCGTGAACGGC
CACCTGTACAAGACCAACTACCTGACCCACCGCCAGACC
AACACCATCATCGACGTGGTGCTGAGCCCCAGCCACGGC
ATCGAGCTGAGCGTGGGCGAGAAGCTGGTGCTGAACTGC
ACCGCCCGCACCGAGCTGAACGTGGGCATCGACTTCAAC
TGGGAGTACCCCAGCAGCAAGCACCAGCACAAGAAGCTG
GTGAACCGCGACCTGAAGACCCAGAGCGGCAGCGAGATG
AAGAAGTTCCTGAGCACCCTGACCATCGACGGCGTGACC
CGCAGCGACCAGGGCCTGTACACCTGCGCCGCCAGCAGC
GGCCTGATGACCAAGAAGAACAGCACCTTCGTGCGCGTG
CACGAGAAGGACAAGACCCACACCTGCCCCCCCTGCCCC
GCCCCCGAGGCCGCCGGCGGCCCCAGCGTGTTCCTGTTC
CCCCCCAAGCCCAAGGACACCCTGATGATCAGCCGCACC
CCCGAGGTGACCTGCGTGGTGGTGGACGTGAGCCACGAG
GACCCCGAGGTGAAGTTCAACTGGTACGTGGACGGCGTG
GAGGTGCACAACGCCAAGACCAAGCCCCGCGAGGAGCAG
TACAACAGCACCTACCGCGTGGTGAGCGTGCTGACCGTG
CTGGCCCAGGACTGGCTGAACGGCAAGGAGTACAAGTGC
AAGGTGAGCAACAAGGCCCTGGGCGCCCCCATCGAGAAG
ACCATCAGCAAGGCCAAGGGCCAGCCCCGCGAGCCCCAG
GTGTACACCCTGCCCCCCAGCCGCGACGAGCTGACCAAG
AACCAGGTGAGCCTGACCTGCCTGGTGAAGGGCTTCTAC
CCCAGCGACATCGCCGTGGAGTGGGAGAGCAACGGCCAG
CCCGAGAACAACTACAAGACCACCCCCCCCGTGCTGGAC
AGCGACGGCAGCTTCTTCCTGTACAGCAAGCTGACCGTG
GACAAGAGCCGCTGGCAGCAGGGCAACGTGTTCAGCTGC
AGCGTGATGCACGAGGCCCTGCACAACGCCTACACCCAG
AAGAGCCTGAGCCTGAGCCCCGGCCGCCGCAGCAAGCGC
AGCGGCAGCGGCGCCACCAACTTCAGCCTGCTGAAGCAG
GCCGGCGACGTGGAGGAGAACCCCGGCCCCATGCGCCGC
TTCCTGACCGTGATCAGCTTCCTGCTGTACTTCGGCTGC
GCCTTCGCCGACGTGCAGATCACCCAGAGCCCCAGCTAC
CTGGCCGCCAGCCCCGGCGAGACCATCACCATCAACTGC
CGCGCCAGCAAGAGCATCAGCAAGTACCTGGCCTGGTAC
CAGGACAAGCCCGGCAAGACCAACAAGCTGCTGATCTAC
AGCGGCAGCACCCTGCAGAGCGGCATCCCCAGCCGCTTC
AGCGGCAGCGGCAGCGGCACCGACTTCACCCTGACCATC
AGCAGCCTGGAGCCCGAGGACTTCGCCATGTACTACTGC
CAGCAGCACGACGAGTACCCCTGGACCTTCGGCGGCGGC
ACCAAGCTGGAGATCAAGCGCGGCGGCGGCGGCGGCAGC
GGCGGCGGCGGCAGCGGCGGCGGCGGCAGCGGCGGCGGC
GGCAGCCAGGTGCAGCTGCAGCAGAGCGGCGCCGAGCTG
GCCAAGCCCGGCGCCAGCGTGCGCATGAGCTGCAAGGCC
AGCGGCTACACCTTCACCAACTACTGGATCCACTGGGTG
AAGCAGCGCCCCGGCCAGGGCCTGGAGTGGATCGGCTAC
ATCAACCCCAACACCGGCTACAACGACTACAACCAGAAG
TTCAAGGACAAGGCCACCCTGACCGCCGACAAGAGCAGC
AGCACCGTGTACATGCAGCTGAGCAGCCTGACCAGCGAG
GACAGCGCCGTGTACTACTGCGCCCGCGGCGGCCAGCTG
GGCCTGCGCCGCGCCATGGACTACTGGGGCCAGGGCACC
AGCGTGACCGTGAGCAGCTGA

In another embodiment, the amino acid sequence of [VEGF capture protein 2-furin-P2A-sCD55, is referred to herein as SEQ ID No: 88 or a fragment or variant thereof, as follows:

[SEQ ID No: 88]
MRRLLTFISILALVGAAFASDTGRPFVEMYSEIPEIIHM
TEGRELVIPCRVTSPNITVTLKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKFLSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLTV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRFLTVISFLLYFGCAFADCGLPPDVPN
AQPALEGRTSFPEDTVITYKCEESFVKIPGEKDSVICLK
GSQWSDIEEFCNRSCEVPTRLNSASLKQPYITQNYFPVG
TVVEYECRPGYRREPSLSPKLTCLQNLKWSTAVEFCKKK
SCPNPGEIRNGQIDVPGGILFGATISFSCNTGYKLFGST
SSFCLISGSSVQWSDPLPECREIYCPAPPQIDNGIIQGE
RDHYGYRQSVTYACNKGFTMIGEHSIYCTVNNDEGEWSG
PPPECRGKSLTSKVPPTVQKPTTVNVPTTEVSPTSQKTT
TKTTTPNAQATRSTPVSRTTKHFHETTPNKGSGTTSG

Preferably, in this embodiment, the construct comprises a 2454 nucleotide sequence (as contained within the plasmid IKC131P), which is referred to herein as SEQ ID No: 89, or a fragment or variant thereof, as follows:

[SEQ ID No: 89]
ATGCGCCGCCTGCTGACCTTCATCAGCATCCTGGCCCTG
GTGGGCGCCGCCTTCGCCAGCGACACCGGCCGCCCCTTC
GTGGAGATGTACAGCGAGATCCCCGAGATCATCCACATG
ACCGAGGGCCGCGAGCTGGTGATCCCCTGCCGCGTGACC
AGCCCCAACATCACCGTGACCCTGAAGAAGTTCCCCCTG
GACACCCTGATCCCCGACGGCAAGCGCATCATCTGGGAC
AGCCGCAAGGGCTTCATCATCAGCAACGCCACCTACAAG
GAGATCGGCCTGCTGACCTGCGAGGCCACCGTGAACGGC
CACCTGTACAAGACCAACTACCTGACCCACCGCCAGACC
AACACCATCATCGACGTGGTGCTGAGCCCCAGCCACGGC
ATCGAGCTGAGCGTGGGCGAGAAGCTGGTGCTGAACTGC
ACCGCCCGCACCGAGCTGAACGTGGGCATCGACTTCAAC
TGGGAGTACCCCAGCAGCAAGCACCAGCACAAGAAGCTG
GTGAACCGCGACCTGAAGACCCAGAGCGGCAGCGAGATG
AAGAAGTTCCTGAGCACCCTGACCATCGACGGCGTGACC
CGCAGCGACCAGGGCCTGTACACCTGCGCCGCCAGCAGC
GGCCTGATGACCAAGAAGAACAGCACCTTCGTGCGCGTG
CACGAGAAGGACAAGACCCACACCTGCCCCCCCTGCCCC
GCCCCCGAGGCCGCCGGCGGCCCCAGCGTGTTCCTGTTC
CCCCCCAAGCCCAAGGACACCCTGATGATCAGCCGCACC
CCCGAGGTGACCTGCGTGGTGGTGGACGTGAGCCACGAG
GACCCCGAGGTGAAGTTCAACTGGTACGTGGACGGCGTG
GAGGTGCACAACGCCAAGACCAAGCCCCGCGAGGAGCAG
TACAACAGCACCTACCGCGTGGTGAGCGTGCTGACCGTG
CTGGCCCAGGACTGGCTGAACGGCAAGGAGTACAAGTGC
AAGGTGAGCAACAAGGCCCTGGGCGCCCCCATCGAGAAG
ACCATCAGCAAGGCCAAGGGCCAGCCCCGCGAGCCCCAG
GTGTACACCCTGCCCCCCAGCCGCGACGAGCTGACCAAG
AACCAGGTGAGCCTGACCTGCCTGGTGAAGGGCTTCTAC
CCCAGCGACATCGCCGTGGAGTGGGAGAGCAACGGCCAG
CCCGAGAACAACTACAAGACCACCCCCCCCGTGCTGGAC
AGCGACGGCAGCTTCTTCCTGTACAGCAAGCTGACCGTG
GACAAGAGCCGCTGGCAGCAGGGCAACGTGTTCAGCTGC
AGCGTGATGCACGAGGCCCTGCACAACGCCTACACCCAG
AAGAGCCTGAGCCTGAGCCCCGGCCGCCGCAGCAAGCGC
AGCGGCAGCGGCGCCACCAACTTCAGCCTGCTGAAGCAG
GCCGGCGACGTGGAGGAGAACCCCGGCCCCATGCGCCGC
TTCCTGACCGTGATCAGCTTCCTGCTGTACTTCGGCTGC
GCCTTCGCCGACTGCGGCCTGCCCCCCGACGTGCCCAAC
GCCCAGCCCGCCCTGGAGGGCCGCACCAGCTTCCCCGAG
GACACCGTGATCACCTACAAGTGCGAGGAGAGCTTCGTG
AAGATCCCCGGCGAGAAGGACAGCGTGATCTGCCTGAAG
GGCAGCCAGTGGAGCGACATCGAGGAGTTCTGCAACCGC
AGCTGCGAGGTGCCCACCCGCCTGAACAGCGCCAGCCTG
AAGCAGCCCTACATCACCCAGAACTACTTCCCCGTGGGC
ACCGTGGTGGAGTACGAGTGCCGCCCCGGCTACCGCCGC
GAGCCCAGCCTGAGCCCCAAGCTGACCTGCCTGCAGAAC
CTGAAGTGGAGCACCGCCGTGGAGTTCTGCAAGAAGAAG
AGCTGCCCCAACCCCGGCGAGATCCGCAACGGCCAGATC
GACGTGCCCGGCGGCATCCTGTTCGGCGCCACCATCAGC
TTCAGCTGCAACACCGGCTACAAGCTGTTCGGCAGCACC
AGCAGCTTCTGCCTGATCAGCGGCAGCAGCGTGCAGTGG
AGCGACCCCCTGCCCGAGTGCCGCGAGATCTACTGCCCC
GCCCCCCCCCAGATCGACAACGGCATCATCCAGGGCGAG
CGCGACCACTACGGCTACCGCCAGAGCGTGACCTACGCC
TGCAACAAGGGCTTCACCATGATCGGCGAGCACAGCATC
TACTGCACCGTGAACAACGACGAGGGCGAGTGGAGCGGC
CCCCCCCCCGAGTGCCGAGGCAAGAGCCTGACCAGCAAG
GTGCCCCCCACCGTGCAGAAGCCCACCACCGTGAACGTG
CCCACCACCGAGGTGAGCCCCACCAGCCAGAAGACCACC
ACCAAGACCACCACCCCCAACGCCCAGGCCACCCGCAGC
ACCCCCGTGAGCCGCACCACCAAGCACTTCCACGAGACC
ACCCCCAACAAGGGCAGCGGCACCACCAGCGGCTAA

In another embodiment, the amino acid sequence of [sCD55-furin-P2A-VEGF capture protein 2], is referred to herein as SEQ ID No: 90 or a fragment or variant thereof, as follows:

[SEQ ID No: 90]
MRRFLTVISFLLYFGCAFADCGLPPDVPNAQPALEGRTS
FPEDTVITYKCEESFVKIPGEKDSVICLKGSQWSDIEEF
CNRSCEVPTRLNSASLKQPYITQNYFPVGTVVEYECRPG
YRREPSLSPKLTCLQNLKWSTAVEFCKKKSCPNPGEIRN
GQIDVPGGILFGATISFSCNTGYKLFGSTSSFCLISGSS
VQWSDPLPECREIYCPAPPQIDNGIIQGERDHYGYRQSV
TYACNKGFTMIGEHSIYCTVNNDEGEWSGPPPECRGKSL
TSKVPPTVQKPTTVNVPTTEVSPTSQKTTTKTTTPNAQA
TRSTPVSRTTKHFHETTPNKGSGTTSGRRSKRSGSGATN
FSLLKQAGDVEENPGPMRRLLTFISILALVGAAFASDTG
RPFVEMYSEIPEIIHMTEGRELVIPCRVTSPNITVILKK
FPLDTLIPDGKRIIWDSRKGFIISNATYKEIGLLTCEAT
VNGHLYKTNYLTHRQTNTIIDVVLSPSHGIELSVGEKLV
LNCTARTELNVGIDENWEYPSSKHQHKKLVNRDLKTQSG
SEMKKFLSTLTIDGVTRSDQGLYTCAASSGLMTKKNSTF
VRVHEKDKTHTCPPCPAPEAAGGPSVFLFPPKPKDTLMI
SRTPEVTCVVVDVSHEDPEVKFNWYVDGVEVHNAKTKPR
EEQYNSTYRVVSVLTVLAQDWLNGKEYKCKVSNKALGAP
IEKTISKAKGQPREPQVYTLPPSRDELTKNQVSLTCLVK
GFYPSDIAVEWESNGQPENNYKTTPPVLDSDGSFFLYSK
LTVDKSRWQQGNVFSCSVMHEALHNAYTQKSLSLSPG

Preferably, in this embodiment, the construct comprises a 2454 nucleotide sequence (as contained within the plasmid IKC132P), which is referred to herein as SEQ ID No: 91, or a fragment or variant thereof, as follows:

[SEQ ID No: 91]
ATGCGCCGCTTCCTGACCGTGATCAGCTTCCTGCTGTAC
TTCGGCTGCGCCTTCGCCGACTGCGGCCTGCCCCCCGAC
GTGCCCAACGCCCAGCCCGCCCTGGAGGGCCGCACCAGC
TTCCCCGAGGACACCGTGATCACCTACAAGTGCGAGGAG
AGCTTCGTGAAGATCCCCGGCGAGAAGGACAGCGTGATC
TGCCTGAAGGGCAGCCAGTGGAGCGACATCGAGGAGTTC
TGCAACCGCAGCTGCGAGGTGCCCACCCGCCTGAACAGC
GCCAGCCTGAAGCAGCCCTACATCACCCAGAACTACTTC
CCCGTGGGCACCGTGGTGGAGTACGAGTGCCGCCCCGGC
TACCGCCGCGAGCCCAGCCTGAGCCCCAAGCTGACCTGC
CTGCAGAACCTGAAGTGGAGCACCGCCGTGGAGTTCTGC
AAGAAGAAGAGCTGCCCCAACCCCGGCGAGATCCGCAAC
GGCCAGATCGACGTGCCCGGCGGCATCCTGTTCGGCGCC
ACCATCAGCTTCAGCTGCAACACCGGCTACAAGCTGTTC
GGCAGCACCAGCAGCTTCTGCCTGATCAGCGGCAGCAGC
GTGCAGTGGAGCGACCCCCTGCCCGAGTGCCGCGAGATC
TACTGCCCCGCCCCCCCCCAGATCGACAACGGCATCATC
CAGGGCGAGCGCGACCACTACGGCTACCGCCAGAGCGTG
ACCTACGCCTGCAACAAGGGCTTCACCATGATCGGCGAG
CACAGCATCTACTGCACCGTGAACAACGACGAGGGCGAG
TGGAGCGGCCCCCCCCCCGAGTGCCGAGGCAAGAGCCTG
ACCAGCAAGGTGCCCCCCACCGTGCAGAAGCCCACCACC
GTGAACGTGCCCACCACCGAGGTGAGCCCCACCAGCCAG
AAGACCACCACCAAGACCACCACCCCCAACGCCCAGGCC
ACCCGCAGCACCCCCGTGAGCCGCACCACCAAGCACTTC
CACGAGACCACCCCCAACAAGGGCAGCGGCACCACCAGC
GGCCGCCGCAGCAAGCGCAGCGGCAGCGGCGCCACCAAC
TTCAGCCTGCTGAAGCAGGCCGGCGACGTGGAGGAGAAC
CCCGGCCCCATGCGCCGCCTGCTGACCTTCATCAGCATC
CTGGCCCTGGTGGGCGCCGCCTTCGCCAGCGACACCGGC
CGCCCCTTCGTGGAGATGTACAGCGAGATCCCCGAGATC
ATCCACATGACCGAGGGCCGCGAGCTGGTGATCCCCTGC
CGCGTGACCAGCCCCAACATCACCGTGACCCTGAAGAAG
TTCCCCCTGGACACCCTGATCCCCGACGGCAAGCGCATC
ATCTGGGACAGCCGCAAGGGCTTCATCATCAGCAACGCC
ACCTACAAGGAGATCGGCCTGCTGACCTGCGAGGCCACC
GTGAACGGCCACCTGTACAAGACCAACTACCTGACCCAC
CGCCAGACCAACACCATCATCGACGTGGTGCTGAGCCCC
AGCCACGGCATCGAGCTGAGCGTGGGCGAGAAGCTGGTG
CTGAACTGCACCGCCCGCACCGAGCTGAACGTGGGCATC
GACTTCAACTGGGAGTACCCCAGCAGCAAGCACCAGCAC
AAGAAGCTGGTGAACCGCGACCTGAAGACCCAGAGCGGC
AGCGAGATGAAGAAGTTCCTGAGCACCCTGACCATCGAC
GGCGTGACCCGCAGCGACCAGGGCCTGTACACCTGCGCC
GCCAGCAGCGGCCTGATGACCAAGAAGAACAGCACCTTC
GTGCGCGTGCACGAGAAGGACAAGACCCACACCTGCCCC
CCCTGCCCCGCCCCCGAGGCCGCCGGCGGCCCCAGCGTG
TTCCTGTTCCCCCCCAAGCCCAAGGACACCCTGATGATC
AGCCGCACCCCCGAGGTGACCTGCGTGGTGGTGGACGTG
AGCCACGAGGACCCCGAGGTGAAGTTCAACTGGTACGTG
GACGGCGTGGAGGTGCACAACGCCAAGACCAAGCCCCGC
GAGGAGCAGTACAACAGCACCTACCGCGTGGTGAGCGTG
CTGACCGTGCTGGCCCAGGACTGGCTGAACGGCAAGGAG
TACAAGTGCAAGGTGAGCAACAAGGCCCTGGGCGCCCCC
ATCGAGAAGACCATCAGCAAGGCCAAGGGCCAGCCCCGC
GAGCCCCAGGTGTACACCCTGCCCCCCAGCCGCGACGAG
CTGACCAAGAACCAGGTGAGCCTGACCTGCCTGGTGAAG
GGCTTCTACCCCAGCGACATCGCCGTGGAGTGGGAGAGC
AACGGCCAGCCCGAGAACAACTACAAGACCACCCCCCCC
GTGCTGGACAGCGACGGCAGCTTCTTCCTGTACAGCAAG
CTGACCGTGGACAAGAGCCGCTGGCAGCAGGGCAACGTG
TTCAGCTGCAGCGTGATGCACGAGGCCCTGCACAACGCC
TACACCCAGAAGAGCCTGAGCCTGAGCCCCGGCTAA

In another embodiment, the amino acid sequence of [VEGF capture protein 2-furin-P2A-CFHR1], is referred to herein as SEQ ID No: 92, or a fragment or variant thereof, as follows:

[SEQ ID No: 92]
MRRLLTFISILALVGAAFASDTGRPFVEMYSEIPEIIHM
TEGRELVIPCRVTSPNITVTLKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKFLSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLTV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRFLTVISFLLYFGCAFAEATFCDFPKI
NHGILYDEEKYKPFSQVPTGEVFYYSCEYNFVSPSKSFW
TRITCTEEGWSPTPKCLRLCFFPFVENGHSESSGQTHLE
GDTVQIICNTGYRLQNNENNISCVERGWSTPPKCRSTDT
SCVNPPTVQNAHILSRQMSKYPSGERVRYECRSPYEMFG
DEEVMCLNGNWTEPPQCKDSTGKCGPPPPIDNGDITSFP
LSVYAPASSVEYQCQNLYQLEGNKRITCRNGQWSEPPKC
LHPCVISREIMENYNIALRWTAKQKLYLRTGESAEFVCK
RGYRLSSRSHTLRTTCWDGKLEYPTCAKR

Preferably, in this embodiment, the construct comprises a 2430 nucleotide sequence (as contained within the plasmid IKC133P), which is referred to herein as SEQ ID No: 93, or a fragment or variant thereof, as follows:

[SEQ ID No: 93]
ATGCGCCGCCTGCTGACCTTCATCAGCATCCTGGCCCTG
GTGGGCGCCGCCTTCGCCAGCGACACCGGCCGCCCCTTC
GTGGAGATGTACAGCGAGATCCCCGAGATCATCCACATG
ACCGAGGGCCGCGAGCTGGTGATCCCCTGCCGCGTGACC
AGCCCCAACATCACCGTGACCCTGAAGAAGTTCCCCCTG
GACACCCTGATCCCCGACGGCAAGCGCATCATCTGGGAC
AGCCGCAAGGGCTTCATCATCAGCAACGCCACCTACAAG
GAGATCGGCCTGCTGACCTGCGAGGCCACCGTGAACGGC
CACCTGTACAAGACCAACTACCTGACCCACCGCCAGACC
AACACCATCATCGACGTGGTGCTGAGCCCCAGCCACGGC
ATCGAGCTGAGCGTGGGCGAGAAGCTGGTGCTGAACTGC
ACCGCCCGCACCGAGCTGAACGTGGGCATCGACTTCAAC
TGGGAGTACCCCAGCAGCAAGCACCAGCACAAGAAGCTG
GTGAACCGCGACCTGAAGACCCAGAGCGGCAGCGAGATG
AAGAAGTTCCTGAGCACCCTGACCATCGACGGCGTGACC
CGCAGCGACCAGGGCCTGTACACCTGCGCCGCCAGCAGC
GGCCTGATGACCAAGAAGAACAGCACCTTCGTGCGCGTG
CACGAGAAGGACAAGACCCACACCTGCCCCCCCTGCCCC
GCCCCCGAGGCCGCCGGCGGCCCCAGCGTGTTCCTGTTC
CCCCCCAAGCCCAAGGACACCCTGATGATCAGCCGCACC
CCCGAGGTGACCTGCGTGGTGGTGGACGTGAGCCACGAG
GACCCCGAGGTGAAGTTCAACTGGTACGTGGACGGCGTG
GAGGTGCACAACGCCAAGACCAAGCCCCGCGAGGAGCAG
TACAACAGCACCTACCGCGTGGTGAGCGTGCTGACCGTG
CTGGCCCAGGACTGGCTGAACGGCAAGGAGTACAAGTGC
AAGGTGAGCAACAAGGCCCTGGGCGCCCCCATCGAGAAG
ACCATCAGCAAGGCCAAGGGCCAGCCCCGCGAGCCCCAG
GTGTACACCCTGCCCCCCAGCCGCGACGAGCTGACCAAG
AACCAGGTGAGCCTGACCTGCCTGGTGAAGGGCTTCTAC
CCCAGCGACATCGCCGTGGAGTGGGAGAGCAACGGCCAG
CCCGAGAACAACTACAAGACCACCCCCCCCGTGCTGGAC
AGCGACGGCAGCTTCTTCCTGTACAGCAAGCTGACCGTG
GACAAGAGCCGCTGGCAGCAGGGCAACGTGTTCAGCTGC
AGCGTGATGCACGAGGCCCTGCACAACGCCTACACCCAG
AAGAGCCTGAGCCTGAGCCCCGGCCGCCGCAGCAAGCGC
AGCGGCAGCGGCGCCACCAACTTCAGCCTGCTGAAGCAG
GCCGGCGACGTGGAGGAGAACCCCGGCCCCATGCGCCGC
TTCCTGACCGTGATCAGCTTCCTGCTGTACTTCGGCTGC
GCCTTCGCCGAAGCAACATTTTGTGATTTTCCAAAAATA
AACCATGGAATTCTATATGATGAAGAAAAATATAAGCCA
TTTTCCCAGGTTCCTACAGGGGAAGTTTTCTATTACTCC
TGTGAATATAATTTTGTGTCTCCTTCAAAATCATTTTGG
ACTCGCATAACATGCACAGAAGAAGGATGGTCACCAACA
CCAAAGTGTCTCAGACTGTGTTTCTTTCCTTTTGTGGAA
AATGGTCATTCTGAATCTTCAGGACAAACACATCTGGAA
GGTGATACTGTGCAAATTATTTGCAACACAGGATACAGA
CTTCAAAACAATGAGAACAACATTTCATGTGTAGAACGG
GGCTGGTCCACCCCTCCCAAATGCAGGTCCACTGACACT
TCCTGTGTGAATCCGCCCACAGTACAAAATGCTCATATA
CTGTCGAGACAGATGAGTAAATATCCATCTGGTGAGAGA
GTACGTTATGAATGTAGGAGCCCTTATGAAATGTTTGGG
GATGAAGAAGTGATGTGTTTAAATGGAAACTGGACAGAA
CCACCTCAATGCAAAGATTCTACGGGAAAATGTGGGCCC
CCTCCACCTATTGACAATGGGGACATTACTTCATTCCCG
TTGTCAGTATATGCTCCAGCTTCATCAGTTGAGTACCAA
TGCCAGAACTTGTATCAACTTGAGGGTAACAAGCGAATA
ACATGTAGAAATGGACAATGGTCAGAACCACCAAAATGC
TTACATCCGTGTGTAATATCCCGAGAAATTATGGAAAAT
TATAACATAGCATTAAGGTGGACAGCCAAACAGAAGCTT
TATTTGAGAACAGGTGAATCAGCTGAATTTGTGTGTAAA
CGGGGATATCGTCTTTCATCACGTTCTCACACATTGCGA
ACAACATGTTGGGATGGGAAACTGGAGTATCCAACTTGT
GCAAAAAGATAA

In another embodiment, the amino acid sequence of [CFHR1-furin-P2A-VEGF capture protein 2], is referred to herein as SEQ ID No: 94, or a fragment or variant thereof, as follows:

[SEQ ID No: 94]
MRRFLTVISFLLYFGCAFAEATFCDFPKINHGILYDEEK
YKPFSQVPTGEVFYYSCEYNFVSPSKSFWTRITCTEEGW
SPTPKCLRLCFFPFVENGHSESSGQTHLEGDTVQIICNT
GYRLQNNENNISCVERGWSTPPKCRSTDTSCVNPPTVQN
AHILSRQMSKYPSGERVRYECRSPYEMFGDEEVMCLNGN
WTEPPQCKDSTGKCGPPPPIDNGDITSFPLSVYAPASSV
EYQCQNLYQLEGNKRITCRNGQWSEPPKCLHPCVISREI
MENYNIALRWTAKQKLYLRTGESAEFVCKRGYRLSSRSH
TLRTTCWDGKLEYPTCAKRRRSKRSGSGATNFSLLKQAG
DVEENPGPMRRLLTFISILALVGAAFASDTGRPFVEMYS
EIPEIIHMTEGRELVIPCRVTSPNITVILKKFPLDTLIP
DGKRIIWDSRKGFIISNATYKEIGLLTCEATVNGHLYKT
NYLTHRQTNTIIDVVLSPSHGIELSVGEKLVLNCTARTE
LNVGIDENWEYPSSKHQHKKLVNRDLKTQSGSEMKKFLS
TLTIDGVTRSDQGLYTCAASSGLMTKKNSTFVRVHEKDK
THTCPPCPAPEAAGGPSVFLFPPKPKDTLMISRTPEVTC
VVVDVSHEDPEVKFNWYVDGVEVHNAKTKPREEQYNSTY
RVVSVLTVLAQDWLNGKEYKCKVSNKALGAPIEKTISKA
KGQPREPQVYTLPPSRDELTKNQVSLTCLVKGFYPSDIA
VEWESNGQPENNYKTTPPVLDSDGSFFLYSKLTVDKSRW
QQGNVFSCSVMHEALHNAYTQKSLSLSPG

Preferably, in this embodiment, the construct comprises a 9430 nucleotide sequence (as contained within the plasmid IKC134P), which is referred to herein as SEQ ID No: 95, or a fragment or variant thereof, as follows:

[SEQ ID No: 95]
ATGCGCCGCTTCCTGACCGTGATCAGCTTCCTGCTGTAC
TTCGGCTGCGCCTTCGCCGAAGCAACATTTTGTGATTTT
CCAAAAATAAACCATGGAATTCTATATGATGAAGAAAAA
TATAAGCCATTTTCCCAGGTTCCTACAGGGGAAGTTTTC
TATTACTCCTGTGAATATAATTTTGTGTCTCCTTCAAAA
TCATTTTGGACTCGCATAACATGCACAGAAGAAGGATGG
TCACCAACACCAAAGTGTCTCAGACTGTGTTTCTTTCCT
TTTGTGGAAAATGGTCATTCTGAATCTTCAGGACAAACA
CATCTGGAAGGTGATACTGTGCAAATTATTTGCAACACA
GGATACAGACTTCAAAACAATGAGAACAACATTTCATGT
GTAGAACGGGGCTGGTCCACCCCTCCCAAATGCAGGTCC
ACTGACACTTCCTGTGTGAATCCGCCCACAGTACAAAAT
GCTCATATACTGTCGAGACAGATGAGTAAATATCCATCT
GGTGAGAGAGTACGTTATGAATGTAGGAGCCCTTATGAA
ATGTTTGGGGATGAAGAAGTGATGTGTTTAAATGGAAAC
TGGACAGAACCACCTCAATGCAAAGATTCTACGGGAAAA
TGTGGGCCCCCTCCACCTATTGACAATGGGGACATTACT
TCATTCCCGTTGTCAGTATATGCTCCAGCTTCATCAGTT
GAGTACCAATGCCAGAACTTGTATCAACTTGAGGGTAAC
AAGCGAATAACATGTAGAAATGGACAATGGTCAGAACCA
CCAAAATGCTTACATCCGTGTGTAATATCCCGAGAAATT
ATGGAAAATTATAACATAGCATTAAGGTGGACAGCCAAA
CAGAAGCTTTATTTGAGAACAGGTGAATCAGCTGAATTT
GTGTGTAAACGGGGATATCGTCTTTCATCACGTTCTCAC
ACATTGCGAACAACATGTTGGGATGGGAAACTGGAGTAT
CCAACTTGTGCAAAAAGACGCCGCAGCAAGCGCAGCGGC
AGCGGCGCCACCAACTTCAGCCTGCTGAAGCAGGCCGGC
GACGTGGAGGAGAACCCCGGCCCCATGCGCCGCCTGCTG
ACCTTCATCAGCATCCTGGCCCTGGTGGGCGCCGCCTTC
GCCAGCGACACCGGCCGCCCCTTCGTGGAGATGTACAGC
GAGATCCCCGAGATCATCCACATGACCGAGGGCCGCGAG
CTGGTGATCCCCTGCCGCGTGACCAGCCCCAACATCACC
GTGACCCTGAAGAAGTTCCCCCTGGACACCCTGATCCCC
GACGGCAAGCGCATCATCTGGGACAGCCGCAAGGGCTTC
ATCATCAGCAACGCCACCTACAAGGAGATCGGCCTGCTG
ACCTGCGAGGCCACCGTGAACGGCCACCTGTACAAGACC
AACTACCTGACCCACCGCCAGACCAACACCATCATCGAC
GTGGTGCTGAGCCCCAGCCACGGCATCGAGCTGAGCGTG
GGCGAGAAGCTGGTGCTGAACTGCACCGCCCGCACCGAG
CTGAACGTGGGCATCGACTTCAACTGGGAGTACCCCAGC
AGCAAGCACCAGCACAAGAAGCTGGTGAACCGCGACCTG
AAGACCCAGAGCGGCAGCGAGATGAAGAAGTTCCTGAGC
ACCCTGACCATCGACGGCGTGACCCGCAGCGACCAGGGC
CTGTACACCTGCGCCGCCAGCAGCGGCCTGATGACCAAG
AAGAACAGCACCTTCGTGCGCGTGCACGAGAAGGACAAG
ACCCACACCTGCCCCCCCTGCCCCGCCCCCGAGGCCGCC
GGCGGCCCCAGCGTGTTCCTGTTCCCCCCCAAGCCCAAG
GACACCCTGATGATCAGCCGCACCCCCGAGGTGACCTGC
GTGGTGGTGGACGTGAGCCACGAGGACCCCGAGGTGAAG
TTCAACTGGTACGTGGACGGCGTGGAGGTGCACAACGCC
AAGACCAAGCCCCGCGAGGAGCAGTACAACAGCACCTAC
CGCGTGGTGAGCGTGCTGACCGTGCTGGCCCAGGACTGG
CTGAACGGCAAGGAGTACAAGTGCAAGGTGAGCAACAAG
GCCCTGGGCGCCCCCATCGAGAAGACCATCAGCAAGGCC
AAGGGCCAGCCCCGCGAGCCCCAGGTGTACACCCTGCCC
CCCAGCCGCGACGAGCTGACCAAGAACCAGGTGAGCCTG
ACCTGCCTGGTGAAGGGCTTCTACCCCAGCGACATCGCC
GTGGAGTGGGAGAGCAACGGCCAGCCCGAGAACAACTAC
AAGACCACCCCCCCCGTGCTGGACAGCGACGGCAGCTTC
TTCCTGTACAGCAAGCTGACCGTGGACAAGAGCCGCTGG
CAGCAGGGCAACGTGTTCAGCTGCAGCGTGATGCACGAG
GCCCTGCACAACGCCTACACCCAGAAGAGCCTGAGCCTG
AGCCCCGGCTAA

In another embodiment, the amino acid sequence of [VEGF capture protein 2-furin-P2A-CFHL1], is referred to herein as SEQ ID No: 100, or a fragment or variant thereof, as follows:

[SEQ ID No: 100]
MRRLLTFISILALVGAAFASDTGRPFVEMYSEIPEIIHM
TEGRELVIPCRVTSPNITVTLKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKFLSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLIV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRLLTFISILALVGAFAEDCNELPPRRN
TEILTGSWSDQTYPEGTQAIYKCRPGYRSLGNVIMVCRK
GEWVALNPLRKCQKRPCGHPGDTPFGTFTLTGGNVFEYG
VKAVYTCNEGYQLLGEINYRECDTDGWINDIPICEVVKC
LPVTAPENGKIVSSAMEPDREYHFGQAVRFVCNSGYKIE
GDEEMHCSDDGFWSKEKPKCVEISCKSPDVINGSPISQK
IIYKENERFQYKCNMGYEYSERGDAVCTESGWRPLPSCE
EKSCDNPYIPNGDYSPLRIKHRTGDEITYQCRNGFYPAT
RGNTAKCTSTGWIPAPRCTLKPCDYPDIKHGGLYHENMR
RPYFPVAVGKYYSYYCDEHFETPSGSYWDHIHCTQDGWS
PAVPCLRKCYFPYLENGYNQNYGRKFVQGKSIDVACHPG
YALPKAQTTVTCMENGWSPTPRCIR

Preferably, in this embodiment, the construct comprises a 2769 nucleotide sequence (as contained within the plasmid IKC144P), which is referred to herein as SEQ ID No: 101, or a fragment or variant thereof, as follows:

[SEQ ID No: 101]
ATGCGCCGCCTGCTGACCTTCATCAGCATCCTGGCCCTGG
TGGGCGCCGCCTTCGCCAGCGACACCGGCCGCCCCTTCGTG
GAGATGTACAGCGAGATCCCCGAGATCATCCACATGACCG
AGGGCCGCGAGCTGGTGATCCCCTGCCGCGTGACCAGCCCC
AACATCACCGTGACCCTGAAGAAGTTCCCCCTGGACACCC
TGATCCCCGACGGCAAGCGCATCATCTGGGACAGCCGCAAG
GGCTTCATCATCAGCAACGCCACCTACAAGGAGATCGGCC
TGCTGACCTGCGAGGCCACCGTGAACGGCCACCTGTACAAG
ACCAACTACCTGACCCACCGCCAGACCAACACCATCATCG
ACGTGGTGCTGAGCCCCAGCCACGGCATCGAGCTGAGCGTG
GGCGAGAAGCTGGTGCTGAACTGCACCGCCCGCACCGAGC
TGAACGTGGGCATCGACTTCAACTGGGAGTACCCCAGCAGC
AAGCACCAGCACAAGAAGCTGGTGAACCGCGACCTGAAGA
CCCAGAGCGGCAGCGAGATGAAGAAGTTCCTGAGCACCCTG
ACCATCGACGGCGTGACCCGCAGCGACCAGGGCCTGTACA
CCTGCGCCGCCAGCAGCGGCCTGATGACCAAGAAGAACAGC
ACCTTCGTGCGCGTGCACGAGAAGGACAAGACCCACACCT
GCCCCCCCTGCCCCGCCCCCGAGGCCGCCGGCGGCCCCAGC
GTGTTCCTGTTCCCCCCCAAGCCCAAGGACACCCTGATGA
TCAGCCGCACCCCCGAGGTGACCTGCGTGGTGGTGGACGTG
AGCCACGAGGACCCCGAGGTGAAGTTCAACTGGTACGTGG
ACGGCGTGGAGGTGCACAACGCCAAGACCAAGCCCCGCGAG
GAGCAGTACAACAGCACCTACCGCGTGGTGAGCGTGCTGA
CCGTGCTGGCCCAGGACTGGCTGAACGGCAAGGAGTACAAG
TGCAAGGTGAGCAACAAGGCCCTGGGCGCCCCCATCGAGA
AGACCATCAGCAAGGCCAAGGGCCAGCCCCGCGAGCCCCAG
GTGTACACCCTGCCCCCCAGCCGCGACGAGCTGACCAAGA
ACCAGGTGAGCCTGACCTGCCTGGTGAAGGGCTTCTACCCC
AGCGACATCGCCGTGGAGTGGGAGAGCAACGGCCAGCCCG
AGAACAACTACAAGACCACCCCCCCCGTGCTGGACAGCGAC
GGCAGCTTCTTCCTGTACAGCAAGCTGACCGTGGACAAGA
GCCGCTGGCAGCAGGGCAACGTGTTCAGCTGCAGCGTGATG
CACGAGGCCCTGCACAACGCCTACACCCAGAAGAGCCTGA
GCCTGAGCCCCGGCCGCCGCAGCAAGCGCAGCGGCAGCGGC
GCCACCAACTTCAGCCTGCTGAAGCAGGCCGGCGACGTGG
AGGAGAACCCCGGCCCCATGCGCCGCCTGCTGACCTTCATC
AGCATCCTGGCCCTGGTGGGCGCCTTCGCCGAGGACTGCA
ACGAGCTGCCCCCCCGCCGCAACACCGAGATCCTGACCGGC
AGCTGGAGCGACCAGACCTACCCCGAGGGCACCCAGGCCA
TCTACAAGTGCCGCCCCGGCTACCGCAGCCTGGGCAACGTG
ATCATGGTGTGCCGCAAGGGCGAGTGGGTGGCCCTGAACC
CCCTGCGCAAGTGCCAGAAGCGCCCCTGCGGCCACCCCGGC
GACACCCCCTTCGGCACCTTCACCCTGACCGGCGGCAACG
TGTTCGAGTACGGCGTGAAGGCCGTGTACACCTGCAACGAG
GGCTACCAGCTGCTGGGCGAGATCAACTACCGCGAGTGCG
ACACCGACGGCTGGACCAACGACATCCCCATCTGCGAGGTG
GTGAAGTGCCTGCCCGTGACCGCCCCCGAGAACGGCAAGA
TCGTGAGCAGCGCCATGGAGCCCGACCGCGAGTACCACTTC
GGCCAGGCCGTGCGCTTCGTGTGCAACAGCGGCTACAAGA
TCGAGGGCGACGAGGAGATGCACTGTAGCGACGACGGCTTC
TGGAGCAAGGAGAAGCCCAAGTGCGTGGAGATCAGCTGCA
AGAGCCCCGACGTGATCAACGGCAGCCCCATCAGCCAGAAG
ATCATCTACAAGGAGAACGAGCGCTTCCAGTACAAGTGCA
ACATGGGCTACGAGTACAGCGAGCGCGGCGACGCCGTGTGC
ACCGAGAGCGGCTGGCGCCCCCTGCCCAGCTGCGAGGAGA
AGAGCTGCGACAACCCCTACATCCCCAACGGCGACTACAGC
CCCCTGCGCATCAAGCACCGCACCGGCGACGAGATCACCT
ACCAGTGCCGCAACGGCTTCTACCCCGCCACCCGCGGCAAC
ACCGCCAAGTGCACCAGCACCGGCTGGATCCCCGCCCCCC
GCTGCACCCTGAAGCCCTGCGACTACCCCGACATCAAGCAC
GGCGGCCTGTACCACGAGAACATGCGCCGCCCCTACTTCC
CCGTGGCCGTGGGCAAGTACTACAGCTACTACTGCGACGAG
CACTTCGAGACCCCCAGCGGCAGCTACTGGGACCACATCC
ACTGCACCCAGGACGGCTGGAGCCCCGCCGTGCCCTGCCTG
CGCAAGTGCTACTTCCCCTACCTGGAGAACGGCTACAACC
AGAACTACGGCCGCAAGTTCGTGCAGGGCAAGAGCATCGAC
GTGGCCTGCCACCCCGGCTACGCCCTGCCCAAGGCCCAGA
CCACCGTGACCTGCATGGAGAACGGCTGGAGCCCCACCCCC
CGCTGCATCCGCTAG

In another embodiment, the amino acid sequence of [CFHL1-furin-P2A-VEGF capture protein 2], is referred to herein as SEQ ID No: 102, or a fragment or variant thereof, as follows:

[SEQ ID No: 102]
MRRLLTFISILALVGAFAEDCNELPPRRNTEILTGSWSD
QTYPEGTQAIYKCRPGYRSLGNVIMVCRKGEWVALNPLR
KCQKRPCGHPGDTPFGTFTLTGGNVFEYGVKAVYTCNEG
YQLLGEINYRECDTDGWTNDIPICEVVKCLPVTAPENGK
IVSSAMEPDREYHFGQAVRFVCNSGYKIEGDEEMHCSDD
GFWSKEKPKCVEISCKSPDVINGSPISQKIIYKENERFQ
YKCNMGYEYSERGDAVCTESGWRPLPSCEEKSCDNPYIP
NGDYSPLRIKHRTGDEITYQCRNGFYPATRGNTAKCTST
GWIPAPRCTLKPCDYPDIKHGGLYHENMRRPYFPVAVGK
YYSYYCDEHFETPSGSYWDHIHCTQDGWSPAVPCLRKCY
FPYLENGYNQNYGRKFVQGKSIDVACHPGYALPKAQTTV
TCMENGWSPTPRCIRRRSKRSGSGATNFSLLKQAGDVEE
NPGPMRRLLTFISILALVGAAFASDIGRPFVEMYSEIPE
IIHMTEGRELVIPCRVTSPNITVTLKKFPLDTLIPDGKR
IIWDSRKGFIISNATYKEIGLLTCEATVNGHLYKTNYLT
HRQTNTIIDVVLSPSHGIELSVGEKLVLNCTARTELNVG
IDFNWEYPSSKHQHKKLVNRDLKTQSGSEMKKFLSTLTI
DGVTRSDQGLYTCAASSGLMTKKNSTFVRVHEKDKTHTC
PPCPAPEAAGGPSVFLFPPKPKDTLMISRTPEVTCVVVD
VSHEDPEVKFNWYVDGVEVHNAKTKPREEQYNSTYRVVS
VLTVLAQDWLNGKEYKCKVSNKALGAPIEKTISKAKGQP
REPQVYTLPPSRDELTKNQVSLTCLVKGFYPSDIAVEWE
SNGQPENNYKTTPPVLDSDGSFFLYSKLTVDKSRWQQGN
VFSCSVMHEALHNAYTQKSLSLSPG

Preferably, in this embodiment, the construct comprises a 2766 nucleotide sequence (as contained within the plasmid IKC175P), which is referred to herein as SEQ ID No: 103, or a fragment or variant thereof, as follows:

[SEQ ID No: 103]
ATGCGCCGCCTGCTGACCTTCATCAGCATCCTGGCCCTGG
TGGGCGCCTTCGCCGAGGACTGCAACGAGCTGCCCCCCCGC
CGCAACACCGAGATCCTGACCGGCAGCTGGAGCGACCAGA
CCTACCCCGAGGGCACCCAGGCCATCTACAAGTGCCGCCCC
GGCTACCGCAGCCTGGGCAACGTGATCATGGTGTGCCGCA
AGGGCGAGTGGGTGGCCCTGAACCCCCTGCGCAAGTGCCAG
AAGCGCCCCTGCGGCCACCCCGGCGACACCCCCTTCGGCA
CCTTCACCCTGACCGGCGGCAACGTGTTCGAGTACGGCGTG
AAGGCCGTGTACACCTGCAACGAGGGCTACCAGCTGCTGG
GCGAGATCAACTACCGCGAGTGCGACACCGACGGCTGGACC
AACGACATCCCCATCTGCGAGGTGGTGAAGTGCCTGCCCG
TGACCGCCCCCGAGAACGGCAAGATCGTGAGCAGCGCCATG
GAGCCCGACCGCGAGTACCACTTCGGCCAGGCCGTGCGCT
TCGTGTGCAACAGCGGCTACAAGATCGAGGGCGACGAGGAG
ATGCACTGTAGCGACGACGGCTTCTGGAGCAAGGAGAAGC
CCAAGTGCGTGGAGATCAGCTGCAAGAGCCCCGACGTGATC
AACGGCAGCCCCATCAGCCAGAAGATCATCTACAAGGAGA
ACGAGCGCTTCCAGTACAAGTGCAACATGGGCTACGAGTAC
AGCGAGCGCGGCGACGCCGTGTGCACCGAGAGCGGCTGGC
GCCCCCTGCCCAGCTGCGAGGAGAAGAGCTGCGACAACCCC
TACATCCCCAACGGCGACTACAGCCCCCTGCGCATCAAGC
ACCGCACCGGCGACGAGATCACCTACCAGTGCCGCAACGGC
TTCTACCCCGCCACCCGCGGCAACACCGCCAAGTGCACCA
GCACCGGCTGGATCCCCGCCCCCCGCTGCACCCTGAAGCCC
TGCGACTACCCCGACATCAAGCACGGCGGCCTGTACCACG
AGAACATGCGCCGCCCCTACTTCCCCGTGGCCGTGGGCAAG
TACTACAGCTACTACTGCGACGAGCACTTCGAGACCCCCA
GCGGCAGCTACTGGGACCACATCCACTGCACCCAGGACGGC
TGGAGCCCCGCCGTGCCCTGCCTGCGCAAGTGCTACTTCC
CCTACCTGGAGAACGGCTACAACCAGAACTACGGCCGCAAG
TTCGTGCAGGGCAAGAGCATCGACGTGGCCTGCCACCCCG
GCTACGCCCTGCCCAAGGCCCAGACCACCGTGACCTGCATG
GAGAACGGCTGGAGCCCCACCCCCCGCTGCATCCGCCGCC
GCAGCAAGCGCAGCGGCAGCGGCGCCACCAACTTCAGCCTG
CTGAAGCAGGCCGGCGACGTGGAGGAGAACCCCGGCCCCA
TGCGCCGCCTGCTGACCTTCATCAGCATCCTGGCCCTGGTG
GGCGCCGCCTTCGCCAGCGACACCGGCCGCCCCTTCGTGG
AGATGTACAGCGAGATCCCCGAGATCATCCACATGACCGAG
GGCCGCGAGCTGGTGATCCCCTGCCGCGTGACCAGCCCCA
ACATCACCGTGACCCTGAAGAAGTTCCCCCTGGACACCCTG
ATCCCCGACGGCAAGCGCATCATCTGGGACAGCCGCAAGG
GCTTCATCATCAGCAACGCCACCTACAAGGAGATCGGCCTG
CTGACCTGCGAGGCCACCGTGAACGGCCACCTGTACAAGA
CCAACTACCTGACCCACCGCCAGACCAACACCATCATCGAC
GTGGTGCTGAGCCCCAGCCACGGCATCGAGCTGAGCGTGG
GCGAGAAGCTGGTGCTGAACTGCACCGCCCGCACCGAGCTG
AACGTGGGCATCGACTTCAACTGGGAGTACCCCAGCAGCA
AGCACCAGCACAAGAAGCTGGTGAACCGCGACCTGAAGACC
CAGAGCGGCAGCGAGATGAAGAAGTTCCTGAGCACCCTGA
CCATCGACGGCGTGACCCGCAGCGACCAGGGCCTGTACACC
TGCGCCGCCAGCAGCGGCCTGATGACCAAGAAGAACAGCA
CCTTCGTGCGCGTGCACGAGAAGGACAAGACCCACACCTGC
CCCCCCTGCCCCGCCCCCGAGGCCGCCGGCGGCCCCAGCG
TGTTCCTGTTCCCCCCCAAGCCCAAGGACACCCTGATGATC
AGCCGCACCCCCGAGGTGACCTGCGTGGTGGTGGACGTGA
GCCACGAGGACCCCGAGGTGAAGTTCAACTGGTACGTGGAC
GGCGTGGAGGTGCACAACGCCAAGACCAAGCCCCGCGAGG
AGCAGTACAACAGCACCTACCGCGTGGTGAGCGTGCTGACC
GTGCTGGCCCAGGACTGGCTGAACGGCAAGGAGTACAAGT
GCAAGGTGAGCAACAAGGCCCTGGGCGCCCCCATCGAGAAG
ACCATCAGCAAGGCCAAGGGCCAGCCCCGCGAGCCCCAGG
TGTACACCCTGCCCCCCAGCCGCGACGAGCTGACCAAGAAC
CAGGTGAGCCTGACCTGCCTGGTGAAGGGCTTCTACCCCA
GCGACATCGCCGTGGAGTGGGAGAGCAACGGCCAGCCCGAG
AACAACTACAAGACCACCCCCCCCGTGCTGGACAGCGACG
GCAGCTTCTTCCTGTACAGCAAGCTGACCGTGGACAAGAGC
CGCTGGCAGCAGGGCAACGTGTTCAGCTGCAGCGTGATGC
ACGAGGCCCTGCACAACGCCTACACCCAGAAGAGCCTGAG
CCTGAGCCCCGGC

In another embodiment, the amino acid sequence of [VEGF capture protein 2-furin-P2A-sCD46], is referred to herein as SEQ ID No: 104, or a fragment or variant thereof, as follows:

[SEQ ID No: 104]
MRRLLTFISILALVGAAFASDTGRPFVEMYSEIPEIIHM
TEGRELVIPCRVTSPNITVTLKKFPLDTLIPDGKRIIWD
SRKGFIISNATYKEIGLLTCEATVNGHLYKTNYLTHRQT
NTIIDVVLSPSHGIELSVGEKLVLNCTARTELNVGIDFN
WEYPSSKHQHKKLVNRDLKTQSGSEMKKFLSTLTIDGVT
RSDQGLYTCAASSGLMTKKNSTFVRVHEKDKTHTCPPCP
APEAAGGPSVFLFPPKPKDTLMISRTPEVTCVVVDVSHE
DPEVKFNWYVDGVEVHNAKTKPREEQYNSTYRVVSVLTV
LAQDWLNGKEYKCKVSNKALGAPIEKTISKAKGQPREPQ
VYTLPPSRDELTKNQVSLTCLVKGFYPSDIAVEWESNGQ
PENNYKTTPPVLDSDGSFFLYSKLTVDKSRWQQGNVFSC
SVMHEALHNAYTQKSLSLSPGRRSKRSGSGATNFSLLKQ
AGDVEENPGPMRRFLTVISFLLYFGCAFACEEPPTFEAM
ELIGKPKPYYEIGERVDYKCKKGYFYIPPLATHTICDRN
HTWLPVSDDACYRETCPYIRDPLNGQAVPANGTYEFGYQ
MHFICNEGYYLIGEEILYCELKGSVAIWSGKPPICEKVL
CTPPPKIKNGKHTFSEVEVFEYLDAVTYSCDPAPGPDPF
SLIGESTIYCGDNSVWSRAAPECKVVKCRFPVVENGKQI
SGFGKKFYYKATVMFECDKGFYLDGSDTIVCDSNSTWDP
PVPKCLKVLPPSSTKPPALSHSVSTSSTTKSPASSASGP
RPTYKPPVSNYPGYPKPEEGILDSLDV

Preferably, in this embodiment, the construct comprises a 2424 nucleotide sequence (as contained within the plasmid IKC176P), which is referred to herein as SEQ ID No: 105, or a fragment or variant thereof, as follows:

[SEQ ID No: 105]
ATGCGCCGCCTGCTGACCTTCATCAGCATCCTGGCCCTGG
TGGGCGCCGCCTTCGCCAGCGACACCGGCCGCCCCTTCGTG
GAGATGTACAGCGAGATCCCCGAGATCATCCACATGACCG
AGGGCCGCGAGCTGGTGATCCCCTGCCGCGTGACCAGCCCC
AACATCACCGTGACCCTGAAGAAGTTCCCCCTGGACACCC
TGATCCCCGACGGCAAGCGCATCATCTGGGACAGCCGCAAG
GGCTTCATCATCAGCAACGCCACCTACAAGGAGATCGGCC
TGCTGACCTGCGAGGCCACCGTGAACGGCCACCTGTACAAG
ACCAACTACCTGACCCACCGCCAGACCAACACCATCATCG
ACGTGGTGCTGAGCCCCAGCCACGGCATCGAGCTGAGCGTG
GGCGAGAAGCTGGTGCTGAACTGCACCGCCCGCACCGAGC
TGAACGTGGGCATCGACTTCAACTGGGAGTACCCCAGCAGC
AAGCACCAGCACAAGAAGCTGGTGAACCGCGACCTGAAGA
CCCAGAGCGGCAGCGAGATGAAGAAGTTCCTGAGCACCCTG
ACCATCGACGGCGTGACCCGCAGCGACCAGGGCCTGTACA
CCTGCGCCGCCAGCAGCGGCCTGATGACCAAGAAGAACAGC
ACCTTCGTGCGCGTGCACGAGAAGGACAAGACCCACACCT
GCCCCCCCTGCCCCGCCCCCGAGGCCGCCGGCGGCCCCAGC
GTGTTCCTGTTCCCCCCCAAGCCCAAGGACACCCTGATGA
TCAGCCGCACCCCCGAGGTGACCTGCGTGGTGGTGGACGTG
AGCCACGAGGACCCCGAGGTGAAGTTCAACTGGTACGTGG
ACGGCGTGGAGGTGCACAACGCCAAGACCAAGCCCCGCGAG
GAGCAGTACAACAGCACCTACCGCGTGGTGAGCGTGCTGA
CCGTGCTGGCCCAGGACTGGCTGAACGGCAAGGAGTACAAG
TGCAAGGTGAGCAACAAGGCCCTGGGCGCCCCCATCGAGA
AGACCATCAGCAAGGCCAAGGGCCAGCCCCGCGAGCCCCAG
GTGTACACCCTGCCCCCCAGCCGCGACGAGCTGACCAAGA
ACCAGGTGAGCCTGACCTGCCTGGTGAAGGGCTTCTACCCC
AGCGACATCGCCGTGGAGTGGGAGAGCAACGGCCAGCCCG
AGAACAACTACAAGACCACCCCCCCCGTGCTGGACAGCGAC
GGCAGCTTCTTCCTGTACAGCAAGCTGACCGTGGACAAGA
GCCGCTGGCAGCAGGGCAACGTGTTCAGCTGCAGCGTGATG
CACGAGGCCCTGCACAACGCCTACACCCAGAAGAGCCTGA
GCCTGAGCCCCGGCCGCCGCAGCAAGCGCAGCGGCAGCGGC
GCCACCAACTTCAGCCTGCTGAAGCAGGCCGGCGACGTGG
AGGAGAACCCCGGCCCCATGCGCCGCTTCCTGACCGTGATC
AGCTTCCTGCTGTACTTCGGCTGCGCCTTCGCCTGCGAGG
AGCCCCCCACCTTCGAGGCCATGGAGCTGATCGGCAAGCCC
AAGCCCTACTACGAGATCGGCGAGCGCGTGGACTACAAGT
GCAAGAAGGGCTACTTCTACATCCCCCCCCTGGCCACCCAC
ACCATCTGCGACCGCAACCACACCTGGCTGCCCGTGAGCG
ACGACGCCTGCTACCGCGAGACCTGCCCCTACATCCGCGAC
CCCCTGAACGGCCAGGCCGTGCCCGCCAACGGCACCTACG
AGTTCGGCTACCAGATGCACTTCATCTGCAACGAGGGCTAC
TACCTGATCGGCGAGGAGATCCTGTACTGCGAGCTGAAGG
GCAGCGTGGCCATCTGGAGCGGCAAGCCCCCCATCTGCGAG
AAGGTGCTGTGCACCCCCCCCCCCAAGATCAAGAACGGCA
AGCACACCTTCAGCGAGGTGGAGGTGTTCGAGTACCTGGAC
GCCGTGACCTACAGCTGCGACCCCGCCCCCGGCCCCGACC
CCTTCAGCCTGATCGGCGAGAGCACCATCTACTGCGGCGAC
AACAGCGTGTGGAGCCGCGCCGCCCCCGAGTGCAAGGTGG
TGAAGTGCCGCTTCCCCGTGGTGGAGAACGGCAAGCAGATC
AGCGGCTTCGGCAAGAAGTTCTACTACAAGGCCACCGTGA
TGTTCGAGTGCGACAAGGGCTTCTACCTGGACGGCAGCGAC
ACCATCGTGTGCGACAGCAACAGCACCTGGGACCCCCCCG
TGCCCAAGTGCCTGAAGGTGCTGCCCCCCAGCAGCACCAAG
CCCCCCGCCCTGAGCCACAGCGTGAGCACCAGCAGCACCA
CCAAGAGCCCCGCCAGCAGCGCCAGCGGCCCCCGCCCCACC
TACAAGCCCCCCGTGAGCAACTACCCCGGCTACCCCAAGC
CCGAGGAGGGCATCCTGGACAGCCTGGACGTGTAA

In another embodiment, the amino acid sequence of [sCD46-furin-P2A-VEGF capture protein 2], is referred to herein as SEQ ID No: 106, or a fragment or variant thereof, as follows:

[SEQ ID No: 106]
MRRFLTVISFLLYFGCAFACEEPPTFEAMELIGKPKPYY
EIGERVDYKCKKGYFYIPPLATHTICDRNHTWLPVSDDA
CYRETCPYIRDPLNGQAVPANGTYEFGYQMHFICNEGYY
LIGEEILYCELKGSVAIWSGKPPICEKVLCTPPPKIKNG
KHTFSEVEVFEYLDAVTYSCDPAPGPDPFSLIGESTIYC
GDNSVWSRAAPECKVVKCRFPVVENGKQISGFGKKFYYK
ATVMFECDKGFYLDGSDTIVCDSNSTWDPPVPKCLKVLP
PSSTKPPALSHSVSTSSTTKSPASSASGPRPTYKPPVSN
YPGYPKPEEGILDSLDVRRSKRSGSGATNFSLLKQAGDV
EENPGPMRRLLTFISILALVGAAFASDTGRPFVEMYSEI
PEIIHMTEGRELVIPCRVTSPNITVILKKFPLDTLIPDG
KRIIWDSRKGFIISNATYKEIGLLTCEATVNGHLYKTNY
LTHRQTNTIIDVVLSPSHGIELSVGEKLVLNCTARTELN
VGIDENWEYPSSKHQHKKLVNRDLKTQSGSEMKKFLSTL
TIDGVTRSDQGLYTCAASSGLMTKKNSTFVRVHEKDKTH
TCPPCPAPEAAGGPSVFLFPPKPKDTLMISRTPEVTCVV
VDVSHEDPEVKFNWYVDGVEVHNAKTKPREEQYNSTYRV
VSVLIVLAQDWLNGKEYKCKVSNKALGAPIEKTISKAKG
QPREPQVYTLPPSRDELTKNQVSLTCLVKGFYPSDIAVE
WESNGQPENNYKTTPPVLDSDGSFFLYSKLTVDKSRWQQ
GNVFSCSVMHEALHNAYTQKSLSLSPG

Preferably, in this embodiment, the construct comprises a 2421 nucleotide sequence (as contained within the plasmid IKC177P), which is referred to herein as SEQ ID No: 107, or a fragment or variant thereof, as follows:

[SEQ ID No: 107]
ATGCGCCGCTTCCTGACCGTGATCAGCTTCCTGCTGTACT
TCGGCTGCGCCTTCGCCTGCGAGGAGCCCCCCACCTTCGAG
GCCATGGAGCTGATCGGCAAGCCCAAGCCCTACTACGAGA
TCGGCGAGCGCGTGGACTACAAGTGCAAGAAGGGCTACTTC
TACATCCCCCCCCTGGCCACCCACACCATCTGCGACCGCA
ACCACACCTGGCTGCCCGTGAGCGACGACGCCTGCTACCGC
GAGACCTGCCCCTACATCCGCGACCCCCTGAACGGCCAGG
CCGTGCCCGCCAACGGCACCTACGAGTTCGGCTACCAGATG
CACTTCATCTGCAACGAGGGCTACTACCTGATCGGCGAGG
AGATCCTGTACTGCGAGCTGAAGGGCAGCGTGGCCATCTGG
AGCGGCAAGCCCCCCATCTGCGAGAAGGTGCTGTGCACCC
CCCCCCCCAAGATCAAGAACGGCAAGCACACCTTCAGCGAG
GTGGAGGTGTTCGAGTACCTGGACGCCGTGACCTACAGCT
GCGACCCCGCCCCCGGCCCCGACCCCTTCAGCCTGATCGGC
GAGAGCACCATCTACTGCGGCGACAACAGCGTGTGGAGCC
GCGCCGCCCCCGAGTGCAAGGTGGTGAAGTGCCGCTTCCCC
GTGGTGGAGAACGGCAAGCAGATCAGCGGCTTCGGCAAGA
AGTTCTACTACAAGGCCACCGTGATGTTCGAGTGCGACAAG
GGCTTCTACCTGGACGGCAGCGACACCATCGTGTGCGACA
GCAACAGCACCTGGGACCCCCCCGTGCCCAAGTGCCTGAAG
GTGCTGCCCCCCAGCAGCACCAAGCCCCCCGCCCTGAGCC
ACAGCGTGAGCACCAGCAGCACCACCAAGAGCCCCGCCAGC
AGCGCCAGCGGCCCCCGCCCCACCTACAAGCCCCCCGTGA
GCAACTACCCCGGCTACCCCAAGCCCGAGGAGGGCATCCTG
GACAGCCTGGACGTGCGCCGCAGCAAGCGCAGCGGCAGCG
GCGCCACCAACTTCAGCCTGCTGAAGCAGGCCGGCGACGTG
GAGGAGAACCCCGGCCCCATGCGCCGCCTGCTGACCTTCA
TCAGCATCCTGGCCCTGGTGGGCGCCGCCTTCGCCAGCGAC
ACCGGCCGCCCCTTCGTGGAGATGTACAGCGAGATCCCCG
AGATCATCCACATGACCGAGGGCCGCGAGCTGGTGATCCCC
TGCCGCGTGACCAGCCCCAACATCACCGTGACCCTGAAGA
AGTTCCCCCTGGACACCCTGATCCCCGACGGCAAGCGCATC
ATCTGGGACAGCCGCAAGGGCTTCATCATCAGCAACGCCA
CCTACAAGGAGATCGGCCTGCTGACCTGCGAGGCCACCGTG
AACGGCCACCTGTACAAGACCAACTACCTGACCCACCGCC
AGACCAACACCATCATCGACGTGGTGCTGAGCCCCAGCCAC
GGCATCGAGCTGAGCGTGGGCGAGAAGCTGGTGCTGAACT
GCACCGCCCGCACCGAGCTGAACGTGGGCATCGACTTCAAC
TGGGAGTACCCCAGCAGCAAGCACCAGCACAAGAAGCTGG
TGAACCGCGACCTGAAGACCCAGAGCGGCAGCGAGATGAAG
AAGTTCCTGAGCACCCTGACCATCGACGGCGTGACCCGCA
GCGACCAGGGCCTGTACACCTGCGCCGCCAGCAGCGGCCTG
ATGACCAAGAAGAACAGCACCTTCGTGCGCGTGCACGAGA
AGGACAAGACCCACACCTGCCCCCCCTGCCCCGCCCCCGAG
GCCGCCGGCGGCCCCAGCGTGTTCCTGTTCCCCCCCAAGC
CCAAGGACACCCTGATGATCAGCCGCACCCCCGAGGTGACC
TGCGTGGTGGTGGACGTGAGCCACGAGGACCCCGAGGTGA
AGTTCAACTGGTACGTGGACGGCGTGGAGGTGCACAACGCC
AAGACCAAGCCCCGCGAGGAGCAGTACAACAGCACCTACC
GCGTGGTGAGCGTGCTGACCGTGCTGGCCCAGGACTGGCTG
AACGGCAAGGAGTACAAGTGCAAGGTGAGCAACAAGGCCC
TGGGCGCCCCCATCGAGAAGACCATCAGCAAGGCCAAGGGC
CAGCCCCGCGAGCCCCAGGTGTACACCCTGCCCCCCAGCC
GCGACGAGCTGACCAAGAACCAGGTGAGCCTGACCTGCCTG
GTGAAGGGCTTCTACCCCAGCGACATCGCCGTGGAGTGGG
AGAGCAACGGCCAGCCCGAGAACAACTACAAGACCACCCCC
CCCGTGCTGGACAGCGACGGCAGCTTCTTCCTGTACAGCA
AGCTGACCGTGGACAAGAGCCGCTGGCAGCAGGGCAACGTG
TTCAGCTGCAGCGTGATGCACGAGGCCCTGCACAACGCCT
ACACCCAGAAGAGCCTGAGCCTGAGCCCCGGC

Hence, in a preferred embodiment, the construct encodes an amino acid sequence substantially as set out in SEQ ID No: 80, 82, 84, 86, 88, 90, 92, 94, 100, 102, 104 or 106, or a fragment or variant thereof.

Preferably, the construct comprises a nucleotide sequence substantially as set out in SEQ ID No: 81, 83, 85, 87, 89, 91, 93, 95, 101, 103, 105 or 107, or a fragment or variant thereof.

The inventors have created a series of recombinant expression vectors comprising the construct of the invention.

Thus, according to a second aspect, there is provided a recombinant vector comprising the genetic construct according to the first aspect.

In one embodiment, the recombinant vector (e.g. known as “IKC153P”) comprises a nucleotide sequence which is referred to herein as SEQ ID No: 96, or a fragment or variant thereof, as follows:

[SEQ ID No: 96]
CGCGCTCGCTCGCTCACTGAGGCCGCCCGGGCAAAGCCC
GGGCGGCCTCAGTGAGCGAGCGAGCGCGCAGAGAGGGAG
TGGCCAACTCCATCACTAGGGGTTCCTTGTAGTTAATGA
TTAACCCGCCATGCTACTTATCTACGTAGCCATGCTCTA
GACATGGCTCGACAGATCGAGCTCCACCGGGTACCCTAG
TTATTAATAGTAATCAATTACGGGGTCATTAGTTCATAG
CCCATATATGGAGTTCCGCGTTACATAACTTACGGTAAA
TGGCCCGCCTGGCTGACCGCCCAACGACCCCCGCCCATT
GACGTCAATAATGACGTATGTTCCCATAGTAACGCCAAT
AGGGACTTTCCATTGACGTCAATGGGTGGACTATTTACG
GTAAACTGCCCACTTGGCAGTACATCAAGTGTATCATAT
GCCAAGTACGCCCCCTATTGACGTCAATGACGGTAAATG
GCCCGCCTGGCATTATGCCCAGTACATGACCTTATGGGA
CTTTCCTACTTGGCAGTACATCTACGTATTAGTCATCGC
TATTACCATGGTCGAGGTGAGCCCCACGTTCTGCTTCAC
TCTCCCCATCTCCCCCCCCTCCCCACCCCCAATTTTGTA
TTTATTTATTTTTTAATTATTTTGTGCAGCGATGGGGGC
GGGGGGGGGGGGGGGGCGCGCGCCAGGCGGGGCGGGGCG
GGGCGAGGGGCGGGGCGGGGCGAGGCGGAGAGGTGCGGC
GGCAGCCAATCAGAGCGGCGCGCTCCGAAAGTTTCCTTT
TATGGCGAGGCGGCGGCGGCGGCGGCCCTATAAAAAGCG
AAGCGCGCGGCGGGCGGGAGTCGCTGCGACGCTGCCTTC
GCCCCGTGCCCCGCTCCGCCGCCGCCTCGCGCCGCCCGC
CCCGGCTCTGACTGACCGCGTTACTCCCACAGGTGAGCG
GGCGGGACGGCCCTTCTCCTCCGGGCTGTAATTAGCGCT
TGGTTTAATGACGGCTTGTTTCTTTTCTGTGGCTGCGTG
AAAGCCTTGAGGGGCTCCGGGAGGCTAGAGCCTCTGCTA
ACCATGTTCATGCCTTCTTCTTTTTCCTACAGCTCCTGG
GCAACGTGCTGGTTATTGTGCTGTCTCATCATTTTGGCA
AAGAATTCCGGACTCGACCCGCTAGCGCCACCATGCGCC
GCCTGCTGACCTTCATCAGCATCCTGGCCCTGGTGGGCG
CCGCCTTCGCCAGCGACACCGGCCGCCCCTTCGTGGAGA
TGTACAGCGAGATCCCCGAGATCATCCACATGACCGAGG
GCCGCGAGCTGGTGATCCCCTGCCGCGTGACCAGCCCCA
ACATCACCGTGACCCTGAAGAAGTTCCCCCTGGACACCC
TGATCCCCGACGGCAAGCGCATCATCTGGGACAGCCGCA
AGGGCTTCATCATCAGCAACGCCACCTACAAGGAGATCG
GCCTGCTGACCTGCGAGGCCACCGTGAACGGCCACCTGT
ACAAGACCAACTACCTGACCCACCGCCAGACCAACACCA
TCATCGACGTGGTGCTGAGCCCCAGCCACGGCATCGAGC
TGAGCGTGGGCGAGAAGCTGGTGCTGAACTGCACCGCCC
GCACCGAGCTGAACGTGGGCATCGACTTCAACTGGGAGT
ACCCCAGCAGCAAGCACCAGCACAAGAAGCTGGTGAACC
GCGACCTGAAGACCCAGAGCGGCAGCGAGATGAAGAAGT
TCCTGAGCACCCTGACCATCGACGGCGTGACCCGCAGCG
ACCAGGGCCTGTACACCTGCGCCGCCAGCAGCGGCCTGA
TGACCAAGAAGAACAGCACCTTCGTGCGCGTGCACGAGA
AGGACAAGACCCACACCTGCCCCCCCTGCCCCGCCCCCG
AGGCCGCCGGCGGCCCCAGCGTGTTCCTGTTCCCCCCCA
AGCCCAAGGACACCCTGATGATCAGCCGCACCCCCGAGG
TGACCTGCGTGGTGGTGGACGTGAGCCACGAGGACCCCG
AGGTGAAGTTCAACTGGTACGTGGACGGCGTGGAGGTGC
ACAACGCCAAGACCAAGCCCCGCGAGGAGCAGTACAACA
GCACCTACCGCGTGGTGAGCGTGCTGACCGTGCTGGCCC
AGGACTGGCTGAACGGCAAGGAGTACAAGTGCAAGGTGA
GCAACAAGGCCCTGGGCGCCCCCATCGAGAAGACCATCA
GCAAGGCCAAGGGCCAGCCCCGCGAGCCCCAGGTGTACA
CCCTGCCCCCCAGCCGCGACGAGCTGACCAAGAACCAGG
TGAGCCTGACCTGCCTGGTGAAGGGCTTCTACCCCAGCG
ACATCGCCGTGGAGTGGGAGAGCAACGGCCAGCCCGAGA
ACAACTACAAGACCACCCCCCCCGTGCTGGACAGCGACG
GCAGCTTCTTCCTGTACAGCAAGCTGACCGTGGACAAGA
GCCGCTGGCAGCAGGGCAACGTGTTCAGCTGCAGCGTGA
TGCACGAGGCCCTGCACAACGCCTACACCCAGAAGAGCC
TGAGCCTGAGCCCCGGCCGCCGCAGCAAGCGCAGCGGCA
GCGGCGCCACCAACTTCAGCCTGCTGAAGCAGGCCGGCG
ACGTGGAGGAGAACCCCGGCCCCATGCGCCGCTTCCTGA
CCGTGATCAGCTTCCTGCTGTACTTCGGCTGCGCCTTCG
CCGACATCCAGATGACCCAGAGCCCCAGCAGCCTGAGCG
CCAGCGTGGGCGACCGCGTGACCATCACCTGCCGCGCCA
GCCAGGGCATCAGCAGCTGGCTGGCCTGGTACCAGCAGA
AGCCCGAGAAGGCCCCCAAGAGCCTGATCTACGCCGCCA
GCAGCCTCCAGAGCGGCGTGCCCAGCCGCTTCAGCGGCA
GCGGCAGCGGCACCGACTTCACCCTGACCATCAGCAGCC
TCCAGCCCGAGGACTTCGCCACCTACTACTGCCAGCAGT
ACAACAGCTACCCCCCCACCTTCGGCCAGGGCACCAAGC
TGGAGATCAAGCGCGGCGGCGGCGGCGGCAGCGGCGGCG
GCGGCAGCGGCGGCGGCGGCAGCGGCGGCGGCGGCAGCG
AGGGCCAGCTGGTGCAGAGCGGCGGCGGCCTGGTGCACC
CCGGCGGCAGCCTGCGCCTGAGCTGCGCCGGCAGCGGCT
TCACCTTCAGCAGCTACGGCATGCACTGGGTGCGCCAGG
CCCCCGGCAAGGGCCTGGAGTGGGTGAGCGGCATCGGCA
CCGGCGGCGGCACCTACAGCACCGACAGCGTGAAGGGCC
GCTTCACCATCAGCCGCGACAACGCCAAGAACAGCCTGT
ACCTCCAGATGAACAGCCTGCGCGCCGAGGACATGGCCG
TGTACTACTGCGCCCGCGGCGACTACTACGGCAGCGGCA
GCTTCTTCGACTGCTGGGGCCAGGGCACCCTGGTGACCG
TGAGCAGCTAAGTATACTACTAGTACGCGGCCGCACCGG
TGTACAATCAACCTCTGGATTACAAAATTTGTGAAAGAT
TGACTGGTATTCTTAACTATGTTGCTCCTTTTACGCTAT
GTGGATACGCTGCTTTAATGCCTTTGTATCATGCTATTG
CTTCCCGTATGGCTTTCATTTTCTCCTCCTTGTATAAAT
CCTGGTTAGTTCTTGCCACGGCGGAACTCATCGCCGCCT
GCCTTGCCCGCTGCTGGACAGGGGCTCGGCTGTTGGGCA
CTGACAATTCCGTGGTGTGAATTCGAGCTAGGTACAGCT
TATCGATACCGTCGACAGCAGACATGATAAGATACATTG
ATGAGTTTGGACAAACCACAACTAGAATGCAGTGAAAAA
AATGCTTTATTTGTGAAATTTGTGATGCTATTGCTTTAT
TTGTAACCATTATAAGCTGCAATAAACAAGTTAACAACA
ACAATTGCATTCATTTTATGTTTCAGGTTCAGGGGGAGG
TGTGGGAGGTTTTTTAAAGCAAGTAAAACCTCTACAAAT
GTGGTATGCTCGAGGGCATGCAACAACAACAATTGCATT
CATTTTATGTTTCAGGTTCAGGGGGAGATGTGGGAGGTT
TTTTAAAGCAAGTAAAACCTCTACAAATGTGGTAAAATC
CGATAAGGACTAGAGCATGGCTACGTAGATAAGTAGCAT
GGCGGGTTAATCATTAACTACAAGGAACCCCTAGTGATG
GAGTTGGCCACTCCCTCTCTGCGCGCTCGCTCGCTCACT
GAGGCCGGGCGACCAAAGGTCGCCCGACGCCCGGGCTTT
GCCCGGGCGGCCTCAGTGAGCGAGCGAGCGCGCA

Accordingly, in one embodiment, the recombinant vector comprises a nucleotide sequence substantially as set out in SEQ ID No: 96, or a fragment or variant thereof.

The recombinant vector may be a recombinant AAV (rAAV) vector. The rAAV may be a naturally occurring vector or a vector with a hybrid AAV serotype. The rAAV may be AAV-1, AAV-2, AAV-2.7m8, AAV-3A, AAV-3B, AAV-4, AAV-5, AAV-6, AAV-7, AAV-8, AAV-9, AAV-10, and AAV-11. Preferably, the rAAV is rAAV serotype-2.

Advantageously, recombinant AAV2 evokes a minimal immune response in host organisms and mediates long-term transgene expression that can persist in the retina for at least one year after vector administration.

The term “recombinant AAV (rAAV) vector” can mean a recombinant AAV-derived nucleic acid. It may contain at least one terminal repeat sequence.

The capsid coat of the AAV and recombinant vectors are known to be composed of three capsid proteins called VP1, VP2 and VP3, which all comprise a significant amount of overlapping amino acids between them, but unique N-terminal sequences. The AAV virus contains 60 subunits with a 1:1:10 ratio of each of the VP1, VP2 and VP3 capsid proteins, which together form an icosahedral structure. Many AAV serotypes have been identified which differ in the amino acid compositions and thus confer different binding properties to receptors on host cells. There are several naturally occurring AAV serotypes identified, such as AAV1, AAV2, AAV2.7m8, AAV3, AAV4, AAV5, AAV6, AA7, AAV8, AAV9, AAV10, AAV11 and AAV12 and many artificial variants in which further modifications to the amino acid sequence have been identified through screening DNA variants from libraries of capsid coding sequences. Different serotypes can therefore display tropism and exchanging various amino acids of one pseudotype can change the tropism or infectivity for different target cells. Thus, specific AAV pseudotypes can be designed to target one particular cell type or greatly restrict infectivity to a particular organ. In some embodiments, an rAAV vector is a vector derived from an AAV serotype, including AAV1, AAV2, AAV2.7m8, AAV3, AAV4, AAV5, AAV6, AA7, AAV8, AAV9, AAV10, AAV11, or AAV12. An rAAV particle may comprise a capsid protein derived from any AAV serotype, including AAV1, AAV2, AAV2.7m8, AAV3, AAV4, AAV5, AAV6, AA7, AAV8, AAV9, AAV10, AAVrh10, AAV11, or AAV12 capsid. An rAAV particle can comprise viral proteins and viral nucleic acids of the same serotype or a mixed serotype.

A capsid protein or a recombinant viral particle of the invention may comprise or consist of an amino acid sequence of a naturally occurring protein (for example, a naturally occurring AAV capsid protein, such as capsid protein of AAV serotype AAV1, AAV2, AAV2.7m8, AAV3, AAV4, AAV5, AAV6, AAV7, AAV8, AAV9, AAV10, AAVrh10, AAV11, or AAV12), or may be a derivative or chimera of a naturally occurring capsid protein that includes one or more amino acid substitutions, deletions, or additions, compared to the amino acid sequence of the naturally occurring capsid protein, for example to confer tropism for a desired tissue type or cell type (such as retinal ganglion cell, photoreceptor or retinal pigment epithelial cell), or to reduce immunogenicity of the recombinant virus particle.

In some embodiments a capsid protein of a recombinant virus particle of the invention has an amino acid sequence that has at least 60%, 70%, 80%, 90%, 95%, 96%, 97%, 98% or 99% amino acid identity along its entire length to the amino acid sequence of a naturally occurring capsid protein, for example a naturally occurring AAV capsid protein of serotype AAV1, AAV2, AAV2.7m8, AAV3, AAV4, AAV5, AAV6, AA7, AAV8, AAV9, AAV10, AAVrh10, AAV11, or AAV12.

The constructs and expression vectors described herein can be used to treat retinal disorders, particularly wet age-related macular degeneration, or diabetic retinopathies including diabetic macular oedema (DMO), and more generally reduce vascular leakage and retinal cell damage.

Hence, according to a third aspect, there is provided the genetic construct according to the first aspect, or the recombinant vector according to the second aspect, for use as a medicament or in therapy.

According to a fourth aspect, there is provided the genetic construct according to the first aspect, or the recombinant vector according to the second aspect, for use in treating, preventing or ameliorating a retinal disorder, or for reducing vascular leakage and retinal cell damage.

According to a fifth aspect, there is provided a method of treating, preventing or ameliorating a retinal disorder in a subject, or for reducing vascular leakage and retinal cell damage, the method comprising administering, or having administered, to a subject in need of such treatment, a therapeutically effective amount of the genetic construct according to the first aspect, or the recombinant vector according to the second aspect.

Preferably, the genetic construct or the recombinant vector according to invention are used in a gene therapy technique. The anti-VEGF protein and anti-fibrotic protein encoded by the construct or vector neutralise both VEGF and either CTGF or complement proteins, to thereby prevent neovascularisation, reduce vascular leakage, and reduce inflammation, fibrosis and scarring.

In one embodiment, the retinal disorder that is treated may be wet age-related macular degeneration. Alternatively, in another embodiment, the retinal disorder that is treated may be a diabetic retinopathy, or any other retinal disorder associated with diabetes, such as diabetic macular oedema (DMO). In addition, the retinal disorder that is treated may be any pathophysiological condition which involves vascular leakage and a resultant damage to retinal structures.

In another embodiment, the constructs and vectors may be used to reduce vascular leakage activation and retinal cell damage. The constructs and vectors may be used to treat vascular leakage and retinal cell damage associated with the following conditions;

diabetic retinopathy, cancer, systemic capillary leak syndrome (SCLS)/Clarkson's syndrome, angioedema, severe trauma, shock, sepsis, multiple organ dysfunction syndrome (MODS), chronic kidney disease, end-stage renal disease, Kawasaki disease, severe Ebola virus disease, Dengue virus infection and/or mycobacterial infection.

It will be appreciated that the genetic construct according to the first aspect, or the recombinant vector according to the second aspect may be used in a medicament, which may be used as a monotherapy (i.e., use of the genetic construct according to the first aspect or the vector according to the second aspect of the invention), for treating, ameliorating, or preventing a retinal disorder, or for reducing vascular leakage and retinal cell damage. Alternatively, the genetic construct or the recombinant vector according to the invention may be used as an adjunct to, or in combination with, known therapies for treating, ameliorating, or preventing a retinal disorder, or for reducing vascular leakage and retinal cell damage.

An effective amount of recombinant viral vector is administered, depending on the objective of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells, in some embodiments at least about 20% of the desired tissue type, in some embodiments at least about 50%, in some embodiments at least about 80%, in some embodiments at least about 95%, in some embodiments at least about 99% of the cells of the desired tissue type. In some embodiments of the invention, the dose of viral particles administered to the subject is between 1×10⁸ to 1×10¹⁴ genome copies.

The recombinant virus particles may be administered by one or more injections, either during the same procedure or spaced apart by days, weeks, months or years. In some embodiments, multiple vectors may be used to treat the subject. In some embodiments, at least 2%, 5%, 10%, 15%, 20%, 25%, 30%, 35%, 40%, 45%, 50%, 55%, 60%, 65%, 70%, or 75% up to 100% of cells of the target tissue (for example retinal cells of the eye) are transduced. Methods to identify cells transduced by recombinant viral particles comprising a recombinant virus particle capsid are known in the art. For example, immunohistochemistry or the use of a market such as enhanced green fluorescent protein can be used to detect transduction of recombinant virus particles.

In some embodiments, the recombinant vectors are administered (for example by injection or infusion) to one or more locations in the desired tissue (for example the eye). In some embodiments, the recombinant vectors are administered (for example by injection or infusion) to any one of one, two, three, four, five, six, seven, eight, nine or ten, or more than ten locations in the tissue. In some embodiments, the recombinant vectors are administered to more than one location simultaneously or sequentially. In some embodiments, multiple injections of recombinant vectors are no more than one hour, two hours, three hours, four hours, five hours, six hours, nine hours, twelve hours or 24 hours apart.

The genetic construct or the recombinant vector according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The genetic construct or the recombinant vector according to the invention may also be incorporated within a slow or delayed-release device. Such devices may, for example, be inserted on or under the skin, and the medicament may be released over weeks or even months. The device may be located at least adjacent the treatment site. Such devices may be particularly advantageous when long-term treatment with the genetic construct or the recombinant vector is required and which would normally require frequent administration (e.g. at least daily injection).

In a preferred embodiment, medicaments according to the invention may be administered to a subject by injection into the blood stream, a nerve or directly into a site requiring treatment. For example, the medicament may be injected at least adjacent the retina. Injections may be intravitreal, suprachoroidal, subretinal, intraretinal, intravenous (bolus or infusion) or subcutaneous (bolus or infusion), or intradermal (bolus or infusion).

It will be appreciated that the amount of the genetic construct or the recombinant vector that is required is determined by its biological activity and bioavailability, which in turn depends on the mode of administration, the physiochemical properties of the genetic construct or the recombinant vector and whether it is being used as a monotherapy or in a combined therapy. The frequency of administration will also be influenced by the half-life of the transgene proteins within the subject being treated. Optimal dosages to be administered may be determined by those skilled in the art, and will vary with the particular genetic construct or the recombinant vector in use, the strength of the pharmaceutical composition, the mode of administration, and the advancement of the retinal oedema or the resulting damage to the retina or loss in retinal neurones. Additional factors depending on the particular subject being treated will result in a need to adjust dosages, including subject age, weight, gender, diet, and time of administration.

The genetic construct or the recombinant vector may be administered before, during or after onset of the retinal disorder. Daily doses may be given as a single administration (e.g. a single daily injection or inhalation of a nasal spray). Alternatively, the genetic construct or the recombinant vector may require administration twice or more times during a day. As an example, the genetic construct or the recombinant vector may be administered as two (or more depending upon the severity of the retinal disorder or retinal capillary dysfunction being treated) daily doses of between 0.001 μg/kg of body weight and 10 mg/kg of body weight of DNA plasmid, or between 1×10⁸ GC/mL and 1×10¹³ GC/mL of the viral vector (i.e. assuming a body weight of 70 kg). A patient receiving treatment may take a first dose upon waking and then a second dose in the evening (if on a two dose regime) or at 3 or 4-hourly intervals thereafter. Alternatively, a slow release device may be used to provide optimal doses of the genetic construct or the recombinant vector according to the invention to a patient without the need to administer repeated doses.

Known procedures, such as those conventionally employed by the pharmaceutical industry (e.g., in vivo experimentation, clinical trials, etc.), may be used to form specific formulations of the genetic construct or the recombinant vector according to the invention and precise therapeutic regimes (such as daily doses of the agents and the frequency of administration). The inventors believe that they are the first to suggest a bi-cistronic genetic construct encoding a promoter operably linked to coding sequences which will reduce VEGF concentrations below the level at which they produce pathophysiology, whilst simultaneously reducing or removing the subretinal fibrosis through CTGF neutralisation or attenuating complement activation.

According to a sixth aspect, there is provided a pharmaceutical composition comprising the genetic construct according to the first aspect, or the recombinant vector according to the second aspect, and a pharmaceutically acceptable vehicle.

According to a seventh aspect, there is provided a method of preparing the pharmaceutical composition according to the sixth aspect, the method comprising contacting the genetic construct according to the first aspect, or the recombinant vector according to the second aspect, with a pharmaceutically acceptable vehicle.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the genetic construct, the recombinant vector or the pharmaceutical composition is any amount which, when administered to a subject, is the amount of the aforementioned that is needed to treat dry age-related macular oedema or GA.

For example, the therapeutically effective amount of the genetic construct, the recombinant vector or the pharmaceutical composition used may be from about 1×10⁸ vector particles to about 1×10¹⁵ vector particles, and preferably from about 1×10¹¹ vector particles to about 1×10¹² vector particles.

In some embodiments, the viral titre of a pharmaceutical composition of the invention is between 5×10¹⁰, and 5×10¹³ genome copies per millilitre.

In some embodiments, the viral titre of a pharmaceutical composition of the invention is between 5×10¹⁰, and 5×10¹³ transducing units per millilitre. The term “transducing unit” as used in reference to a viral titre, refers to the number of infectious recombinant vector particles that result in the production of a functional transgene product as measured in functional assays, such as described in [57, 58].

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the genetic construct or recombinant vector according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a particle suspension in solution. Liquid vehicles are used in preparing solutions, suspensions, emulsions, syrups, elixirs and pressurized compositions. The genetic construct or the recombinant vector according to the invention may be dissolved or suspended in a pharmaceutically acceptable liquid vehicle such as water, ionic buffered solution, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intravitreal, suprachoroidal, subretinal, intraretinal, intracameral, intramuscular, intrathecal, epidural, intraperitoneal, intravenous and particularly subcutaneous injection. The genetic construct or the recombinant vector may be prepared as a sterile solid composition that may be dissolved or suspended at the time of administration using sterile water, saline, or other appropriate sterile injectable medium.

Pharmaceutically acceptable carriers, excipients, and diluents are relatively inert substances that facilitate administration or a pharmaceutically effective substance and can be supplied as a liquid solutions or suspensions, as emulsions, or as solid forms suitable for dissolution or suspension in liquid prior to use. For example, an excipient can give forms suitable for or consistency, or act as a diluent. Suitable excipients include, but are not limited to stabilizing agents, wetting and emulsifying agents, salts for varying osmolarity, encapsulating agents, pH buffering substances, and buffers. Such excipients include any pharmaceutical agent suitable for direct delivery to the subject (for example intravitreally or sub-retinally) which may be administered without undue toxicity. Pharmaceutical acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates and the like; and the salts of organic acids such as acetates, propionates, malonates, or benzoates.

In some embodiments, pharmaceutical acceptable excipients may include pharmaceutical acceptable carriers. Such pharmaceutically acceptable carriers can be sterile liquids, such as water and oil, including those of petroleum, animal, vegetable or synthetic origin, such as peanut oil, soybean oil, mineral oil. Saline solutions and aqueous dextrose, polyethylene glycol (PEG) and glycerol solutions can also be employed as liquid carriers, particularly for injectable solutions. Additional ingredients may also be used, for example preservatives, buffers, tonicity agents, antioxidants and stabilizers, non-ionic wetting or clarifying agents, or viscosity-increasing agents. A thorough discussion of pharmaceutical acceptable excipients and carriers is available in Remington's Pharmaceutical Sciences (Ed Remington J P and Gennaro A R; Mack Pub. Co. Easton, Pa 1990).

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No: 1-107, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW [59, 60] is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids. All of the features described herein (including any accompanying claims, abstract and drawings), and/or all of the steps of any method or process so disclosed, may be combined with any of the above aspects in any combination, except combinations where at least some of such features and/or steps are mutually exclusive.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figure, in which:

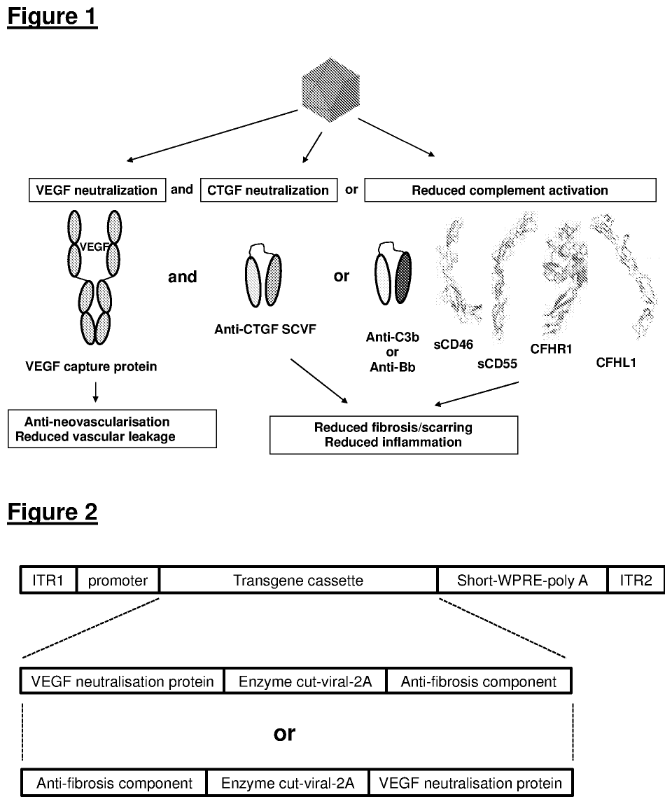

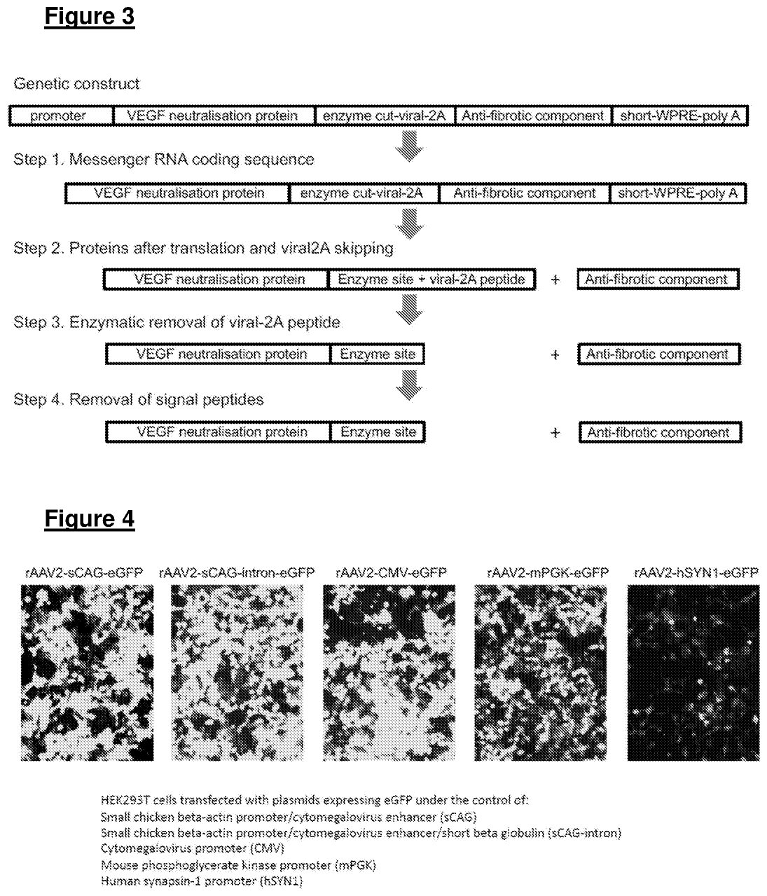

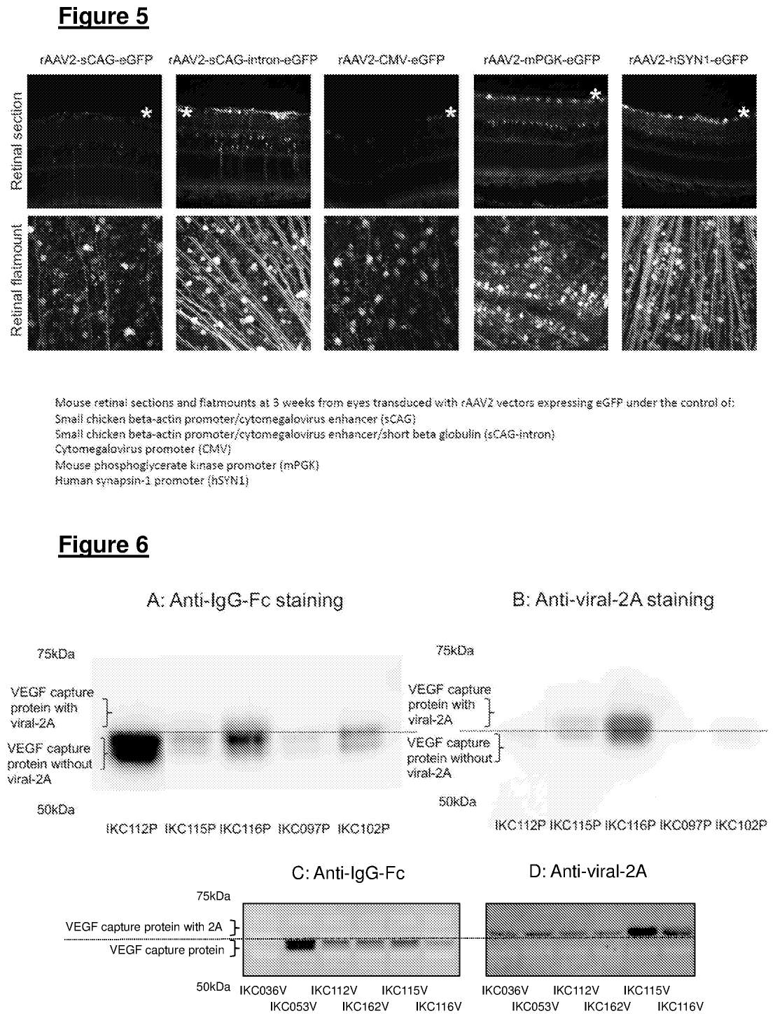

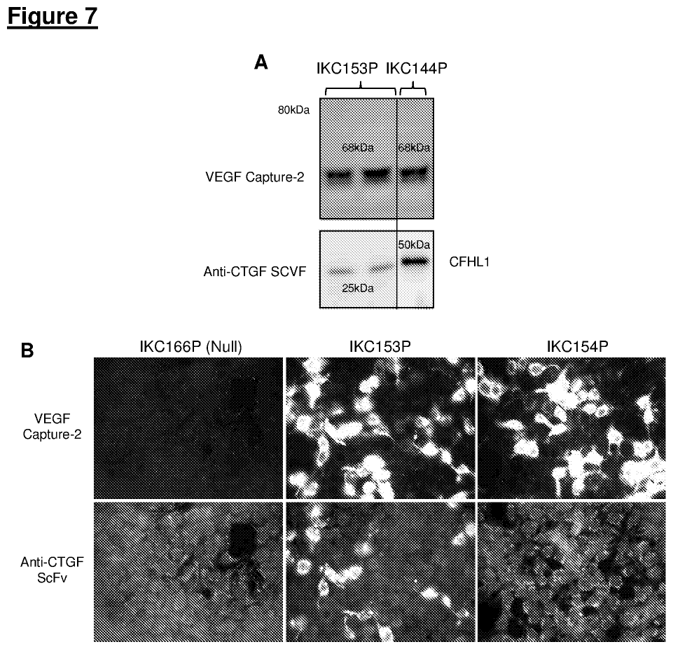

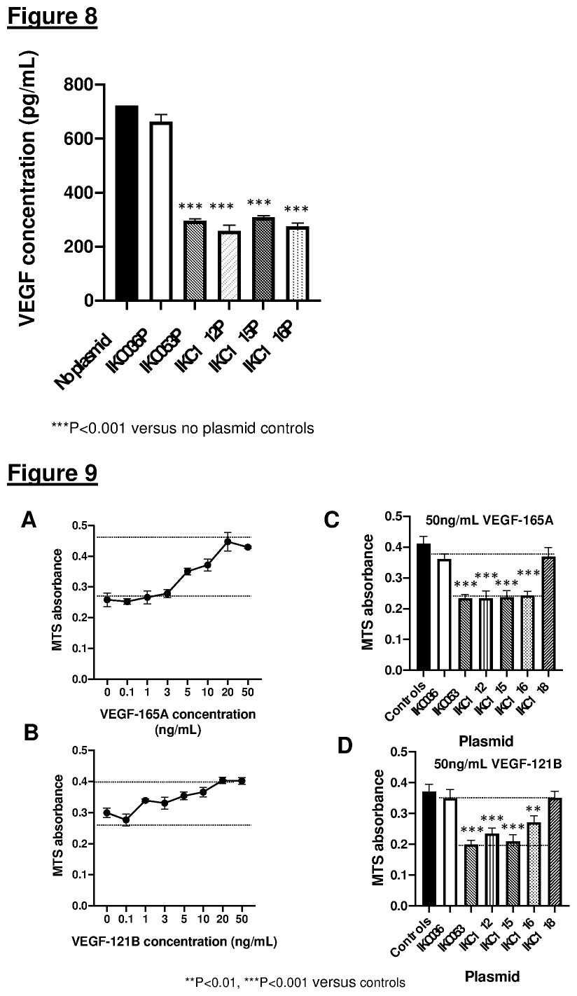

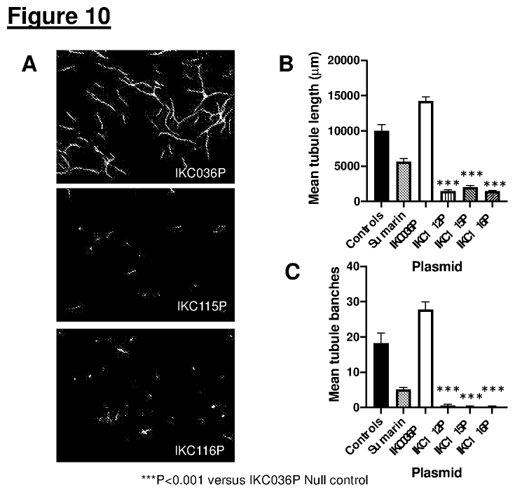

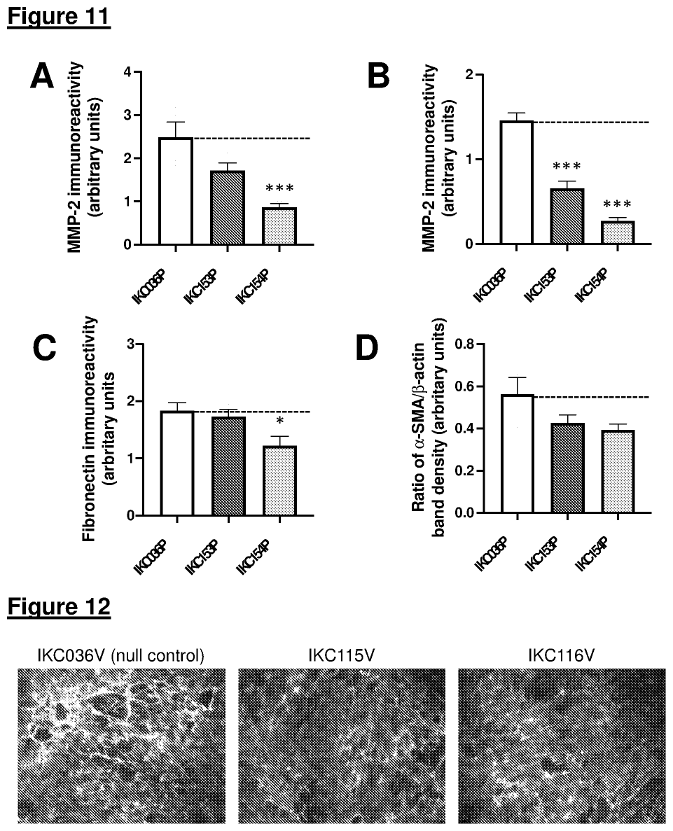

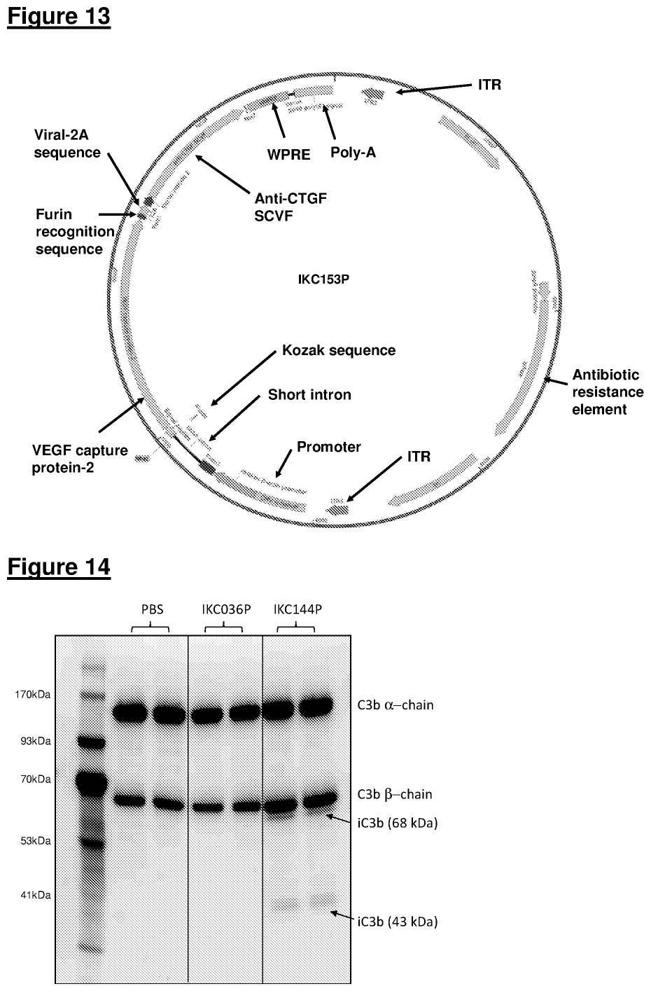

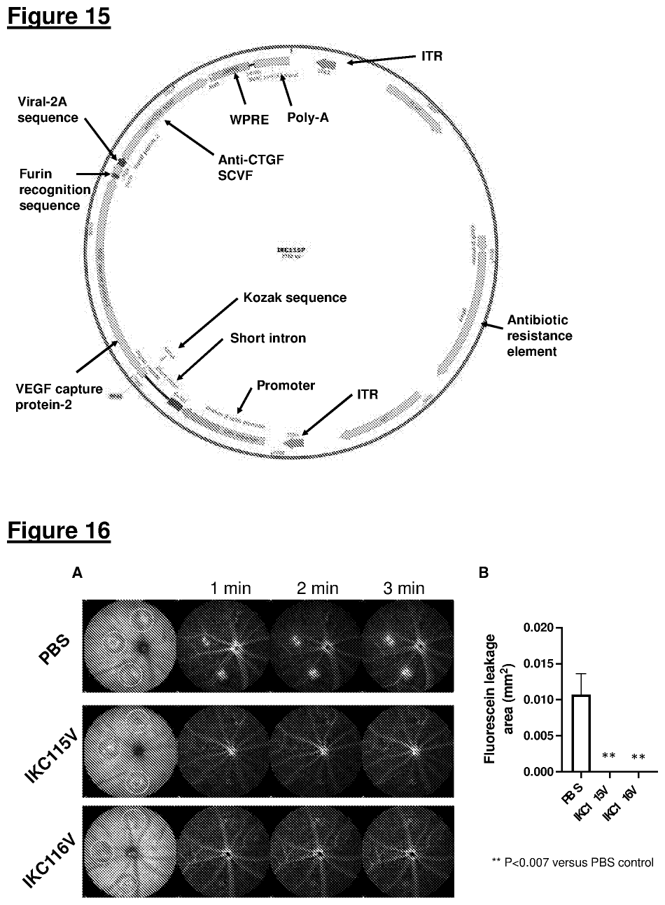

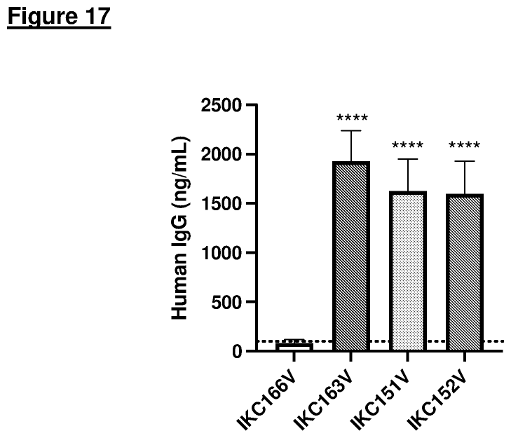

FIG. 1 is an illustration of one embodiment of a gene therapy viral vector (top of figure) according to the invention, which expresses various transgene proteins, i.e. an anti-VEGF protein and an anti-fibrotic protein, and their biological effects in reducing the pathophysiology associated with retinal disorders, such as wet-AMD. In the Figure, the anti-fibrotic protein is shown as being either an anti-CTGF (connective tissue growth factor) protein or an anti-complement protein.

FIG. 2 is a schematic drawing of one embodiment of a genetic construct according to the invention. The transgene cassette essentially encodes a single mRNA transcript from which two independently secreted proteins will be produced, the VEGF neutralisation component and the anti-fibrotic component. These components can be in either position (orientation), linked via the enzyme target/viral 2A cleavage/skipping site.

FIG. 3 illustrates the intracellular biochemical processing of the bi-cistronic construct from a single mRNA transcript into two mature therapeutic proteins. Step 1 is the transcription of the messenger RNA via the single promoter, followed by translation of the single large coding sequence. Step 2 is the translational skipping by the ribosome directed by the viral 2A sequences to give rise to two separate pro-proteins. Step 3 occurs at the level of the Golgi, in which the viral-2A sequences are cleaved from the pro-proteins via the activity of the furin/enzyme at the upstream cleavage site. Step 4 is the removal of the secretory protein signal peptides, which removes the remaining proline amino acid from the N-terminus of the downstream component, prior to secretion from the target retinal cells.

FIG. 4 shows images of enhanced Green Fluorescent Protein (eGFP) reporter gene expression in HEK293 cells taken 24 hours after transduction with rAAV2 vectors containing different promoter sequences: the small chicken beta-actin promoter/cytomegalovirus enhancer promoter (sCAG), the sCAG promoter followed by the addition of an intron created from fusing a short stretch of nucleotides derived from the 5′ and 3′ of rabbit beta-globulin intron (sCAG-intron), the cytomegalovirus promoter (CMV), the murine phosphoglycerate kinase promoter (mPGK) and the human synaptin-1 promoter (hSYN1).

FIG. 5 shows both cross-sectional and flatmount images of the mouse retina, to illustrate the level of eGFP expression three weeks after intravitreal injection with rAAV2 vectors containing different promoters: the small chicken beta-actin promoter/cytomegalovirus enhancer promoter (sCAG); the cytomegalovirus enhancer element plus the chicken beta-actin promoter and a short stretch of nucleotides derived from the 5′ and 3′ of rabbit beta-globulin intron (sCAG-intron); the cytomegalovirus promoter (CMV); the murine phosphoglycerate kinase-1 promoter (mPGK); and the human synaptin-1 (hSYN1) promoter. The symbol ‘*’ indicates the ganglion cell layer.

FIG. 6 shows Western blots illustrating the expression of VEGF capture-2 protein in the supernatant harvested from HEK293T cells 24 hours after transfection with a series of expression plasmids (A and B) or transduction with a series of rAAV2/2 vectors (C and D). FIGS. 6A and 6C shows the results of supernatant probed with an IgG-Fc antibody that detects the secreted VEGF capture-2 protein. FIGS. 6B and D shows the results of supernatant probed with an antibody that detects the viral-2A protein. The VEGF capture-2 protein with the viral-2A amino acid sequence still attached is shown above the dotted line and it is present in some of the lanes.

FIG. 7A shows Western blots illustrating the intracellular processing of the expression cassettes from IKC153P and IKC144P plasmid transfected HEK293T cells to secrete the VEGF capture-2 protein and anti-fibrotic component anti-CTGF SCVF-1 or CFHL1 respectively through translational skipping over the viral-2A sequence and subsequent removal via the furin enzymatic cleavage. FIG. 7B shows immunocytochemistry on HEK293T cells transfected with either the control (IKC166P) or the IKC154P (VEGFCap-2-furin-P2A-anti-CTGF SCVF-2) to show the detection of the VEGF Capture-2 protein and the anti-CTGF SCVF. Examples of the anti-fibrotic components are the anti-CTGF SCVF-1 and anti-CTGF SCVF-2 (IKC153P and IKC154P), and the CFHL1 protein (IKC144P).

FIG. 8 illustrates VEGF-165 concentrations measured by ELISA in cell culture medium generated by HEK293T cells 24 hours after transfection with a series of plasmids. The secreted proteins capable of VEGF-165 neutralisation were compared to medium from cells which did not receive any plasmid (No plasmid), or from cells which were transfected with IKC036P (Null-control plasmid). IKC112P comprises the novel VEGF-capture protein-2 only, IKC115P comprises VEGF capture protein-2-furin-viral P2A-anti-CTGF-SCVF-1, and IKC116P comprises VEGF capture protein-2-furin-viral P2A-anti-CTGF-SCVF-2.

FIG. 9 demonstrates the ability of VEGF-165A (FIG. 9A) or VEGF-121B (FIG. 9B) to induce a proliferation of human umbilical vascular endothelial cells (HUVECs) as measured by increased MTS absorbance. Addition of culture medium from HEK293T cells which have previously been transfected with various plasmid constructs shows that the transgene proteins released from the HRK293T cells transfected with the IKC053P, IKC112P, IKC115P and IKC116P plasmids are able to prevent the exogenous VEGF-165A or VEGF-121B-induced HUVEC cell proliferation compared to control untransfected HEK293T cells or HEK293T cells transfected with the Null control plasmid (IKC036P) or a plasmid expressing the anti-CTGF SCVF-1 only (IKC118P). The mechanism for HUVEC anti-proliferative efficacy is through the production and release of VEGF neutralising proteins from the IKC053P, IKC112P, IKC115P and IKC116P transfected HEK293T cells.

FIG. 10 further illustrates the ability of the plasmid constructs to produce proteins capable of preventing HUVEC proliferation when grown on a bed (layer) of human fibroblasts (HUVEC-fibroblast co-culture). FIG. 10A shows that the HUVECs express eGFP marker protein and the signal to noise has been improved by staining with an anti-eGFP antibody. Longer tubules are formed in the IKC036P control group compared to the IKC115P and IKC116P exposed group that both express the anti-VEGF transgene protein. FIG. 10B and FIG. 10C show the formation of longer HUVEC tubules with branching (under the influence) in the presence of 10 ng/ml of VEGF-165A (control and IKC036P null control) that is significantly inhibited by addition of culture medium from HEK293T cells transfected with plasmids and which generate VEGF neutralising proteins (IKC112P, IKC115P and IKC116P).

FIG. 11 is the quantification of Western blots to compare the level of fibrotic markers in ARPE-19 cells following transfection with IKC153P (precursor is IKC115P) or IKC154P (precursor is IKC116P) versus IKC036P Null plasmid. FIG. 11A shows the level of the pro-fibrotic enzyme MMP2 secreted from the cells under serum starved conditions. FIG. 11B shows the level of MMP2 secreted from the cells following stimulation by transforming growth factor-beta2 (TGF-β2). FIG. 11C shows the secreted level of fibronectin in the culture medium and FIG. 11D shows the level of α-smooth muscle actin (α-SMA) within the cells (cell lysate) normalised to β-actin.

FIG. 12 are confluent and serum starved ARPE-19 monolayers following transduction with the IKC036V (Null control), IKC115V or IKC116V and stained for fibronectin protein (grey). Note reduced fibronectin scaffold deposited in the IKC115V and IKC116V treated cells versus IKC036V Null control.

FIG. 13 shows an embodiment of a plasmid map (IKC153P) of the vector of the invention.

FIG. 14 is a C3b cleavage assay to demonstrate the activity of the CFHL1 component in the IKC144P plasmid. HEK293T cells were transfected with either the Null control plasmid (IKC036P) or the CFHL1 bi-cistronic plasmid (IKC144P) plasmid, untransfected HEK293T cells were used as an additional control (PBS). Following transfection, the HEK293T supernatant was collected and incubated with recombinant C3b and recombinant CFI proteins and then run on a Western blot and probed with a C3 antibody. FIG. 14 shows that C3b cleavage only occurred in the presence of the IKC144P plasmid but not in the presence of either of the controls (IKC036P or PBS only).

FIG. 15 shows an embodiment of a plasmid map (IKC115P) of the vector of the invention.

FIG. 16 shows the results from a mouse lasered-induced choroidal neovascularisation (CNV) study in which mice were intravitreally treated with PBS control or the IKC115V or IKC116V vectors. FIG. 16A shows serial images at the choroidal focal plane at 1, 2 and 3 minutes after fluorescein administration to demonstrate the level of leakage from each lasered site (circled). FIG. 16B is a graph to show the fluorescein leakage area on day 14 post-CNV, showing that the IKC115V and the IKC116V treatment groups had significantly lower CNV leakage area as compared to the PBS treatment group.

FIG. 17 illustrates that intravitreal injection of either the rAAV2 IKC151V or the IKC152V bi-cistronic vectors in the mouse significantly increases the vitreal concentration of the VEGF-capture-2 protein compared to the IKC166V null vector treated mice, as measured by IgG ELISA on samples taken at 3 weeks post vector administration (****P<0.0001; one-way ANOVA). In addition, the vitreous concentrations of VEGF-Capture-2 produced by the bi-cistronic vectors IKC151V and IKC152V were similar to that generated by the mono-cistronic vector IKC163V which produces only aflibercept. Importantly, the level of expressed VEGF-Capture-2 protein is above the baseline of the modelled aflibercept pharmacokinetic range post single aflibercept (2 mg) dose in patients measured 6 weeks after bolus injection [61]. This study demonstrates that inclusion of a secondary coding component that attenuates the development and potentially reverses subretinal fibrosis does not reduce the capacity of the vector to neutralise the common isoforms of soluble VEGF.

### EXAMPLES

Referring to FIGS. 1 and 2, the inventors have designed and constructed a novel genetic construct, which encodes (i) an anti-VEGF protein and (ii) an anti-fibrotic protein (either an anti-complement protein or an anti-CTGF protein, such as an antibody, or antigen-binding fragment thereof), under the control of a single promoter. As illustrated in FIG. 2, the inventors also introduced a spacer sequence into the genetic construct (e.g. a viral-2A peptide spacer sequence), which advantageously enables expression of all of the peptides encoded by the construct to occur under control of a single promoter, as a single mRNA transcript. Additionally, in order to enzymatically remove the viral-2A peptide sequence from the C-terminal of the proteins, the inventors introduced a viral-2A removal sequence into the construct, such as a furin recognition sequence.

As illustrated in FIG. 3, the bi-cistronic expression cassette produces two mature therapeutic proteins, an anti-VEGF protein and an anti-fibrotic protein. The anti-VEGF protein acts to prevent neovascularisation and reduce vascular leakage. The anti-fibrotic protein reduces fibrosis, scarring and inflammation.

The inventors then introduced the genetic construct into recombinant expression vectors, such as rAAV2 (for example, see FIGS. 13 and 15).

### Materials and Methods

### DNA Plasmid Design and Production

Codon optimisation of DNA sequences was performed using the tools (http://www.jcat.de) or the Genscript online tool. Synthetic DNA blocks and cloning were performed using standard molecular biology techniques. All DNA Plasmids were scaled up in SURE competent cells (Agilent Technologies) overnight following maxi-prep purification with minimal endotoxin presence.

IKC036P is a Null control, IKC053P is a reference plasmid designed to produce and secrete the VEGF capture protein-1 only. IKC112P comprises the novel VEGF-capture protein-2 only, IKC115P comprises VEGF capture protein-2-furin-viral P2A-anti-CTGF-SCVF-1, IKC116P comprises VEGF capture protein-2-furin-viral P2A-anti-CTGF-SCVF-2, IKC097P comprises VEGF capture protein-2-furin-viral P2A-anti-CTGF-SCVF (non-optimised), IKC102P comprises VEGF capture protein-2-furin-viral P2A-anti-CTGF-SCVF-1 (non-optimised), and IKC118P comprises anti-CTGF-SCVF-1.

### Recombinant AAV Vector Production

Recombinant AAV2 vectors were manufactured using the DNA plasmids. HEK293T cells (2.5×10⁸) were transduced with a total of 500 μg of the three plasmids (Rep-2-Cap2, pHelper and ORF and ITR containing plasmid). Following freeze-thaw of the HEK293T cells to liberate the viral vector particles, followed by iodixanol gradient ultracentrifugation and de-salting. The vectors were suspended in Dulbecco's phosphate-buffered saline (DPBS) buffer from Thermo Fisher/Gibco manufactured to cGMP standard (cat number 14190250 consisting of 8 g/L NaCl, 1.15 g/L of Na₂HPO₄, 0.2 g/L of KCl and 0.2 g/L of K₂HPO₄ with no calcium or magnesium; pH 7.0-7.3, 270-300 mOsm/kg) the following vector titres were obtained by qPCR using primers recognising the ITR region.

FIG. 4 illustrates enhanced Green Fluorescent Protein (eGFP) reporter gene expression in HEK293T cells taken 24 hours after plasmid transfection with constructs containing different promoter sequences: sCAG, CMV, hSYN1, mPGK, and sCAG-intron. As illustrated in FIG. 4, both sCAG and CMV display high level of eGFP transgene expression in HEK293T cells.

FIG. 5 shows both cross-sectional and flatmount images of the mouse retina to illustrate the level of eGFP expression three weeks after intravitreal injection with 5×10\hat{ }9 GC/eye rAAV2 vectors containing different promoter sequences: sCAG, CMV, hSYN1, mPGK and sCAG-intron. As can be seen from FIG. 5, the addition of the intron in the sCAG-intron promoter increases retinal expression compared to the same promoter without the intron, i.e. sCAG.

### Example 1—Detection of Furin Activity and Viral-2A Peptide Cleavage

Briefly, DNA plasmids were transfected in to cells by mixing the plasmid with Opti-MEM (FisherSci) and lipofectamine 3000 (FisherSci). HEK293T cells were optimally transfected at 80% confluency in 6-well plates such that each well received 2 μg of plasmid DNA and 3.75 μL lipofectamine. Cells were incubated for 24 hours at 37° C., 5% CO₂.

Western blotting of the harvested supernatants was used to visualise separation of secreted VEGF capture-2 protein from the anti-CTGF/complement protein. VEGF capture proteins which contain either the wild-type or a modification of the human IgG-Fc portion were detected using HRP-conjugated anti-human IgG Fc antibody (goat anti-IgG Fc (HRP conjugated, ab98624; Abcam, diluted at 1:7000). The antibody (NBP2-59627; NovusBio, 1:1000;) was used to examine viral-2A peptide removal from the C-terminals of first transgene proteins.

HEK293T cells were transfected with plasmids as described above. To confirm cleavage of the viral-2A peptide from the C-terminal, an IgG-Fc antibody was used to detect the secreted VEGF capture-2 protein in all supernatants evaluated. As illustrated in FIGS. 6A and 6C, the majority of VEGF capture-2 protein is cleaved at the furin site and is detected without the presence of the viral-2A. Importantly, the cleaved VEGF capture-2 protein runs at the same size as the VEGF capture-2 protein derived from the IKC112P transfected cells, which does not include the viral-2A sequence.

Additionally, as shown in FIG. 6B, the cells were also probed with an antibody that detects the viral-2A protein. Cross-reactivity (non-specific staining) can be seen at the same molecular weight as the VEGF capture-2 protein (IKC036P and IKC036V Null vector lanes) and derived from the IKC112P transfected cells, which does not contain the viral 2A sequence. The viral 2A antibody also picks up (two) heavier molecular weight bands in the IKC115P and IKC116P supernatants (FIG. 6B), indicative that most but not all of the viral-2A sequence has been cleaved at the furin site. The bands are weaker in the IKC097P and IKC102P supernatants, which are earlier expression constructs, showing that the viral-2A sequence has been cleaved at the furin site.

Further Western blotting and immunocytochemistry (FIG. 7) confirmed the expression and separation of both transgenes from cells transfected with the bi-cistronic plasmid constructs. In FIG. 7A, HEK293T cells transfected with the IKC153P and IKC144P plasmids express the VEGF-Capture-2 protein (goat anti-IgG Fc (HRP conjugated, ab98624; Abcam, diluted 1:2000) and either the Ikarovec commissioned anti-CTGF SCVF-1 (rabbit polyclonal (peptide 1); GenScript, diluted 1:500) or the CFHL-1 (rabbit anti-CFH, ab133536; Abcam, diluted 1:1000) respectively. In FIG. 7B, the transfected cells were co-labelled with the above antibodies for IgG Fc (1:1000) or anti-CTGF SCVF-1 (1:1000). Note that the anti-CTGF SCVF-1 (rabbit polyclonal (peptide 1) did not immunolabel anti-CTGF SCVF-2 in plasmid IKC154P and no non-specific staining was detected in the Null control (IKC166P).

### Example 2—VEGF Concentration in HEK293T Cells after Transfection with Various Plasmid Constructs

HEK293T cells were transfected with plasmids as described above. The HEK293T cell incubation medium was collected and centrifuged to remove any cell debris and the VEGF-165 concentrations generated from the cells were subsequently measured using a commercial human VEGF ELISA kit (ab222510; Abcam). The results were compared to the medium from cells which were not transfected with a plasmid (no plasmid) and cells which were transfected with IKC036P (Null plasmid). VEGF-165 concentrations were measured and the results were compared to the medium (supernatant) from cells which were not transfected with a plasmid (no plasmid) and cells which were transfected with IKC036P (Null plasmid). As illustrated in FIG. 8, a significant reduction in VEGF concentration was observed in cells transfected with a plasmid comprising an anti-VEGF protein component.

### Example 3—Reduction of VEGF-Induced Human Umbilical Vascular Endothelial Cell (HUVEC) Growth with Plasmid Transgene Products

Medium from HEK293T cell incubation medium which had previously been transfected with no plasmid (control) or test plasmid DNA in Opti-MEM for 6 h, followed by exchange to serum-free DMEM, high glucose, GlutaMAX (FisherSci) for 24 h, was added to HUVECs grown in endothelial cell media (ECM, C22010; PromoCell) containing 1×Pen-Strep in a 96-well plate (3,000 cells/well). Recombinant additional VEGF (5-100 ng/ml; Caltag-Medsystems) was then added to each well and the HUVECs were incubated for 72 hours. Cell growth was measured by spectrophotometry using MTS (G3580, CellTitre 96® aqueous One solution; Promega). Absorbance was read at 490 nm.

As illustrated in FIG. 9, culture medium from HEK293T cells which had previously been transfected with plasmids, did not show HUVEC proliferation in the presence of 50 ng/mL VEGF-165A or VEGF-121B (FIGS. 9C and D). This demonstrates complete VEGF neutralisation with the anti-VEGF plasmid constructs.

### Example 4—HUVEC-Fibroblast Co-Culture and Generation of Vascular Network

The Caltag-Medsystems angiogenesis endothelial/fibroblast co-culture kit was used, which consists of eGFP-expressing HUVECs and human fibroblasts in a ratio of 1:30. Endothelial cells initially form small islands within the culture matrix and subsequently proliferate, eventually forming threadlike tubule structures in the gel matrix to form a network of anastomosing tubules within 10 days of culture. The angiogenesis co-culture is known to be responsive to known micro- and macro-molecular inhibitors and stimulators of angiogenesis. Medium from HEK293T cells which had previously been transfected with plasmids (using the protocol described above) was added to the co-cultures and supplemented with 10 μg/mL of recombinant human VEGF (ab9571; Abcam) every 2-3 days from Day 2. Control cultures received sumarin which blocks the effects of fibroblast growth factor and other growth factors generated by the fibroblasts. The tubular HUVEC structure was fixed and imaged at the end of the experiment for eGFP-expressing cells using fluorescence microscopy and additionally stained with anti-GFP antibodies (A11122; Invitrogen, diluted 1:2,000). Average tubular length and branching was calculated using MetaLabs FastTrack tubule formation AI program.

As illustrated in FIG. 10A, HUVEC expansion was significantly inhibited following exposure to medium from HEK293T cells transfected with expression plasmids IKC0115P and IKC116P compared to null control IKC036P. Additionally, as illustrated in FIGS. 10B and 10C, HUVEC tubule length and branching was significantly reduced following exposure to medium from HEK293T cells transfected with IKC115P and IKC116P when compared to the control.

### Example 5—Reduction in Fibrotic Marker Protein Expression in ARPE-19 Cells after Transfection with Various Plasmids

ARPE-19 cells were transfected with plasmids as described above in Opti-MEM for 24 h. On day 2, medium was exchanged with DMEM/F12+5% FBS and then to serum free DMEM/F12 on day 3. On day 5, TGF beta 2 (10 ng/ml, ab84070; Abcam) or fresh serum free DMEM/F12 was added to the confluent, transfected cells and supernatants and lysates collected on day 7. Western blotting of the supernatant from serum starved cells revealed a decrease in fibronectin (ab268020; Abcam, diluted 1:500) (FIG. 11A) and MMP2 (ab92536; Abcam diluted 1:1000) (FIG. 11C) with significance shown for IKC154P (precursor was known as IKC116P). A similar reduction in MMP2 was observed in supernatant from TGF beta 2 stimulated cells with significance shown for both IKC153P (precursor was known as IKC115P) and IKC154P (precursor was known as IKC116P) (FIG. 11B). Lysate samples revealed a slight decrease in alphaSMA (ab5694; Abcam, diluted 1:500) in the TGF beta 2 stimulated group with plasmids IKC153P (precursor was known as IKC115P) and IKC154P (precursor was known as IKC116P) compared to IKC036P (Null control) (FIG. 11D).

As illustrated in FIG. 12, a reduction in fibronectin (ab268020; Abcam, diluted 1:200) immunolabelling could be seen in ARPE-19's transduced with the vectors IKC115V and IKC116V compared to IKC037V (Null control) at 5 days post transduction.

### Example 6—C3b Cleavage Assay to Evaluate CFHL1

HEK293T cells were transfected with plasmids as described above. The HEK293T cell incubation medium was collected and centrifuged to remove any cell debris and then 50 μL of the fresh supernatant was incubated for 1 h at 37° C. with 42 nM recombinant C3b and 1.2 nM recombinant CFI. Following incubation, the samples were then evaluated by Western blotting using a C3 antibody (Goat-anti human-C3, AHP1752, 1:2000 and secondary Donkey anti-goat, 705-035-147, 1:10,000).

As illustrated in FIG. 14, C3b cleavage to the iC3b 68 and 43 kDa fragments occurred in the presence of the supernatant derived from the HEK293T cells transfected with the IKC144P plasmid that expresses the CFHL1 component but not in the presence of the control (IKC036P) or untransfected supernatants. These demonstrate that the CFHL1 component expressed by the bi-cistronic IKC144P plasmid is enzymatically active.

### Example 7—Efficacy of the Bi-Cistronic rAAV Vectors in the Mouse Laser CNV Model

Mice received unilateral intravitreal injections (2 μL) of rAAV vectors IKC115V (8.7×10⁹ genome copies (GC)/eye) or IKC116V (8.7×10⁹ GC/eye) or control PBS and 3 weeks later choroidal neovascularisation was induced in the treated eyes by laser photocoagulation using a 532 nm diode laser (spot size: 10 μm; power: 130 mW; time: 120 ms Oculight TX. Iridex Corp). Mouse eyes were imaged using fluorescein angiography (FA) 2 weeks post-lasering using a Heidelberg Spectralis HRA system (Heidelberg Engineering) and a solution of 2.5% sodium fluorescein (Sigma-Aldrich) that was administered as a subcutaneous injection (30 μL/10 g). Consecutive fluorescent images (Sensitivity: 45; ART mean: 5 frames) were taken every 60 seconds from the choroidal focus level for a period of 5 minutes after fluorescein administration.

As illustrated in FIG. 16, intravitreal administration of the bi-cistronic rAAVs, IKC115V and IKC116V, significantly decreased CNV leakage area detected from FA images two weeks post-CNV as compared to the PBS control treated mice.

### Example 8—Detection of Clinically Relevant Levels of VEGFCapture-2 Expressed from the Bi-Cistronic rAAV Vectors in Vivo

The concentration of VEGF Capture-2 protein in the mouse vitreous following intravitreal delivery of the IKC151V and IKC152V rAAV vectors is shown in FIG. 17, compared to the concentration of aflibercept protein (expressed from IKC163V).

Mice were injected intravitreally (2 μL) with IKC151V (2.3×10¹⁰ GC/eye), IKC152V (2.7×10¹⁰ GC/eye), IKC163V (7.8×10⁹ GC/eye) or IKC166V (2.1×10¹⁰ GC/eye. After 21 days their eyes were dissected free and vitreous sample (between 4-5 μL) was extracted. Concentration of the VEGF Capture-2 or aflibercept protein within the vitreous was measured using a commercial IgG ELISA kit (Abcam). Of note, the concentration of VEGF Capture-2 in the vitreous at 3 weeks post injection in the bi-cistronic (IKC151V and IKC152V) treated eyes is above a predicted therapeutically relevant clinical level of aflibercept based on the data at 4 and 6 weeks of a 2 mg bolus of Aflibercept in patients, represented in FIG. 17 by the dotted line. This demonstrates that inclusion of a secondary coding component that attenuates the development and potentially reverses subretinal fibrosis, does not reduce the capacity of the vector to neutralise the common isoforms of soluble VEGF.

### Discussions and Conclusions

As illustrated in the Examples, the inventors have demonstrated that it is surprisingly possible to combine the open reading frames (ORFs) which code for the anti-VEGF protein and the anti-fibrotic protein, in a single genetic construct. This was especially challenging given the large sizes of the genes, and it could not have been predicted that it would have been possible to co-express these components in physiologically useful concentrations from a single expression cassette, and for that expression cassette to be accommodated by a rAAV-2 vector.

As demonstrated in the Examples, the anti-VEGF protein of the claimed construct and vector is able to reduce VEGF concentrations, thereby preventing neovascularisation and reducing vascular leakage. Additionally, the presence of the anti-fibrotic protein reduces fibrosis, scarring and inflammation. Advantageously, with the construct of the invention, there is no need to inject a recombinant protein as described in the prior art, and the construct only requires a single administration to achieve long-term therapeutic effect.

### REFERENCES

1. Mehta S (2015). Age-Related Macular Degeneration. Prim. Care, vol. 42(3), PP: 377-391.
2. Cheung L K, Eaton A (2013). Age-related macular degeneration. Pharmacotherapy, vol. 33(8), PP: 838-855.
3. Lim L S, Mitchell P, Seddon J M, Holz F G, Wong T Y (2012). Age-related macular degeneration. Lancet, vol. 379(9827), PP: 1728-1738.
4. Congdon N, O'Colmain B, Klaver C C, Klein R, Muñoz B, Friedman D S, Kempen J, Taylor H R, Mitchell P, Eve Diseases Prevalence Research Group (2004). Causes and prevalence of visual impairment among adults in the United States. Arch Ophthalmol., vol. 122, PP: 477-485.
5. Lotery A, Griner R, Ferreira A, Milnes F, Dugel P (2017). Prevalence of age-related macular degeneration in an elderly UK Caucasian population—The Bridlington Eye Assessment Project: a cross-sectional study. Eye (Lond). Vol. 31(12), PP: 1697-1706.
6. Global burden of disease study 2013 collaborators (2015). Global, regional, and national incidence, prevalence, and years lived with disability for 301 acute and chronic diseases and injuries in 188 countries, 1990-2013: a systematic analysis for the Global Burden of Disease Study 2013. Lancet, vol. 386(9995), PP: 743-800.
7. GBD 2015 disease and injury incidence and prevalence collaborators (2016). Global, regional, and national incidence, prevalence, and years lived with disability for 310 diseases and injuries, 1990-2015: a systematic analysis for the Global Burden of Disease Study 2015. Lancet, vol. 8(388), PP: 1545-1602.
8. Wong W L, Su X, Li X, Cheung C M, Klein R, Cheng C Y, Wong T Y (2014). Global prevalence of age-related macular degeneration and disease burden projection for 2020 and 2040: a systemic review and meta-analysis. Lancet Glob. Health, vol. 2(2), e106-e116.
9. Brown M M, Brown G C, Stein J D, Roth Z, Campanella J, Beauchamp G R (2205). Age-related macular degeneration: economic burden and value-based medicine analysis. Can. J. Ophthalmol., vol. 40(3), PP: 277-287.
10. Cunningham J (2017). Recognizing age-related macular degeneration in primary care. JAAPA., vol. 30, PP: 18-22.
11. Hernández-Zimbrón L F, Zamora-Alvarado R, Ochoa-De la Paz L, Velez-Montoya R, Zenteno E, Gulias-Cañizo R, Quiroz-Mercado H, Gonzalez-Salinas R (2018). Age-Related Macular Degeneration: New Paradigms for Treatment and Management of AMD. Oxid. Med. Cell Longev., e8374647.
12. Thorton J, Edwards R, Mitchell P Harrison R A, Buchan I, Kelly S P (2005). Smoking and age-related macular degeneration: a review of association. Eye (Lond.), vol. 19(9), PP: 935-944.
13. Evans J R, Lawrenson J G (2017). Antioxidant vitamin and mineral supplements for preventing age-related macular degeneration. Cochrane Database Syst. Rev., vol. 7, CD000253.
14. Shibuya M (2011). Vascular endothelial growth factor (VEGF) and its receptor (VEGFR) signaling in angiogenesis: a crucial target for anti- and pro-angiogenic therapies. Genes and Cancer vol. 2(12), PP: 1097-1105.
15. Apte R S, Chen D S, Ferrara N (2019) VEGF in signaling and disease: beyond discovery and development. Cell, vol. 176, PP: 1248-121264.
16. Schmidt-Erfurth U, Chong V, Loewenstein A, Larsen M, Souied E, Schlingemann R, Eldem B, Monés J, Richard G, Bandello F (2014). Guidelines for the management of neovascular age-related macular degeneration by the European Society of Retina Specialists (EURETINA). Brit. J. Ophthamol., vol. 98(9), PP: 1144-1167.
17. Sophie R, Akhtar A, Sepah Y. J, Ibrahim M, Bittencourt M, Do D V, Nguyen Q D (2012). Aflibercept: A potent vascular endothelial growth factor antagonist for neovascular age-related macular degeneration and other retinal vascular diseases. Biol. Ther., vol. 2(1), PP: 3.
18. Rosefield P. J, Brown D M, Heier J S, Boyer D S, Kaiser P K, Chung C Y, and Kim R Y, MARINA Study Group (2006). Ranibizumab for neovascular age-related macular degeneration. New Engl. J. Med., vol. 355, PP: 1419-1431.
19. Lotery A, Griner R, Ferreira A, Milnes F, Dugel P (2017). Real-world visual acuity outcomes between ranibizumab and aflibercept in treatment of neovascular AMD in a large US data set. Eye (Lond.) vol. 31(12), PP: 1697-1706.
20. Yang S, Zhao J, Sun X (2016). Resistance to anti-VEGF therapy in neovascular age-related macular degeneration: a comprehensive review. Drug Design, Dev. And Ther., vol. 10, PP: 1857-1867.
21. Daniel E, Toth C A, Grunwald J E, Jaffe G J, Martin D F, Fine S L, Huang J, Ying G S, Hagstrom S A, Winter K, Maguire M G, Comparison of Age-related Macular Degeneration Treatments Trials Research Group (2014). Risk of scar in the comparison of age-related macular degeneration treatments trials. Ophthamol., vol. 121(3), PP: 656-666.
22. Ishikawa K, Kannan R, Hinton D R (2016) Molecular mechanisms of subretinal fibrosis in age-related macular degeneration. Exp. Eye Res., vol. 142, PP: 19-25.
23. Bradham D M, Igarashi A, Potter R L, Grotendorst G R (1991). Connective tissue growth factor: a cysteine-rich mitogen secreted by human vascular endothelial cells is related to the SRC-induced immediate early gene product CEF-10, J. Cell Biol. vol. 114(6), PP: 1285-1294.
24. DeWinter P, Leoni P, Abraham D (2008). Connective tissue growth factor: Structure-function relationship of a mosaic, multifunctional protein. Growth Factors, vol. 26, PP: 80-91.
25. Holbourn K P, Acharya K R, Perbal B (2008). The CCN family of proteins: structure-function relationships. Trendds Biochem. Sci. vol. 33(10), PP: 461-473.
26. Igarashi A, Okochi H, Bradham D M, Grotendorst G R (1993). Regulation of connective tissue growth factor gene expression in human skin fibroblasts and during wound repair. Mol. Biol. Cell., vol. 4(6), PP: 637-645.
27. Lipson K E, Wong C, Teng Y, Spong S (2012). CTGF is a central mediator of tissue remodelling and fibrosis and its inhibition can reverse the process of fibrosis. Fibrogenesis & Tissue Repair, vol. 5(Suppl 1), S24.
28. Tamatani T, Kobayashi K, Tesuka K, Sakamoto S, Suzuki K, Nakanishi T, Takigawa M, Miyano T (1998). Establishment of the enzyme-linked immunosorbent assay for connective tissue growth factor (CTGF) and its detection in the sera of biliary atresia. Biochem. Biophys. Res. Commun., vol. 251(3), PP: 748-752.
29. Wunderlich K, Pech M, Eberle A N, Mihatsch M, Flammer J, Meyer P (2000). Expression of connective tissue growth factor (CTGF) mRNA in plaques of human anterior subcapsular cataracts and membranes of posterior capsule opacification Curr. Eve Res. vol. 21(2), PP: 627-636.
30. Winkler J L, Kedees M H, Guz Y, Teitelman G (2012). Inhibition of connective tissue growth factor by small interfering ribonucleic acid prevents increase in extracellular matrix molecules in a rodent model of diabetic retinopathy. Mol. Vis., vol. 18, PP: 874-886.
31. Blalock T D, Gibson D. J, Duncan M R, Tuli S S, Grotendorst G R, Schultz G S (2012). A connective tissue growth factor signalling receptor in corneal fibroblasts. Invest. Ophthamol. Vis. Sci., vol. 53(7), PP: 3387-3394.
32. Aoyama E, Kubota S, Takigawa M (2012). CCN2/CTGF binds to fibroblast growth factor receptor 2 and modulates its signalling. FEBS Letts., vol. 586, PP: 4270-4275.
33. Rayego-Mateos S, Rodrigues-Diez R, Morgado-Pascual J L, Rodrigues Diez R R, Mas S, Lavoz C, Alique M, Pato J, Keri G, Ortiz A, Egido J, Ruiz-Ortega M (2013). Connective tissue growth factor is a new ligand of epidermal growth factor receptor. J. Mol. Cell Biol., vol. 5, PP: 323-335.
34. Babic A M, Chen C-C, Lau L F (1999). Fisp12/Mouse Connective Tissue Growth Factor Mediates Endothelial Cell Adhesion and Migration through Integrin αᵥβ₃, Promotes Endothelial Cell Survival, and Induces Angiogenesis in Vivo. Mol. Cell Biol., vol 19(4), PP: 2958-2966.
35. Jedsadayanmata A, Chen C C, Kireeva M L, Lau L F, Lam S C-T (1999). Activation-dependent Adhesion of Human Platelets to Cyr61 and Fisp12/Mouse Connective Tissue Growth Factor Is Mediated through Integrin α_{IIb}β₃. J. Biol. Chem., vol. 274, PP: 24321-24327.
36. Schober J M, Chen N, Grzeszkiewicz T M, Jonanovic I, Emeson E E, Ugarova T P, Ye R D, Lau L F, Lam S C-T (2002). Identification of integrin α_{M}β₂ as an adhesion receptor on peripheral blood monocytes for Cyr61 (CCN1) and connective tissue growth factor (CCN2): immediate-early gene products expressed in atherosclerotic lesions. Blood, vol. 99(12), PP: 4457-4465.
37. Wahab N A, Weston B S, Mason R M (2005). Connective Tissue Growth Factor CCN2 Interacts with and Activates the Tyrosine Kinase Receptor TrkA. J. Am. Soc. Nephrol., vol. 16, PP: 340-351.
38. van Setten G, Berglin L, Blalock T D, Schultz G (2005). Detection of connective tissue growth factor in subretinal Fluid following retinal detachment: possible contribution to subretinal scar formation, preliminary results. Ophthalmic Res., vol. 37(6), PP: 289-292.
39. Kaasbøll J O, Gadicherla A K, Wang J-H, Monsen V T, Hagelin EMV, Dong M-Q, Attramadal H (2018). Connective tissue growth factor (CCN2) is a matricellular preproprotein controlled by proteolytic activation. J. Biol. Chem., vol. 293(16), PP: 17953-17970.
40. Reynolds R, Hartnett M E, Atkinson J P, Giclas P C, Rosner B, Seddon J M (2009). Plasma complement components and activation fragments: associations with age-related macular degeneration genotypes and phenotypes. Invest. Ophthamol. Vis. Sci., vol. 50(12), PP: 5818-5827.
41. Scholl H P N, Issa P C, Walier M, Janzer S, Poolok-Kopp B, Borncke F, Fritsche L G, Chong N V, Fimmers R, Wienker T, Holz F G, Weber B H F, Oppermann M (2008). Systemic complement activation in age-related macular degeneration. PLOS One, vol. 3(7), e2593.
42. Lechner J, Chen M, Hogg R E, Toth L, Silvestri G, Chakravarthy U, Xu H (2016). Higher plasma levels of complement C3a, C4a and C5a increase the risk of subretinal fibrosis in neovascular age-related macular degeneration: Complement activation in AMD. Immun. Ageing, vol. 13(4), s12979-016-0060-5.
43. Trou L A, Pickering M C, Blom A M (2017). The complement system as a potential therapeutic target in rheumatic disease. Nature Rev., vol. 13, PP: 538-547.
44. Anderson D H, Radeke M J, Gallo N B, Chapin E A, Johnson P T, Curletti C R, Hancox L S, Hu J, Ebright J N, Malek G, Hauser M A, Rickman C B, Bok D, Hageman G S, Johnson L V (2010). The pivotal role of the complement system in aging and age-related macular degeneration: hypothesis re-visited. Prog. Retin. Eye Res., vol. 29, PP: 95-112.
45. Scholl H P N, Issa P C, Walier M, Janzer S, Pollok-Kopp B, Borncke F, Fritsche L G, Chong N V, Fimmers R, Wienker T, Holz F G, Weber B H, Oppermann M (2008). Systemic complement activation in age related macular degeneration. PLOS ONE, vol. 3(7) P: e2593.
46. Nilsson S C, Sim R B, Lea S M, Fremeaux-Bacchi V, Blom Am (2011) Complement factor I in health and disease. Mol. Immunol. Vol., 48(14), PP: 1611-1620.
47. Lachmann P J (2019) The story of complement factor I. Immunology, vol. 224(4), PP: 511-517.
48. Lubin D M, Atkinson J P (1998). Decay-accelerating factor: Biochemistry, molecular-biology, and function. Ann. Rev. Immunol., vol. 7, PP: 35-58.
49. Williams P, Chaudry Y, Goodfellow I G, Billington J, Powell R, Spiller O B, Evans D J, Lea S (2003). Mapping CD55 function. J. Biol. Chem., vol. 12, PP: 10691-10696.
50. Dho S H, Lim J C, Kim L K (2018). Beyond the role of CD55 as a complement component. Innune Netw., Vol. 18(1), e11.
51. Skerka C, Chen Q, Fremeaux-Bacchi V, Roumenina L T (2013). Complement factor H related proteins (CFHRs). Mol. Immunol., vol. 56(3), PP: 170-180.
52. Cipriani V, Lores-Motta L, He F, Fathalla D, Tilakaratna V, McHarg S, Bayatti N, Acar I E, Hoyng C B, Fauser S, Moore A T, Yates J R W, de Jong E K, Morgan B P, den Hollander A I, Bishop P N, Clark S J (2020) Increased circulating levels of Factor H-related Protein 4 are strongly associated with age-related macular degeneration. Nat. Commun. Vol. 11(1), PP778.
53. Lako M, Kavangh D, (2021). Revisiting the role of factor H in age-related macular degeneration: Insights from complement-mediated renal disease and rare genetic variants. Surv. Ophthamol., vol. 66(2), PP: 378-401.
54. Callis, J., Fromm, M., and Walbot, V. (1987). Introns increase gene expression in cultured maize cells. Genes Dev. 1, 1183-1200.
55. Rose A B (2019). Introns as gene regulators: a brick on the accelerator. Front. Genet., vol. 9; 672.
56. Haut D D, Pintel D J (1998). Intron definition is required for excision of the minute virus of mice small intron and definition of the upstream exon. J. Virol., vol. 72(3), PP: 1834-1843.
57. Thomas J, Storm T A, Huang Z, Kay M A (2004). Rapid uncoating of vector genomes is the key to efficient liver transduction with pseudotyped adeno-associated virus vectors. J. Virol., vol. 78(6), PP: 3110-3122.
58. Fisher K J, Gao G P, Weiitzman M D, DeMatteo R, Burda. J F, Wilson J M (1996). Transduction with recombinant adeno-associated virus for gene therapy is limited by leading-strand synthesis. J. Virol., vol. 70(1), PP: 520-532.
59. Thompson J D, Higgins D G, Gibson T J (1994). CLUSTAL W: improving the sensitivity of progressive multiple sequence alignment through sequence weighting, position-specific gap penalties and weight matric choice. Nucleic Acids Res., 22(22), 4673-4680.
60. Thompson J D, Gibson T J, Plewniak F, Jeanmougin F, Higgins D G (1997). The CLUSTAL_X windows interface: flexible strategies for multiple sequence alignment aided by quality analysis tools. Nucleic Acids Res., 25(24), 4876-4882.
61. Do D V, Rhoades W, Nguyen Q D (2020). Pharmacokinetic study of intravitreal aflibercept in humans with neovascular age-related macular degeneration. Retina., 40(4):643-647.

1. A genetic construct comprising a promoter operably linked to a first coding sequence, which encodes an anti-VEGF protein, and a second coding sequence, which encodes an anti-fibrotic protein.

2. A genetic construct according to claim 1, wherein the promoter is the cytomegalovirus (CMV) promoter, a fusion of the CMV early enhancer element and the first intron of chicken beta-actin gene (CAG), the vitelliform macular dystrophy protein-2 (VMD2) promoter, the human phosphoglycerate kinase-1 (PGK-1) promoter, or the EF1α promoter, optionally wherein the promoter comprises a nucleotide sequence substantially as set out in SEQ ID No: 1, 2, 3, 4, 5, 6, 7, 8 or 9, or a fragment or variant thereof.

3. A genetic construct according to either claim 1 or claim 2, wherein the first coding sequence comprises a nucleotide sequence encoding an anti-VEGF protein capable of capturing all soluble forms of VEGF, including VEGF-A, VEGF-B, VEGF-C, VEGF-D and/or placenta growth factor (PIGF).

4. A genetic construct according to any preceding claim, wherein the first coding sequence comprises a nucleotide sequence encoding an anti-VEGF protein that specifically captures VEGF-A, optionally wherein the anti-VEGF protein is capable of capturing all isoforms of VEGF-A, including VEGF-121, VEGF-145, VEGF-165, VEGF-183, VEGF-189 and/or VEGF-206.

5. A genetic construct according to any preceding claim, wherein the anti-VEGF protein is an anti-VEGF antibody, or antigen-binding fragment thereof, optionally wherein the anti-VEGF protein is a single chain variable fragment (SCVF).

6. A genetic construct according to any preceding claim, wherein the first coding sequence comprises a nucleotide sequence substantially as set out in any one of SEQ ID No: 11, 13, 15, 17, 19 or 21, or a fragment or variant thereof, and/or wherein the first coding sequence encodes an amino acid sequence substantially as set out in SEQ ID No: 10, 12, 14, 16, 18 or 20, or a fragment or variant thereof.

7. A genetic construct according to any preceding claim, wherein the anti-fibrotic protein is an anti-complement protein.

8. A genetic construct according to claim 7, wherein the anti-complement protein is capable of neutralising or attenuating complement activation.

9. A genetic construct according to either claim 7 or 8, wherein the anti-complement protein is capable of targeting the alternative pathway (AP) of the complement system, preferably wherein the anti-complement protein does not target the classical pathway (CP) and/or the lectin pathway (LP) of the complement system.

10. A genetic construct according to any one of claims 7-9, wherein the anti-complement protein is an anti-C3b or anti-Bb antibody, or antigen-binding fragment thereof, optionally wherein the anti-complement protein is a single chain variable fragment (SCVF).

11. A genetic construct according to any preceding claim, wherein the second coding sequence comprises a nucleotide sequence substantially as set out in SEQ ID No: 23 or 25, or a fragment or variant thereof, and/or wherein the second coding sequence encodes an amino acid sequence substantially as set out in SEQ ID No: 22 or 24, or a fragment or variant thereof.

12. A genetic construct according to claim 7, wherein the anti-complement protein is CD55, preferably soluble CD55 (sCD55).

13. A genetic construct according to claim 12, wherein the second coding sequence comprises a nucleotide sequence substantially as set out in SEQ ID No: 27, or a fragment or variant thereof, and/or wherein the second coding sequence encodes an amino acid sequence substantially as set out in SEQ ID No: 26, or a fragment or variant thereof.

14. A genetic construct according to claim 7, wherein the anti-complement protein is complement factor H related protein-1 (CFHR1).

15. A genetic construct according to claim 14, wherein the second coding sequence comprises a nucleotide sequence substantially as set out in SEQ ID No: 29 or 30, or a fragment or variant thereof, and/or wherein the second coding sequence encodes an amino acid sequence substantially as set out in SEQ ID No: 28, or a fragment or variant thereof.

16. A genetic construct according to claim 7, wherein the anti-complement protein is CD46, preferably soluble CD46 (sCD46).

17. A genetic construct according to claim 16, wherein the second coding sequence comprises a nucleotide sequence substantially as set out in SEQ ID No: 32, or a fragment or variant thereof, and/or wherein the second coding sequence encodes an amino acid sequence substantially as set out in SEQ ID No: 31, or a fragment or variant thereof.

18. A genetic construct according to claim 7, wherein the anti-complement protein is Complement Factor H-Like protein 1 (CFHL1).

19. A genetic construct according to claim 18, wherein the second coding sequence comprises a nucleotide sequence substantially as set out in SEQ ID No: 98, or a fragment or variant thereof, and/or wherein the second coding sequence encodes an amino acid sequence substantially as set out in SEQ ID No: 97, or a fragment or variant thereof.

20. A genetic construct according to any one of claims 1-6, wherein the anti-fibrotic protein is capable of neutralising connective tissue growth factor (CTGF).

21. A genetic construct according to claim 20, wherein the anti-fibrotic protein is an anti-connective tissue growth factor (anti-CTGF) antibody, or antigen binding fragment thereof, preferably wherein the anti-CTGF antibody is an anti-CTGF single chain variable fragment (anti-CTGF SCVF).

22. A genetic construct according to claim 21, wherein the second coding sequence comprises a nucleotide sequence substantially as set out in SEQ ID No: 34 or 36, or a fragment or variant thereof, and/or wherein the second coding sequence encodes an amino acid sequence substantially as set out in SEQ ID No: 33 or 35, or a fragment or variant thereof.

23. A genetic construct according to any preceding claim, wherein the genetic construct comprises a spacer sequence disposed between the first and second coding sequences, which spacer sequence encodes a peptide spacer that is configured to produce the anti-VEGF protein and the anti-fibrotic protein as separate molecules.

24. A genetic construct according to claim 23, wherein the spacer sequence comprises and encodes a viral peptide spacer sequence, most preferably a viral-2A peptide spacer sequence.

25. A genetic construct according to claim 24, wherein the viral-2A peptide spacer sequence comprises a F2A, E2A, T2A or P2A sequence.

26. A genetic construct according to any one of claims 23-25, wherein the spacer sequence comprises a nucleotide sequence substantially as set out in SEQ ID No: 38, 40, 42 or 44, or a fragment or variant thereof, and/or wherein the spacer sequence encodes an amino acid sequence substantially as set out in SEQ ID No: 37, 39, 41 or 43, or a fragment or variant thereof.

27. A genetic construct according to any one of claims 23-25, wherein the genetic construct comprises a viral-2A removal sequence, optionally wherein the viral-2A removal sequence is disposed 5′ of the viral-2A sequence.

28. A genetic construct according to claim 27, wherein the viral-2A removal sequence is separated from the viral-2A peptide spacer sequence by a linker sequence comprising a tripeptide glycine-serine-glycine sequence (G-S-G).

29. A genetic construct according to either claim 27 or claim 28, wherein the viral-2A removal sequence is a furin recognition sequence, optionally wherein the viral-2A removal sequence encodes an amino acid sequence substantially as set out in SEQ ID No: 45, or a fragment or variant thereof.

30. A genetic construct according to claim 29, wherein the viral-2A removal sequence comprises a nucleotide sequence substantially as set out in SEQ ID No: 47, 49 or 51, or a fragment or variant thereof, and/or wherein the viral-2A removal sequence encodes an amino acid sequence substantially as set out in SEQ ID No: 46, 48 or 50, or a fragment or variant thereof.

31. A genetic construct according to either claim 27 or claim 28, wherein the viral-2A removal sequence is a gelatinase MMP-2 recognition sequence, optionally wherein the viral-2A removal sequence comprises a nucleotide sequence substantially as set out in SEQ ID No: 56, or a fragment or variant thereof, and/or wherein the viral-2A removal sequence encodes an amino acid sequence substantially as set out in SEQ ID No: 55, or a fragment or variant thereof.

32. A genetic construct according to either claim 27 or claim 28, wherein the viral-2A removal sequence is a renin recognition sequence, optionally wherein the viral-2A removal sequence comprises a nucleotide sequence substantially as set out in SEQ ID No: 60, or a fragment or variant thereof, and/or wherein the viral-2A removal sequence encodes an amino acid sequence substantially as set out in SEQ ID No: 59, or a fragment or variant thereof.

33. A genetic construct according to any preceding claim, wherein the genetic construct comprises a nucleotide sequence encoding Woodchuck Hepatitis Virus Post-transcriptional Regulatory Element (WPRE), optionally wherein the WPRE comprises a nucleic acid sequence substantially as set out in SEQ ID No: 61 or 62, or a fragment or variant thereof.

34. A genetic construct according to any preceding claim, wherein the genetic construct comprises a nucleotide sequence encoding a polyA tail, optionally wherein the polyA tail comprises a nucleic acid sequence substantially as set out in SEQ ID No: 63, 64 or 99, or a fragment or variant thereof.

35. A genetic construct according to any preceding claim, wherein the genetic construct comprises a nucleotide sequence encoding left and/or right Inverted Terminal Repeat sequences (ITRs), optionally wherein the left and/or right Inverted Terminal Repeats comprise a nucleic acid sequence substantially as set out in SEQ ID No: 65 or 66, or a fragment or variant thereof.

36. A genetic construct according to any preceding claim, wherein the genetic construct comprises a non-coding intron, optionally wherein the non-coding intron is located between the promoter and the first coding sequence.

37. A genetic construct according to claim 36, wherein the non-coding intron comprises a nucleic acid sequence substantially as set out in SEQ ID No: 67, 68 or 69, or a fragment or variant thereof.

38. A genetic construct according to any preceding claim, wherein the genetic construct comprises a signal peptide coding sequence, optionally wherein the signal peptide coding sequence comprises a nucleotide sequence substantially as set out in any one of SEQ ID No: 71, 73, 75, 77 or 70, or a fragment or variant thereof, and/or wherein the signal peptide coding sequence encodes an amino acid sequence substantially as set out in SEQ ID No: 70, 72, 74, 76 or 78, or a fragment or variant thereof.

39. A genetic construct according to any preceding claim, wherein the genetic construct encodes an amino acid sequence substantially as set out in SEQ ID No: 80, 82, 84, 86, 88, 90, 92, 94, 100, 102, 104 or 106, or a fragment or variant thereof, and/or wherein the construct comprises a nucleotide sequence substantially as set out in SEQ ID No: 81, 83, 85, 87, 89, 91, 93, 95, 101, 103, 105 or 107, or a fragment or variant thereof.

40. A recombinant vector comprising the genetic construct according to any one of claims 1-39.

41. A recombinant vector according to claim 40, wherein the vector is a recombinant AAV (rAAV) vector.

42. A recombinant vector according to claim 40 or claim 41, wherein the rAAV is AAV-1, AAV-2, AAV-3A, AAV-3B, AAV-4, AAV-5, AAV-6, AAV-7, AAV-8, AAV-9, AAV-10, AAV-11 or AAV-2.7m8.

43. A recombinant vector according to any either claim 41 or claim 42, wherein the rAAV is rAAV serotype-2.

44. A recombinant vector according to any one of claims 40-43, wherein the recombinant vector comprises a nucleotide sequence substantially as set out in SEQ ID No: 96, or a fragment or variant thereof.

45. The genetic construct according to any one of claims 1-39, or the recombinant vector according to any one of claims 40-44, for use as a medicament or in therapy.

46. The genetic construct according to any one of claims 1-39, or the recombinant vector according to any one of claims 40-44, for use in treating, preventing or ameliorating a retinal disorder, or for reducing vascular leakage and retinal cell damage.

47. The genetic construct or vector, for use according to claim 46, wherein the retinal disorder that is treated is: wet age-related macular degeneration; a diabetic retinopathy; any retinal disorder associated with diabetes; diabetic macular oedema (DMO); or a pathophysiological condition which involves vascular leakage and a resultant damage to retinal structures.

48. The genetic construct or vector, for use according to claim 47, wherein the retinal disorder is wet age-related macular degeneration.

49. The genetic construct or vector, for use according to claim 46, wherein the construct or vector is used to reduce vascular leakage and retinal cell damage associated with any one of the following conditions: diabetic retinopathy, cancer, systemic capillary leak syndrome (SCLS)/Clarkson's syndrome, angioedema, severe trauma, shock, sepsis, multiple organ dysfunction syndrome (MODS), chronic kidney disease, end-stage renal disease, Kawasaki disease, severe Ebola virus disease, Dengue virus infection and/or mycobacterial infection.

50. A pharmaceutical composition comprising the genetic construct according to any one of claims 1-39, or the recombinant vector according to any one of claims 40-44, and a pharmaceutically acceptable vehicle.

51. A method of preparing the pharmaceutical composition according to claim 50, the method comprising contacting the genetic construct according to any one of claims 1-39, or the recombinant vector according to any one of claims 40-44, with a pharmaceutically acceptable vehicle.
